(19) United States
(12) Patent Application Publication
(10) Pub. No.: US 2023/0073214 A1
(43) Pub. Date: Mar. 9, 2023

(54) BIOACTIVE RELEASING MEMBRANE FOR ANALYTE SENSOR

(71) Applicant: DexCom, Inc., San Diego, CA (US)

(72) Inventors: Shanger Wang, San Diego, CA (US); Mahender Nath Avula, San Diego, CA (US); Chris Dring, San Diego, CA (US); Ted Tang Lee, San Diego, CA (US); Xiangyou Liu, San Diego, CA (US); Shane Richard Parnell, San Diego, CA (US); Jiong Zou, San Diego, CA (US)

(73) Assignee: DexCom, Inc., San Diego, CA (US)

(21) Appl. No.: 17/945,585

(22) Filed: Sep. 15, 2022

Related U.S. Application Data
(63) Continuation-in-part of application No. 17/697,701, filed on Mar. 17, 2022.
(60) Provisional application No. 63/163,651, filed on Mar. 19, 2021.
(60) Provisional application No. 63/244,644, filed on Sep. 15, 2021.
(60) Provisional application No. 63/318,901, filed on Mar. 11, 2022.

(51) Int. Cl. A61K 9/00; A61B 5/145; A61B 5/1486; A61K 31/573

(52) U.S. Cl. CPC A61K 9/0024; A61B 5/14532; A61B 5/14865; A61K 31/573; A61B 2560/06

(57) ABSTRACT

The present disclosure relates generally to bioactive releasing membranes utilized with implantable devices, such as devices for the detection of analyte concentrations in a biological sample. More particularly, the disclosure relates to novel bioactive releasing membranes, to devices and implantable devices including these membranes, methods for forming the bioactive releasing membranes on or around the implantable devices, and to methods for monitoring analyte levels in a biological fluid sample using an implantable analyte detection device.

### CROSS-REFERENCE TO RELATED APPLICATIONS

This application is a continuation-in-part of and claims priority to U.S. application Ser. No. 17/697,701, filed Mar. 17, 2022, which claims priority to and the benefit of U.S. Provisional Application No. 63/163,651 filed on Mar. 19, 2021, and U.S. Provisional Application No. 63/244,644 filed on Sep. 15, 2021, and this application also claims priority to and the benefit of U.S. Provisional Application No. 63/318,901 filed on Mar. 11, 2022, all of which are hereby incorporated by reference in their entirety.

### FIELD

The present disclosure relates generally to bioactive releasing or eluting layers or membranes utilized with implantable devices, such as devices for the detection of analyte concentrations in a biological sample. More particularly, the disclosure relates to novel bioactive releasing membranes, to devices and implantable devices including these membranes, methods for forming the bioactive releasing membranes on or around the implantable devices, methods of improving and/or extending sensor life, and to methods for monitoring one or more analyte levels in a biological fluid sample using an implantable analyte detection device.

### BACKGROUND

One of the most heavily investigated analyte sensing devices is the implantable glucose device for detecting glucose levels in hosts with diabetes. Despite the increasing number of individuals diagnosed with diabetes and recent advances in the field of implantable glucose monitoring devices, currently used devices are unable to provide data safely and reliably for certain periods of time due to local tissue responses. By way of example, are two commonly used types of subcutaneously implantable glucose sensing devices. These types include those that are implanted transcutaneously and those that are wholly implanted.

### SUMMARY

In one aspect, a device for measurement of a concentration an analyte is provided, the device comprising: a sensor substrate comprising a distal end separated from a proximal end, and at least one sensor portion positioned between the distal end and the proximal end, the sensor portion configured to generate a signal associated with the concentration of the analyte; and a bioactive releasing membrane adjacent the sensor substrate, the bioactive releasing membrane comprising at least one releasable bioactive agent capable of modifying a tissue response of a subject.

In one aspect, the distal end has an outer surface and the bioactive releasing membrane is positioned on the outer surface.

In one aspect, alone or in combination with any one of the previous aspects, the bioactive releasing membrane is positioned only at the distal end.

In one aspect, alone or in combination with any one of the previous aspects, the bioactive releasing membrane is directly adjacent a resistance membrane.

In one aspect, alone or in combination with any one of the previous aspects, the bioactive releasing membrane is directly adjacent an interference membrane.

In one aspect, alone or in combination with any one of the previous aspects, the bioactive releasing membrane is directly adjacent an electrode membrane.

In one aspect, alone or in combination with any one of the previous aspects, the device further comprises a dissolvable coating adjacent the bioactive releasing membrane.

In one aspect, alone or in combination with any one of the previous aspects, the dissolvable coating further comprises a releasable bioactive agent.

In one aspect, alone or in combination with any one of the previous aspects, the at least one releasable bioactive agent is a first releasable bioactive agent, and the dissolvable coating further comprises a second releasable bioactive agent, the first releasable bioactive agent is the same or different from the second releasable bioactive agent.

In one aspect, alone or in combination with any one of the previous aspects, the dissolvable coating comprises the second releasable bioactive agent in combination with nanoparticles comprising one or more anti-inflammatory agents.

In one aspect, alone or in combination with any one of the previous aspects, the dissolvable coating provides bolus release of both the second releasable bioactive agent and the nanoparticles.

In one aspect, alone or in combination with any one of the previous aspects, the dissolvable coating is hydrophilic.

In one aspect, alone or in combination with any one of the previous aspects, the dissolvable coating is analyte diffusionable.

In one aspect, alone or in combination with any one of the previous aspects, the device further comprises a diffusion adjustment membrane adjacent the bioactive releasing membrane, wherein diffusion adjustment membrane is different from the bioactive releasing membrane.

In one aspect, alone or in combination with any one of the previous aspects, the diffusion adjustment membrane is directly adjacent the bioactive releasing membrane.

In one aspect, alone or in combination with any one of the previous aspects, the diffusion adjustment membrane is a block copolymer.

In one aspect, alone or in combination with any one of the previous aspects, the diffusion adjustment membrane is a segmented block copolymer.

In one aspect, alone or in combination with any one of the previous aspects, the diffusion adjustment membrane is a multi-block copolymer.

In one aspect, alone or in combination with any one of the previous aspects, the diffusion adjustment membrane is annealed.

In one aspect, alone or in combination with any one of the previous aspects, the annealed diffusion adjustment membrane comprises stable separated phases.

In one aspect, alone or in combination with anyone of the previous aspects, the stable separated phases provide diffusion channels for the at least one releasable bioactive agent.

In one aspect, alone or in combination with any one of the previous aspects, the bioactive releasing membrane comprises a soft segment and a hard segment comprising urethane groups, urea groups, or a combination of urethane groups and urea groups.

In one aspect, alone or in combination with any one of the previous aspects, the soft segment is two or more different polymer segments.

In one aspect, alone or in combination with any one of the previous aspects, the soft segment comprises a hydrophobic block and a hydrophilic block.

In one aspect, alone or in combination with any one of the previous aspects, the bioactive releasing membrane comprises a multicomponent soft segment comprising two or more different polymer segments.

In one aspect, alone or in combination with any one of the previous aspects, the multicomponent soft segment comprises a hydrophobic block and a hydrophilic block of a combination of at least one of a polysiloxane, a polyalkylcarbonate, and a polycarbonate with a polyalkylether, a polyalkylester.

In one aspect, alone or in combination with any one of the previous aspects, the soft segment comprises a combination of one or more of polysiloxane, polyalkylether, polyalkylester, polyalkylcarbonate, polycarbonate, and polysiloxane-polyalkylether segmented blocks and wherein the hard segement comprises at least one of norbornane diisocyanate (NBDI), isophorone diisocynate (IPDI), tolylene diisocynate (TDI), 1,3-phenylene diisocyanate (MPDI), trans-1,3-bis(isocynatomethyl) cyclohexane (1,3-H6XDI), bicyclohexylmethane-4,4′-diisocynate(HMDI), 4,4′-Diphenylmethane diisocynate (MDI), trans-1,4-bis(isocynatomethyl) cyclohexane (1,4-H6XDI), 1,4-cyclohexyl diisocynate (CHDI), 1,4-phenylene diisocynate (PPDI), 3,3′-Dimethyl-4,4′-biphenyldiisocyanate (TODI), and 1,6-hexamethylene diisocyanate (HDI).

In one aspect, alone or in combination with any one of the previous aspects, the soft segment comprises polysiloxane, polyalkylether, polyalkylester, polyalkylcarbonate, polycarbonate, or polysiloxane-polyalkylether segmented blocks.

In one aspect, alone or in combination with any one of the previous aspects, the bioactive releasing membrane further comprises a chain extender.

In one aspect, alone or in combination with any one of the previous aspects, the bioactive releasing membrane is a polyurethane urea.

In one aspect, alone or in combination with any one of the previous aspects, the bioactive releasing membrane comprises about 10-30 wt. % polysiloxane and about 10-30 wt. % polyalkylether, 40-60% wt. % hard segment comprising urethane groups, urea groups, or a combination of urethane groups and urea groups, and any remainder wt. % being chain extender, based on a total weight of the bioactive releasing membrane.

In one aspect, alone or in combination with any one of the previous aspects, the bioactive releasing membrane comprises about 20-30 wt. % polysiloxane, about 20-30 wt. % polyalkylether, and about 40-60 wt. % hard segment, and any remainder wt. % being chain extender, based on a total weight of the bioactive releasing membrane.

In one aspect, alone or in combination with any one of the previous aspects, the bioactive releasing membrane comprises a soft segment comprising the about 10-30 wt. % polysiloxane, the about 10-30 wt. % polyalkylether, and the about 0-10 wt. % chain extender, based on a total weight of the bioactive releasing membrane.

In one aspect, alone or in combination with any one of the previous aspects, the polyalkylether is represented by repeating units of formula (I): —(R5-O)—; where R5 is a linear or branched alkyl group of 2 to 6 carbons.

In one aspect, alone or in combination with any one of the previous aspects, the bioactive releasing membrane has a water uptake at equilibrium of between 1 wt. % to 4 wt. %.

In one aspect, alone or in combination with any one of the previous aspects, the bioactive releasing membrane has less than 3 wt. % water uptake at equilibrium.

In one aspect, alone or in combination with any one of the previous aspects, the bioactive releasing membrane is an excipient of the at least one releasable bioactive agent.

In one aspect, alone or in combination with any one of the previous aspects, the bioactive releasing membrane comprises a hydrophobic soft segment, at least one hydrophilic soft segment, and a hard segment comprising urethane groups, urea groups, or a combination of urethane groups and urea groups.

In one aspect, alone or in combination with any one of the previous aspects, the bioactive releasing membrane comprises hard segment and a soft segment, with the hard segment having an Hilderbrand solubility parameter closer to the at least one releasable bioactive agent than the soft segment.

In one aspect, alone or in combination with any one of the previous aspects, the distal end of the substrate comprises a wire singulation, a planar singulation, or a substantially planar singulation.

In one aspect, alone or in combination with any one of the previous aspects, the device further comprises an electrically insulating end-cap adjacent the distal end.

In one aspect, alone or in combination with any one of the previous aspects, the electrically insulating end-cap is a hydrophobic coating.

In one aspect, alone or in combination with any one of the previous aspects, the electrically insulating end-cap is non-permeable to electrochemically active species.

In one aspect, alone or in combination with any one of the previous aspects, the electrically insulating end-cap is non-permeable to the analyte.

In one aspect, alone or in combination with any one of the previous aspects, the electrically insulating end-cap extends longitudinally from the distal end.

In one aspect, alone or in combination with any one of the previous aspects, the electrically insulating end-cap extends from the distal end up to the sensor portion.

In one aspect, alone or in combination with any one of the previous aspects, the electrically insulating end-cap is a thermoplastic silicone polycarbonate polyurethane, polyacrylate, urethane acrylate, polybutadiene modified urethane, polyethylene vinyl acetate, silicone, or combinations thereof.

In another example, a method of reducing or delaying an immune response in a tissue of a subject is provided, the method comprising: (i) providing a continuous analyte sensing device, the device comprising: an insertable portion operably coupled to a non-insertable portion, the insertable portion comprising a sensing portion configured to be inserted into the tissue, the insertable portion having an insertable surface area and an insertable volume; at least one bioactive releasing membrane disposed over a portion of the insertable surface area, the bioactive releasing membrane being spatially separated from the sensing portion, the at least one bioactive releasing membrane comprising at least one bioactive agent; (ii) forming a tissue insertion volume in the tissue by inserting the insertable portion, the tissue insertion volume being greater than or equal to the insertable volume; (iii) releasing the at least one bioactive agent from the at least one bioactive releasing membrane into the tissue insertion volume at an average release rate from about 0.1 μg/day to about 5 μg/day; and (iv) reducing or delaying the immune response in the tissue.

In one aspect, the bioactive releasing membrane is spatially separated from the sensing portion.

In one aspect, alone or in combination with any one of the previous aspects, the bioactive releasing membrane active further comprises a non-releasable bioactive agent.

In one aspect, alone or in combination with any one of the previous aspects, the bioactive releasing membrane comprises a polymer and a weight/weight ratio of the at least one bioactive agent to the polymer is from about 0.1 to about 2, including all ranges and subranges therebetween.

In one aspect, alone or in combination with any one of the previous aspects, the at least one bioactive agent comprises an anti-inflammatory compound or a tissue response modifier.

In one aspect, alone or in combination with any one of the previous aspects, the at least one bioactive agent comprises dexamethasone, a dexamethasone salt, a dexamethasone derivative, dexamethasone acetate, or a combination of a dexamethasone salt, a dexamethasone derivative or dexamethasone acetate with dexamethasone.

In another example, a method of reducing signal noise in a continuous analyte sensor device caused by a foreign body response is provided, the method comprising: providing a continuous analyte sensing device comprising: a substrate comprising an insertable portion operably coupled to a non-insertable portion, the insertable portion having a distal end; at least one sensing portion positioned proximal from the distal end; at least one bioactive releasing membrane disposed over at least a portion of the distal end, the bioactive releasing membrane comprising at least one bioactive agent capable of attenuating a foreign body response; and reducing the signal noise during use of the continuous analyte sensing device.

In one aspect the method further comprises releasing or exposing the at least one bioactive agent to the tissue.

In one aspect, alone or in combination with any one of the previous aspects, the method further comprises attenuating the foreign body response in proximity to the distal end.

In one aspect, alone or in combination with any one of the previous aspects, the analyte is glucose and the signal noise is maintained at less than 4 mg/dL for at least 10 days.

In one aspect, alone or in combination with any one of the previous aspects, the analyte is glucose and the signal noise is maintained at less than 4 mg/dL for at least 15 days.

In one aspect, alone or in combination with any one of the previous aspects, the analyte is glucose and the signal noise is maintained at less than 4 mg/dL for at least 21 days.

In one aspect, alone or in combination with any one of the previous aspects, the insertable portion comprises an insertable surface area and an insertable volume.

In one aspect, alone or in combination with any one of the previous aspects, the at least one bioactive releasing membrane is disposed over a portion of the insertable surface area, the at least one bioactive releasing membrane having at least one of a bioactive releasing membrane surface area less than or equal to the insertable surface area.

In one aspect, alone or in combination with any one of the previous aspects, the at least one bioactive releasing membrane comprises a polymer and a weight ratio of the polymer to a total amount of at least one of the bioactive agent is between about 0.1 to about 2.

In one aspect, alone or in combination with any one of the previous aspects, the at least one bioactive agent comprises an anti-inflammatory compound or a tissue response modifier.

In one aspect, alone or in combination with any one of the previous aspects, the at least one bioactive agent comprises dexamethasone, a dexamethasone salt, a dexamethasone derivative, dexamethasone acetate, or a combination of a dexamethasone salt, a dexamethasone derivative or dexamethasone acetate with dexamethasone.

In one aspect, alone or in combination with any one of the previous aspects, the insertable portion and non-insertable portion are disposed on the substrate, the substrate is a wire, a In one aspect, alone or in combination with any one of the previous aspects, planar substrate or a substantially planar substrate, and the distal end further comprises a singulation.

In one aspect, alone or in combination with any one of the previous aspects, the method further comprises an electrically insulating end-cap adjacent the distal end.

In one aspect, alone or in combination with any one of the previous aspects, the electrically insulating end-cap is a hydrophobic coating.

In one aspect, alone or in combination with any one of the previous aspects, the electrically insulating end-cap extends longitudinally from the distal end.

In one aspect, alone or in combination with any one of the previous aspects, the electrically insulating end-cap is non-permeable to electrochemically active species.

In one aspect, alone or in combination with any one of the previous aspects, the electrically insulating end-cap is non-permeable to the analyte.

In one aspect, alone or in combination with any one of the previous aspects, the electrically insulating end-cap extends from the distal end up to the sensor portion.

In one aspect, alone or in combination with any one of the previous aspects, the electrically insulating end-cap is a thermoplastic silicone polycarbonate polyurethane, polyacrylate, urethane acrylate, polybutadiene modified urethane, polyethylene vinyl acetate, silicone, or combinations thereof.

In yet another example, a method of reducing an onset of loss in sensitivity of a continuous analyte sensor device caused by a foreign body response in tissue during use is provided, the method comprising: providing the continuous analyte sensing device comprising: a substrate comprising an insertable portion having a distal end operably coupled to a non-insertable portion; at least one sensing portion positioned proximal from the distal end and distal from the non-insertable portion; at least one bioactive releasing membrane disposed over a portion of the distal end, the at least one bioactive releasing membrane comprising at least one bioactive agent capable of attenuating a foreign body response; and reducing the onset of loss in sensitivity of the continuous analyte sensing device during use.

In one aspect, a method further comprises releasing or exposing the at least one bioactive agent to the tissue.

In one aspect, alone or in combination with any one of the previous aspects, the reducing the onset of the loss in sensitivity is for at least 14 days.

In one aspect, alone or in combination with any one of the previous aspects, the reducing the onset of the loss in sensitivity is for at least 20 days.

In one aspect, alone or in combination with any one of the previous aspects, the reducing the onset of the loss in sensitivity is for at least 30 days.

In one aspect, alone or in combination with any one of the previous aspects, the substrate is a wire, planar substrate, or substantially planar substrate and the distal end further comprises a singulation.

In one aspect, alone or in combination with any one of the previous aspects, the method further comprises an electrically insulating end-cap adjacent the distal end.

In one aspect, alone or in combination with any one of the previous aspects, the electrically insulating end-cap is different from the bioactive releasing membrane.

In one aspect, alone or in combination with any one of the previous aspects, the electrically insulating end-cap is a hydrophobic coating.

In one aspect, alone or in combination with any one of the previous aspects, the electrically insulating end-cap extends longitudinally from the distal end.

In one aspect, alone or in combination with any one of the previous aspects, the electrically insulating end-cap is non-permeable to electrochemically active species.

In one aspect, alone or in combination with any one of the previous aspects, the electrically insulating end-cap is non-permeable to the analyte.

In one aspect, alone or in combination with any one of the previous aspects, the electrically insulating end-cap extends longitudinally from the distal end up to the sensor portion.

In one aspect, alone or in combination with any one of the previous aspects, the electrically insulating end-cap is a thermoplastic silicone polycarbonate polyurethane, polyacrylate, urethane acrylate, polybutadiene modified urethane, polyethylene vinyl acetate, silicone, or combinations thereof.

In one aspect, alone or in combination with any one of the previous aspects, the at least one bioactive agent comprises an anti-inflammatory compound or tissue response modifier.

In one aspect, alone or in combination with any one of the previous aspects, the at least one bioactive agent comprises dexamethasone, a dexamethasone salt, a dexamethasone derivative, dexamethasone acetate, or a combination of a dexamethasone salt, a dexamethasone derivative or dexamethasone acetate with dexamethasone.

In yet another example, a device for measurement of a concentration of an analyte is provided, the device comprising: a sensor portion configured to generate a signal associated with the concentration of the analyte; and a bioactive releasing membrane in proximity to the sensor portion, the bioactive releasing membrane configured to form a complex with at least one bioactive agent, the at least one bioactive agent configured to be released from the bioactive releasing membrane to modify a tissue response of a subject.

In one aspect, the complex with the at least one bioactive agent is covalent or non-covalent.

In one aspect, alone or in combination with any one of the previous aspects, the complex with the at least one bioactive agent is ionic.

In one aspect, alone or in combination with any one of the previous aspects, the complex is with the at least one bioactive agent provides a bioactive agent conjugate.

In one aspect, alone or in combination with any one of the previous aspects, the at least one bioactive agent comprises an anti-inflammatory compound or a tissue response modifier.

In one aspect, alone or in combination with any one of the previous aspects, the at least one bioactive agent comprises dexamethasone, a dexamethasone salt, a dexamethasone derivative, dexamethasone acetate, or a combination of a dexamethasone salt, a dexamethasone derivative or dexamethasone acetate with dexamethasone.

In one aspect, alone or in combination with any one of the previous aspects, the at least one bioactive agent is a nitric oxide releasing molecule, polymer, or oligomer.

In one aspect, alone or in combination with any one of the previous aspects, the nitric oxide releasing molecule is selected from N-diazeniumdiolates and S-nitrosothiols.

In one aspect, alone or in combination with any one of the previous aspects, the at least one bioactive agent is covalently coupled Factor H.

In one aspect, alone or in combination with any one of the previous aspects, the complex is a bioactive agent conjugate comprising a borate ester or boronate.

In one aspect, alone or in combination with any one of the previous aspects, the complex is a bioactive agent conjugate comprises at least one cleavable linker that is cleavable by a subcutaneous stimuli.

In one aspect, alone or in combination with any one of the previous aspects, the subcutaneous stimuli is a matrix metallopeptidase or a protease attack.

In one aspect, alone or in combination with any one of the previous aspects, the bioactive releasing membrane comprises a hydrophilic hydrogel, the hydrophilic hydrogel is at least partly crosslinked and dissolvable in biological fluid.

In one aspect, alone or in combination with any one of the previous aspects, the hydrophilic hydrogel comprises hyaluronic acid crosslinked by divinyl sulfone or polyethylene glycol divinyl sulfone.

In one aspect, alone or in combination with any one of the previous aspects, the bioactive releasing membrane comprises silver nanoparticles.

In one aspect, alone or in combination with any one of the previous aspects, the bioactive releasing membrane comprises biodegradable polymeric nanoparticles selected from PLA, PLGA, PCL, PVL, PLLA, PDLA, PEO-b-PLA block copolymers, polyphosphoesters, or PEO-b-polypeptides comprising the at least one bioactive agent.

In one aspect, alone or in combination with any one of the previous aspects, the bioactive releasing membrane comprises an organic gel carrier and/or an inorganic gel carrier.

In one aspect, alone or in combination with any one of the previous aspects, the bioactive releasing membrane is configured to form the complex with the at least one bioactive agent comprises a combination of the least one bioactive agent encapsulated in the bioactive releasing membrane and the least one bioactive agent covalently coupled to the bioactive releasing membrane.

In one aspect, alone or in combination with any one of the previous aspects, the bioactive releasing membrane configured to form the complex with the at least one bioactive agent comprises spatially distal drug depots of the at least one bioactive agent.

In one aspect, alone or in combination with any one of the previous aspects, the bioactive releasing membrane configured to form the complex with the at least one bioactive agent comprises a hydrolytically degradable biopolymer comprising the at least one bioactive agent.

In one aspect, alone or in combination with any one of the previous aspects, the hydrolytically degradable biopolymer comprises a salicylic acid polyanhydride ester.

In one aspect, alone or in combination with any one of the previous aspects, the bioactive releasing membrane comprises polyurethane segments and/or polyurea segments, the polyurethane segments and/or the polyurea segments are from about 15 wt. % to about 75 wt. %, based on the total weight of the bioactive releasing membrane, including all ranges and subranges therebetween.

In one aspect, alone or in combination with any one of the previous aspects, the bioactive releasing membrane comprises at least one polymer segment, the at least one polymer segment selected from the group consisting of epoxides, polyolefins, polysiloxanes, polyamides, polystyrenes, polyacrylates, polyethers, polypyridines, polyesters, polyalkylesters, polyalkylcarbonate, polycarbonates, polyethylene vinyl acetate, polyvinyl alcohol, and copolymers thereof.

In one aspect, alone or in combination with any one of the previous aspects, the bioactive releasing membrane comprises a polyethylene oxide segment.

In one aspect, alone or in combination with any one of the previous aspects, the polyethylene oxide segment is from about 5 wt. % to about 60 wt. %, based on the total weight of the bioactive releasing membrane.

In one aspect, alone or in combination with any one of the previous aspects, a base polymer of the bioactive releasing membrane has an average molecular weight of from about 10 kDa to about 500 kDa, including all ranges and subranges therebetween.

In one aspect, alone or in combination with any one of the previous aspects, the bioactive releasing membrane has a polydispersity index of from 1 to about 10, including all ranges and subranges therebetween.

In one aspect, alone or in combination with any one of the previous aspects, the bioactive releasing membrane has a contact angle of from about 90° to about 160°, including all ranges and subranges therebetween.

In yet another example, a device for measurement of a concentration of an analyte is provided, the device comprising: a sensor portion configured to generate a signal associated with the concentration of the analyte; and a bioactive releasing membrane in proximity to the sensor portion, the bioactive releasing membrane comprising one or more zwitterionic repeating units complexed with at least one bioactive agent, the at least one bioactive agent configured to be released from the one or more zwitterionic repeating units to modify a tissue response of a subject.

In one aspect, the one or more zwitterionic repeating units comprise a betaine compound or derivative thereof.

In one aspect, alone or in combination with any one of the previous aspects, the one or more zwitterionic repeating units comprise a betaine compound or precursor thereof.

In one aspect, alone or in combination with any one of the previous aspects, the one or more zwitterionic repeating units comprise at least one moiety selected from the group consisting of a carboxyl betaine, a sulfo-betaine, a phosphor betaine, and derivatives thereof.

In one aspect, alone or in combination with any one of the previous aspects, the at least one bioactive agent comprises an anti-inflammatory compound or a tissue response modifier.

In one aspect, alone or in combination with any one of the previous aspects, the at least one bioactive agent comprises dexamethasone, a dexamethasone salt, a dexamethasone derivative, dexamethasone acetate, or a combination of a dexamethasone salt, a dexamethasone derivative or dexamethasone acetate with dexamethasone.

In one aspect, alone or in combination with any one of the previous aspects, the one or more zwitterionic repeating units are derived from a monomer selected from the group consisting of:

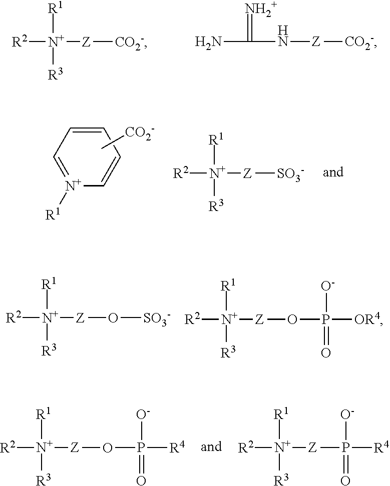

where Z is branched or straight chain alkyl, heteroalkyl, cycloalkyl, cycloheteroalkyl, aryl, or heteroaryl; R1 is H, alkyl, heteroalkyl, cycloalkyl, heterocycloalkyl, aryl, or heteroaryl; and R2, R3, and R4 are independently chosen from alkyl, heteroalkyl, cycloalkyl, heterocycloalkyl, aryl, or heteroaryl; and wherein one or more of R1, R2, R3, R4, and Z are substituted with a polymerization group.

In one aspect, alone or in combination with any one of the previous aspects, the polymerization group is selected from alkene, alkyne, epoxide, lactone, amine, hydroxyl, isocyanate, carboxylic acid, anhydride, silane, halide, aldehyde, carbodiimide or combinations thereof.

In one aspect, alone or in combination with any one of the previous aspects, the one or more zwitterionic repeating units is at least about 1 wt. % based on the total weight of the bioactive releasing membrane.

In one aspect, alone or in combination with any one of the previous aspects, the bioactive releasing membrane further comprises one or more zwitterions selected from the group consisting of cocamidopropyl betaine, oleamidopropyl betaine, octyl sulfobetaine, caprylyl sulfobetaine, lauryl sulfobetaine, myristyl sulfobetaine, palmityl sulfobetaine, stearyl sulfobetaine, betaine (trimethylglycine), octyl betaine, phosphatidylcholine, glycine betaine, poly(carboxybetaine), poly(sulfobetaine), and derivatives thereof.

In one aspect, alone or in combination with any one of the previous aspects, the bioactive releasing membrane comprises a polymer chain having zwitterionic groups at an end of the polymer chain and along the polymer chain.

In one aspect, alone or in combination with any one of the previous aspects, the bioactive releasing membrane comprises a polymer chain having both hydrophilic and hydrophobic regions and wherein one or more zwitterionic compounds are present at an end of the polymer chain; the bioactive releasing membrane comprising a base polymer selected from polyolefin, polystyrene, polyoxymethylene, polysiloxane, polyether, polyacrylic, polymethacrylic, polyester, polyalkylester, polyalkylcarbonate, polycarbonate, polyamide, polypyridine, poly(ether ketone), poly(ether imide), polyurethane, polyurethane urea, polyethylene vinyl acetate, polyvinyl alcohol, or copolymers or blends thereof.

In one aspect, alone or in combination with any one of the previous aspects, the base polymer of the bioactive releasing membrane has an average molecular weight of from about 10 kDa to about 500 kDa, including all ranges and subranges therebetween.

In one aspect, alone or in combination with any one of the previous aspects, the base polymer of the bioactive releasing membrane has a polydispersity index of from about 1 to about 10, including all ranges and subranges therebetween.

In one aspect, alone or in combination with any one of the previous aspects, the base polymer of the bioactive releasing membrane has a dynamic contact angle of from about 90° to about 160°, including all ranges and subranges therebetween.

### BRIEF DESCRIPTION OF THE DRAWINGS

FIG. 1A is a sectional view of an exemplary example of a continuous analyte sensing device.

FIG. 1B is a sectional view of an exemplary example of a continuous analyte sensing device.

FIG. 2A is a perspective view of an exemplary continuous analyte sensing device as disclosed and described herein.

FIG. 2B is a cross-sectional view through the continuous analyte sensing device of FIG. 2A along section line B-B of FIG. 2A.

FIG. 2C is a cross-sectional view through the continuous analyte sensing device of FIG. 2A along section line B-B of FIG. 2A showing an exemplary bioactive releasing layer.

FIG. 2D is a cross-sectional view through the continuous analyte sensing device of FIG. 2A on line D-D of FIG. 2A showing an exemplary bioactive releasing membrane as disclosed and described herein.

FIG. 2E is a cross-sectional view through the continuous analyte sensing device of FIG. 2A on line D-D of FIG. 2A showing another exemplary bioactive releasing membrane as disclosed and described herein.

FIG. 2F is a perspective view schematic illustrating an in vivo portion of an exemplary continuous analyte sensing device as disclosed and described herein.

FIG. 2G is a side-view schematic illustrating an in vivo portion of the exemplary sensor of FIG. 2F as disclosed and described herein.

FIG. 2H is a cross-sectional planar view of a continuous analyte sensing device in one example as disclosed and described herein.

FIG. 2I is a sectional view of a continuous analyte sensing device in one example as disclosed and described herein.

FIG. 2J is a cross-sectional view of a continuous analyte sensing device in one example as disclosed and described herein.

FIG. 3A is a side schematic view of a transcutaneous continuous analyte sensing device in one example as disclosed and described herein.

FIG. 3B is a side schematic view of a transcutaneous continuous analyte sensing device in an alternative example as disclosed and described herein.

FIG. 3C is a side schematic view of an implantable portion of an implantable continuous analyte sensing device in one example.

FIG. 3D is a side schematic view of an implantable portion of an implantable analyte sensor in an alternative example.

FIG. 3E is a side schematic view of an implantable portion of a continuous analyte sensing device in another alternative example.

FIG. 3F is a side view of one example of a continuous analyte sensing device inductively coupled to an electronics unit within a functionally useful distance on the host's skin.

FIG. 3G is a side view of one example of an implantable portion of a continuous analyte sensing device inductively coupled to an electronics unit implanted in the host's tissue at a functionally useful distance.

FIG. 3H is a side schematic view of an implantable portion of a continuous analyte sensing device in another alternative example.

FIG. 3I is a sectional view of an implantable portion of a continuous analyte sensing device in another alternative example.

FIG. 3J is a sectional view of an implantable portion of a continuous analyte sensing device in another alternative example.

FIG. 3K is a side schematic view of an implantable portion of a continuous analyte sensing device.

FIG. 3L is a side schematic view of an implantable portion of a continuous analyte sensing device in an alternative example.

FIG. 3M is a side schematic view of an implantable portion of a continuous analyte sensing device in another alternative example.

FIG. 3N is a side schematic view of an implantable portion of a continuous analyte sensing device in another alternative example.

FIG. 3O is a side schematic view of an implantable portion of a continuous analyte sensing device in another alternative example.

FIG. 3P is a graphical representation of in vivo bioactive agent release from a bioactive releasing membrane over time as disclosed and described herein.

FIG. 3Q is a graphical representation of in vivo bioactive agent release from a bioactive releasing membrane over time as disclosed and described herein.

FIG. 4A is a schematic view of a hard-soft segmented polymer as disclosed and described herein.

FIG. 4B a cross-sectional view through an exemplary membrane indicating a 3-D volume 4C.

FIG. 4C is a side schematic view of the 3-D volume 4C of FIG. 4B.

FIG. 5A is a graphical representation of cumulative release rate of a bioactive agent from a bioactive releasing membrane over time as disclosed and described herein.

FIG. 5B is a graphical representation of cumulative release rate of a bioactive agent from a bioactive releasing membrane over time as disclosed and described herein.

FIG. 5C is a graphical representation of cumulative release rate of a bioactive agent from different bioactive releasing membranes over time as disclosed and described herein.

FIG. 6A is a graphical representation of bioactive agent release from different bioactive releasing membrane with respect to their water uptake as disclosed and described herein.

FIG. 6B is a graphical representation of normalized sensitivity of sensors with and without bioactive releasing membranes over 18 days as disclosed and described herein.

FIG. 6C is a graphical representation of normalized sensitivity of sensors with and without bioactive releasing membranes over 30 days as disclosed and described herein.

FIG. 6D is a survival plot representation of normalized sensitivity of sensors with and without bioactive releasing membranes as disclosed and described herein.

FIG. 6E is a survival plot representation of normalized sensitivity of sensors with different bioactive releasing membranes as disclosed and described herein.

FIG. 7A is a graphical representation of mean absolute noise from a sensor with a bioactive releasing membrane over time as disclosed and described herein.

FIG. 7B is a survival plot representation of mean absolute noise of sensors with and without bioactive releasing membranes as disclosed and described herein.

FIG. 7C is a survival plot representation of mean absolute noise of sensors with different bioactive releasing membranes as disclosed and described herein.

FIG. 8A is histography image of foreign body response from a sensor without a bioactive releasing membrane.

FIG. 8B is histography image of foreign body response from a sensor with a bioactive releasing membrane as disclosed and described herein.

### DETAILED DESCRIPTION

The following description and examples illustrate a preferred example of the present disclosure in detail. Those of skill in the art will recognize that there are numerous variations and modifications of this disclosure that are encompassed by its scope. Accordingly, the description of an example should not be deemed to limit the scope of the present disclosure.

### Definitions

In order to facilitate an understanding of the disclosed examples, a number of terms are defined below.

The term “about” as used herein is a broad term, and is to be given its ordinary and customary meaning to a person of ordinary skill in the art (and is not be limited to a special or customized meaning), and refers without limitation to allowing for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The phrase “substantially free of” as used herein can mean having none or having a trivial amount of, such that the amount of material present does not affect the material properties of the composition including the material, such that about 0 wt. % to about 5 wt. % of the composition is the material, or about 0 wt. % to about 1 wt. %, or about 5 wt. % or less, or less than or equal to about 4.5 wt. %, 4, 3.5, 3, 2.5, 2, 1.5, 1, 0.9, 0.8, 0.7, 0.6, 0.5, 0.4, 0.3, 0.2, 0.1, 0.01, or about 0.001 wt. % or less, or about 0 wt. %.

The term “adhere” and “attach” as used herein are broad terms, and are to be given their ordinary and customary meaning to a person of ordinary skill in the art (and are not be limited to a special or customized meaning), and refer without limitation to hold, bind, or stick, for example, by gluing, bonding, grasping, interpenetrating, or fusing.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, metabolites, and/or reaction products. In some examples, the analyte measured by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bilirubin, biotinidase; biopterin; c-reactive protein; carnitine; carnosinase; CD4; ceruloplasmin; chenodeoxycholic acid; chloroquine; cholesterol; cholinesterase; conjugated 1-βhydroxy-cholic acid; cortisol; creatine; creatine kinase; creatine kinase MM isoenzyme; creatinine; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycerol; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; beta-hydroxybutyrate; ketones; lactate; lead; lipoproteins ((a), B/A-1, P); lysozyme; mefloquine; netilmicin; oxygen; phenobarbitone; phenytoin; phytanic/pristanic acid; potassium, sodium, and/or other blood electrolytes; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uric acid; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain examples. The analyte can be naturally present in the biological fluid, or endogenous, for example, a metabolic product, a hormone, an antigen, an antibody, and the like. Alternately, the analyte can be introduced into the body, or exogenous, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, RITALIN®, CYLERT®, PRELUDIN®, DIDREX®, PRESTATE®, VORANIL®, SANDREX®, PLEGINE®); depressants (barbiturates, methaqualone, tranquilizers such as VALIUM®, LIBRIUM®, MILTOWN®, SERAX®, EQUANIL®, TRANXENE®); hallucinogens (phencyclidine, lysergic acid, mescaline, peyote, psilocybin); narcotics (heroin, codeine, morphine, opium, meperidine, PERCOCET®, PERCODAN®, TUSSIONEX®, fentanyl, DARVON®, TALWIN, LOMOTIL®); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and histamine.

The phrases “analyte-measuring device,” “analyte-monitoring device,” “analyte-sensing device,” “continuous analyte sensing device,” “continuous analyte sensor device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing and/or detecting element.

The phrase “barrier cell layer” as used herein is a broad phrase, and is to be given its ordinary and customary meaning to a person of ordinary skill in the art (and is not to be limited to a special or customized meaning), and refers without limitation to a part of a foreign body response that forms a cohesive monolayer of cells (for example, macrophages and foreign body giant cells) that substantially block the transport of molecules and other substances to the implantable device.

The phrase and term “bioactive agent” and “bioactive” as used herein is a broad term, and is to be given its ordinary and customary meaning to a person of ordinary skill in the art (and is not to be limited to a special or customized meaning), and refers without limitation to any substance that has an effect on or elicits a response from living tissue, for example, drugs, biologics, reactive oxygen scavenger (ROS), and metal ions.

The phrases “biointerface membrane,” “biointerface domain,” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, continuous analyte sensing device, continuous analyte sensor device, and/or multi-analyte sensor device responsible for the detection of, or transduction of a signal associated with, a particular analyte or combination of analytes. In examples, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In examples, such biosensors and/or sensors are capable of providing specific quantitative, semi-quantitative, qualitative, semi qualitative analytical signals using a biological recognition element combined with a detecting and/or transducing element.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The phrase “cell processes” as used herein is a broad phrase, and is to be given its ordinary and customary meaning to a person of ordinary skill in the art (and is not to be limited to a special or customized meaning), and refers without limitation to pseudopodia of a cell.

The phrase “cellular attachment” as used herein is a broad phrase, and is to be given its ordinary and customary meaning to a person of ordinary skill in the art (and is not to be limited to a special or customized meaning), and refers without limitation to adhesion of cells and/or cell processes to a material at the molecular level, and/or attachment of cells and/or cell processes to microporous material surfaces or macroporous material surfaces. One example of a material used in the prior art that encourages cellular attachment to its porous surfaces is the BIOPORE™ cell culture support marketed by Millipore (Bedford, Mass.), and as described in Brauker et al., U.S. Pat. No. 5,741,330.

The term “comprising” as used herein is synonymous with “including,” “containing,” or “characterized by,” and is inclusive or open-ended and does not exclude additional, unrecited elements or method steps.

The term “conjugate” as used herein, is a broad term, and is to be given its ordinary and customary meaning to a person of ordinary skill in the art (and is not to be limited to a special or customized meaning), and refers without limitation to bioactive agents covalently linked through a linker to a carrier or nanocarrier, such as a polymer (e.g., the bioactive releasing membrane or biointerface layer), the linker being biologically active, as in capable of allowing the separation of the drug from the carrier when exposed or presented to a biological environment, such as a subcutaneous or transcutaneous environment. Conjugate, as used herein, is inclusive of drug releasing layer-bioactive agent conjugates and nanoparticle polymer-bioactive agent conjugates. Suitable carriers/nanocarriers include PEG and N-(2-hydroxypropyl) methacrylamide (HPMA), polyglutamic acid (PGA) and copolymers thereof. Conjugate, as used herein, is inclusive of drug releasing layer-bioactive agent conjugates and nanoparticle polymer-bioactive agent conjugates present in the drug releasing layer. In examples, the bioactive releasing membrane comprises domains having drug releasing-bioactive agent conjugates and domains having bioactive agent depots, where said domains can be spatially arranged vertically or horizontally.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrase “continuous analyte sensing” as used herein is a broad phrase, and is to be given its ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every 5 seconds or less to about 10 minutes or more. In further examples, monitoring of analyte concentration is performed from about every 10, 15, 20, 25, 30, 35, 40, 45, 50, 55, or 60 second to about 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another (i.e. “directly coupled”). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components.

The phrase “defined edges” as used herein is a broad phrase, and is to be given its ordinary and customary meaning to a person of ordinary skill in the art (and is not to be limited to a special or customized meaning), and refers without limitation to abrupt, distinct edges or borders among layers, domains, coatings, or portions. “Defined edges” are in contrast to a gradual transition between layers, domains, coatings, or portions.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of the membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations, for example, such as a host postprandial glucose concentrations. While not wishing to be bound by theory, it is believed that drift may be the result of a local decrease in glucose transport to the sensor, for example, due to a formation of a foreign body capsule (FBC). It is also believed that an insufficient amount of interstitial fluid surrounding the sensor may result in reduced oxygen and/or glucose transport to the sensor. In one example, an increase in local interstitial fluid may slow or reduce drift and thus improve sensor performance. Drift may also be the result of sensor electronics, or algorithmic models used to compensate for noise or other anomalies that can occur with electrical signals in ranges including the, microampere range, picoampere range, nanoampere range, and femtoampere range.

The phrases “bioactive releasing membrane” and “drug releasing layer” and “bioactive releasing domain” and “bioactive agent releasing membrane” are used interchangeably herein and are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In examples, the “bioactive releasing membrane” and “drug releasing layer” and “bioactive releasing domain” and “bioactive agent releasing membrane” can be comprised of two or more domains and is typically of a few microns thickness or more. In examples the bioactive releasing membrane and/or bioactive releasing membrane and/or bioactive agent releasing membrane and/or and bioactive agent releasing membrane are substantially the same as the biointerface layer and/or biointerface membrane. In another example, the bioactive releasing membrane and/or bioactive releasing membrane and/or bioactive agent releasing membrane and/or and bioactive agent releasing membrane are distinct from the biointerface layer and/or biointerface membrane.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In examples, hydrogen peroxide produced by an enzyme-catalyzed reaction of an analyte being detected reacts can create a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode. In another example, electron transfer is provided using a mediator or “wired enzyme” during reduction-oxidation (redox) of the transducing element and the analyte.

The phrase “hard segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts resistance properties, e.g., resistance to bending or twisting. The term “hard segment” can be further characterized as a crystalline, semi-crystalline, or glassy material with a glass transition temperature determined by dynamic scanning calorimetry (“Tg”) typically above ambient temperature, and is typically made of diisocyanate with or without chain extender.

The term “host” as used herein is a broad term, and is to be given its ordinary and customary meaning to a person of ordinary skill in the art (and is not to be limited to a special or customized meaning), and refers without limitation to mammals, for example humans.

The terms “implanted” or “implantable” as used herein are broad terms, and are to be given their ordinary and customary meaning to a person of ordinary skill in the art (and are not to be limited to a special or customized meaning), and refer without limitation to objects (e.g., sensors) that are inserted subcutaneously (i.e. in the layer of fat between the skin and the muscle) or transcutaneously (i.e. penetrating, entering, or passing through intact skin), which may result in a sensor that has an in vivo portion and an ex vivo portion.

The phrase “insertable surface area” as used herein is a broad phrase, and is to be given its ordinary and customary meaning to a person of ordinary skill in the art (and is not to be limited to a special or customized meaning), and refers without limitation to a surface area of an insertable portion of an analyte sensor including, but not limited to, the surface area of flat (substantially planar) and/or wire substrates utilized in the analyte sensor as described herein.

The phrase “insertable volume” as used herein is a broad phrase, and is to be given its ordinary and customary meaning to a person of ordinary skill in the art (and is not to be limited to a special or customized meaning), and refers without limitation to a volume ahead of and alongside a path of insertion of an insertable portion of an analyte sensor, as described herein, as well as an incision made in the skin to insert the insertable portion of the analyte sensor. The insertable volume also includes up to 5 mm radially or perpendicular to the volume ahead of and alongside the path of insertion.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In examples of an electrochemical sensor, interfering species are compounds with an oxidation potential that overlaps with the analyte to be measured or one or more mediators.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In examples, the membrane system comprises an immobilized glucose oxidase enzyme, which enables a reaction to occur between glucose and oxygen whereby a concentration of glucose can be measured.

The term “micro,” as used herein is a broad term, and is to be given its ordinary and customary meaning to a person of ordinary skill in the art (and is not to be limited to a special or customized meaning), and refers without limitation to a small object or scale of approximately 10-6 m that is not visible without magnification. The term “micro” is in contrast to the term “macro,” which refers to a large object that may be visible without magnification. Similarly, the term “nano” refers to a small object or scale of approximately 10-9 m.

The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a bioactive releasing membrane with at least one bioactive agent. For example, noise of one or more exemplary biosensors as presently disclosed can be determined and then compared qualitatively or quantitatively. By way of example, by obtaining a raw signal timeseries with a fixed sampling interval (in units of picoampere (pA)), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units of mg/dL, (the unit of “noise”), using a glucose sensitivity timeseries, in units of pA/mg/dL, where the glucose sensitivity timeseries is derived by using a mathematical model between the raw signal and reference blood glucose measurements (e.g., obtained from Blood Glucose Meter). Optionally, the timeseries can be aggregated as desired, e.g., by hour or day. Comparison of corresponding timeseries between different exemplary biosensors with the presently disclosed bioactive releasing membrane and one or more bioactive agents provides for qualitative or quantitative determination of improvement of noise.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The term “polyampholyte polymer” as used herein is a broad term, and is to be given its ordinary and customary meaning to a person of ordinary skill in the art (and is not to be limited to a special or customized meaning), and refers without limitation to polymers comprising both cationic and anionic groups. Such polymers can be prepared to have about equal numbers of positive and negative charges, and thus the surface of such polymers can be about net neutrally charged. Alternately, such polymers can be prepared to have an excess of either positive or negative charges, and thus the surface of such polymers can be net positively or negatively charged, respectively. “Polyampholyte polymer” is inclusive of polyampholytic polymers.

The phrase “polymerization group” used herein is a broad phrase, and is to be given its ordinary and customary meaning to a person of ordinary skill in the art (and is not to be limited to a special or customized meaning), and refers without limitation to a functional group that permits polymerization of the monomer with itself to form a homopolymer or together with different monomers to form a copolymer. Depending on the type of polymerization methods employed, the polymerization group can be selected from alkene, alkyne, epoxide, lactone, amine, hydroxyl, isocyanate, carboxylic acid, anhydride, silane, halide, aldehyde, and carbodiimide.

The term “polyzwitterions” as used herein is a broad term, and is to be given its ordinary and customary meaning to a person of ordinary skill in the art (and is not to be limited to a special or customized meaning), and refers without limitation to polymers where a repeating unit of the polymer chain is a zwitterionic moiety. Polyzwitterions are also known as polybetaines. Since polyzwitterions have both cationic and anionic groups, they are a type of polyampholytic polymer. They are unique, however, because the cationic and anionic groups are both part of the same repeating unit, which means a polyzwitterion has the same number of cationic groups and anionic groups whereas other polyampholytic polymers can have more of one ionic group than the other. Also, polyzwitterions have the cationic group and anionic group as part of a repeating unit. Polyampholytic polymers need not have cationic groups connected to anionic groups; they can be on different repeating units and thus may be distributed apart from one another at random intervals, or one ionic group may outnumber the other.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme layer. If the sensor is deemed to be the point of reference and the enzyme layer is positioned nearer to the sensor than the biointerface layer, then the enzyme layer is more proximal to the sensor than the biointerface layer.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and noncontinuous portions, coatings, domains, or layers. For example, a coating disposed around a sensing region but not about the sensing region is “semi-continuous.”

The phrases “sensing portion,” “sensing membrane,” “sensing region,” “sensing domain,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In examples, the sensing portion, sensing membrane, and/or sensing mechanism generally comprise an electrode configured to provide signals during electrochemically reactions with one or more membranes covering electrochemically reactive surface. In examples, such sensing portions, sensing membranes, and/or sensing mechanisms are capable of providing specific quantitative, semi-quantitative, qualitative, semi qualitative analytical signals using a biological recognition element combined with a detecting and/or transducing element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, or a protein, for example, one or more periplasmic binding protein (PBP) or mutant or fusion protein thereof having one or more analyte binding regions, each region capable of specifically and reversibly binding to at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, in the biological sample.

In examples, the sensing region or sensing portion can comprise at least a portion of a conductive substrate or at least a portion of a conductive surface, for example, a wire or conductive trace or a substantially planar substrate including substantially planar trace(s), and a membrane. In examples, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme layer, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) or mutant or fusion protein thereof having one or more analyte binding regions, each region capable of specifically and reversibly binding to at least one analyte. Mutations of the PBP can contribute to or alter one or more of the binding constants, extended stability of the protein, including thermal stability, to bind the protein to a special encapsulation matrix, membrane or polymer, or to attach a detectable reporter group or “label” to indicate a change in the binding region. Specific examples of changes in the binding region include, but are not limited to, hydrophobic/hydrophilic environmental changes, three-dimensional conformational changes, changes in the orientation of amino acid side chains in the binding region of proteins, and redox states of the binding region. Such changes to the binding region provide for transduction of a detectable signal corresponding to the one or more analytes present in the biological fluid.

In examples, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The sensing region transduces the recognition of analytes into a semi-quantitative or quantitative signal. Thus, “transducing” or “transduction” and their grammatical equivalents as are used herein encompasses optical, electrochemical, acoustical/mechanical, or colorimetrical technologies and methods. Electrochemical properties include current and/or voltage, capacitance, and potential. Optical properties include absorbance, fluorescence/phosphorescence, wavelength shift, phase modulation, bio/chemiluminescence, reflectance, light scattering, and refractive index.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, a sensor has a sensitivity (or slope) of from about 1 to about 100 picoAmps of current for every 1 mg/dL of glucose analyte.

The phrases and terms “small diameter sensor,” “small structured sensor,” and “micro-sensor” as used herein are broad phrases and terms, and are to be given their ordinary and customary meaning to a person of ordinary skill in the art (and are not to be limited to a special or customized meaning), and refer without limitation to sensing mechanisms that are less than about 2 mm in at least one dimension. In further examples, the sensing mechanisms are less than about 1 mm in at least one dimension. In some examples, the sensing mechanism (sensor) is less than about 0.95, 0.9, 0.85, 0.8, 0.75, 0.7, 0.65, 0.6, 0.5, 0.4, 0.3, 0.2, or 0.1 mm. In some examples, the maximum dimension of an independently measured length, width, diameter, thickness, or circumference of the sensing mechanism does not exceed about 2 mm. In some examples, the sensing mechanism is a needle-type sensor, wherein the diameter is less than about 1 mm, see, for example, U.S. Pat. No. 6,613,379 to Ward et al. and U.S. Pat. No. 7,497,827 to Brister et al., both of which are incorporated herein by reference in their entirety. In some alternate examples, the sensing mechanism includes electrodes deposited on a substantially planar substrate, wherein the thickness of the implantable portion is less than about 1 mm, see, for example U.S. Pat. No. 6,175,752 to Say et al. and U.S. Pat. No. 5,779,665 to Mastrototaro et. al., both of which are incorporated herein by reference in their entirety. Examples of methods of forming the sensors (sensor electrode layouts and membrane) and sensor systems discussed herein may be found in currently pending U.S. Pat. Pub. No. 2019-0307371, Boock et al., incorporated by reference in its entirety herein.

The phrase “soft segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts flexibility to the chain. The phrase “soft segment” can be further characterized as an amorphous material with a low T g, e.g., a Tg not typically higher than ambient temperature or normal mammalian body temperature.

The phrase “solid portions” as used herein is a broad term, and is to be given its ordinary and customary meaning to a person of ordinary skill in the art (and is not to be limited to a special or customized meaning), and refers without limitation to portions of a membrane's material having a mechanical structure that demarcates cavities, voids, or other non-solid portions.

The term and phrase “zwitterion” and “zwitterionic compound” as used herein are each a broad term and phrase, and are to be given their ordinary and customary meaning to a person of ordinary skill in the art (and is not to be limited to a special or customized meaning), and refer without limitation to compounds in which a neutral molecule of the compound has a unit positive and unit negative electrical charge at different locations within the molecule. Such compounds are a type of dipolar compound, and are also sometimes referred to as “inner salts.”

The phrases “zwitterion precursor” or “zwitterionic compound precursor” as used herein are broad phrases, and are to be given their ordinary and customary meaning to a person of ordinary skill in the art (and is not to be limited to a special or customized meaning), and refer without limitation to any compound that is not itself a zwitterion, but can become a zwitterion in a final or transition state through chemical reaction. In some examples described herein, devices comprise zwitterion precursors that can be converted to zwitterions prior to in vivo implantation of the device. Alternately, in some examples described herein, devices comprise zwitterion precursors that can be converted to zwitterions by some chemical reaction that occurs after in vivo implantation of the device. Such reactions are known to a person of ordinary skill in the art and include ring opening reaction, addition reaction such as Michael addition. This method is especially useful when the polymerization of betaine containing monomer is difficult due to technical challenges such as solubility of betaine monomer to achieve desired physical properties such as molecular weight and mechanical strength. Post-polymerization modification or conversion of betaine precursor can be a practical way to achieve desired polymer structure and composition. Examples of such as precursors include tertiary amines, quaternary amines, pyridines, and others detailed herein.

The phrases “zwitterion derivative” or “zwitterionic compound derivative” as used herein are broad phrases, and are to be given their ordinary and customary meaning to a person of ordinary skill in the art (and is not to be limited to a special or customized meaning), and refer without limitation to any compound that is not itself a zwitterion, but rather is the product of a chemical reaction where a zwitterion is converted to a non-zwitterion. Such reactions can be reversible, such that under certain conditions zwitterion derivatives can act as zwitterion precursors. For example, hydrolyzable betaine esters formed from zwitterionic betaines are cationic zwitterion derivatives that under the appropriate conditions are capable of undergoing hydrolysis to revert to zwitterionic betaines.

Devices and probes that are transcutaneously inserted or implanted into subcutaneous tissue conventionally elicit a foreign body response (FBR), which includes invasion of inflammatory cells that ultimately forms a foreign body capsule (FBC), as part of the body's response to the introduction of a foreign material. The continuous monitoring systems discussed herein include continuous analyte monitoring systems configured to monitor one, two, or more analytes concurrently, sequentially, and/or randomly (which is inclusive of events that can take place independently in picoseconds, nanoseconds, milliseconds, seconds, or minutes) to predict health-related events and health systems performance (e.g., the current and future performance of the human body's systems such as the circulatory, respiratory, digestive, or other systems or combinations of organs or systems). In examples, insertion or implantation of a device, for example, a glucose sensing device, can result in an acute inflammatory reaction resolving to chronic inflammation with concurrent building of fibrotic tissue, such as described in detail above. Eventually, over a period of time, a mature FBC, including primarily contractile fibrous tissue forms around the device. See Shanker and Greisler, Inflammation and Biomaterials in Greco RS, ed., “Implantation Biology: The Host Response and Biomedical Devices” pp 68-80, CRC Press (1994). The FBC surrounding conventional implanted devices has been shown to hinder or block the transport of analytes across the device-tissue interface. Thus, continuous extended life analyte transport (e.g., beyond the first few days) in vivo has been conventionally believed to be unreliable or impossible.

In some examples, certain aspects of the FBR in the first few days may play a role in noise. It has been observed that some sensors function more poorly during the first few hours after insertion than they do later. This is exemplified by noise and/or a suppression of the signal during the first few hours (e.g., about 2 to about 24 hours) after insertion. These anomalies often resolve spontaneously after which the sensors become less noisy, have improved sensitivity, and are more accurate than during the early period. It has been observed that some transcutaneous sensors and wholly implantable sensors are subject to noise for a period of time after application to the host (i.e., inserted transcutaneously or wholly implanted below the skin).

When a sensor is first inserted or implanted into the subcutaneous tissue, it comes into contact with a wide variety of possible tissue conformations. Subcutaneous tissue in different hosts may be relatively fat free in cases of very athletic people or may be mostly composed of fat in the majority of people. Fat comes in a wide array of textures from very white, puffy fat to very dense, fibrous fat. Some fat is very yellow and dense looking; some is very clear, puffy, and white looking, while in other cases it is more red or brown. The fat may be several inches thick or only 1 cm thick. It may be very vascular or relatively nonvascular. Many hosts with diabetes have some subcutaneous scar tissue due to years of insulin pump use or insulin injection. At times, during insertion, sensors may come to rest in such a scarred area. The subcutaneous tissue may even vary greatly from one location to another in the abdomen of a given host. Moreover, by chance, the sensor may come to rest near a more densely vascularized area or in a less vascularized area of a given host. While not wishing to be bound by theory, it is believed that creating a space between the sensor surface and the surrounding cells, including formation of a fluid pocket surrounding the sensor, may enhance sensor performance. Accordingly, the continuous analyte monitoring systems discussed herein provide an extended life without compromising accuracy, which can also improve the experience of the host.

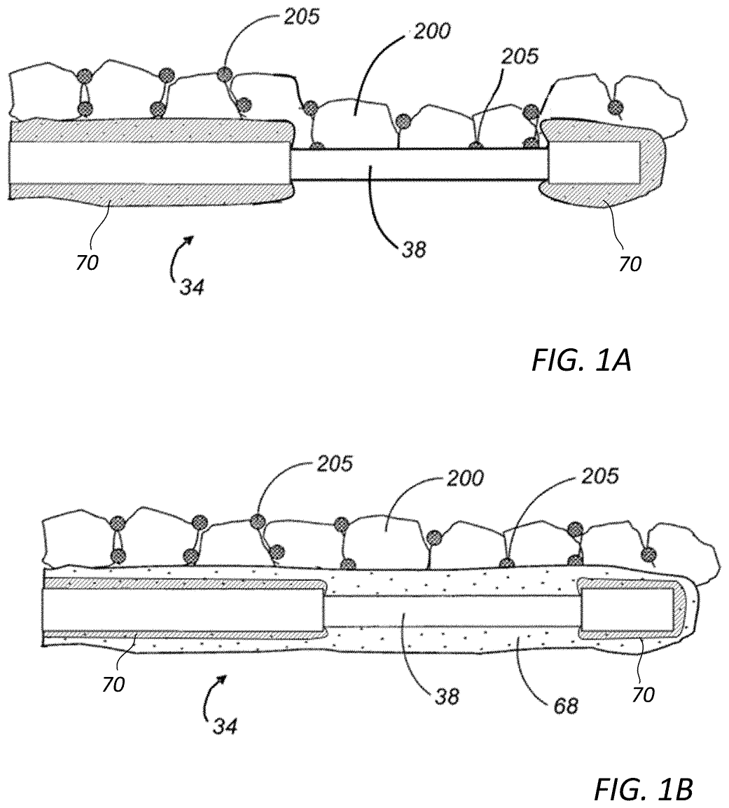

FIG. 1A is a side schematic view of adipose cell contact with an inserted transcutaneous sensor or an implanted sensor 34. In this case, the sensor 34 is firmly inserted into a small space with adipose cells pressing up against the surface. Close association of the adipose cells with the sensor can also occur, for example wherein the surface of the sensor is hydrophobic. For example, the adipose cells 200 and/or inflammatory cells and/or other tissues types such as dermis, muscle facia, and/or connective tissue may create an active metabolic interface that can physically block the surface of the sensor and/or access to a working electrode 38.

Typically adipose cells can be about 120 microns in diameter and are typically fed by tiny capillaries 205. When the sensor is pressed against the fat tissue, very few capillaries may actually come near the surface of the sensor. This may be analogous to covering the surface of the sensor with an impermeable material such as cellophane, for example. Even if there were a few small holes in the cellophane, the sensor's function would likely be compromised. Additionally, the surrounding tissue has a low metabolic rate and therefore does not require high amounts of glucose and oxygen. While not wishing to be bound by theory, it is believed that, during this early period, the sensor's signal can be noisy and the signal can be suppressed due to close association of the sensor surface with the adipose cells and decreased availability of oxygen and glucose both for physical-mechanical reasons and physiological reasons.

Referring now to extended function of a sensor, after a few days or weeks after implantation, these devices typically lose their function. In some applications, cellular attack or migration of cells to the sensor can cause reduced sensitivity and/or function of the device, particularly after the first day of implantation. See also, for example, U.S. Pat. No. 5,791,344 and Gross et al. and “Performance Evaluation of the MiniMed Continuous Monitoring System During Host home Use,” Diabetes Technology and Therapeutics, (2000) 2(1):49-56, which have reported a glucose oxidase-based device, approved for use in humans by the Food and Drug Administration, that functions well for several days following implantation but loses function quickly after the several days (e.g., a few days up to about 14 days).

Without being bound by any theory, it is believed that this diminished performance of device function is most likely due to cells, such as polymorphonuclear cells and monocytes that migrate to the sensor site during the first few days after implantation. These cells consume local glucose and oxygen, among other things. If there is an overabundance of such cells, they can deplete glucose and/or oxygen before it is able to reach the device enzyme layer, thereby reducing the sensitivity of the device or rendering it non-functional. Further inhibition of device function can be due to inflammatory cells, for example, macrophages, that associate, for example, align at the interface, with the implantable device and adjacent tissue, and physically block and/or attenuate the transport/flux of glucose into the device, for example, by formation of a barrier cell layer. Additionally, these inflammatory cells can biodegrade many artificial biomaterials (some of which were, until recently, considered non-biodegradable). When activated by a foreign body, tissue macrophages degranulate, releasing hypochlorite (bleach) and other oxidative species, enzymes, superperoxide anion, hydroxyl ion/radical generating moieties that are known to break down a variety of polymers.

FIG. 1B is a side schematic view of a biointerface membrane of an inserted transcutaneous sensor or an implanted sensor in one exemplary example. In this illustration, a biointerface membrane 68 surrounds the sensor 34, covering a working electrode 38. In one example, the biointerface membrane 68 is used in combination with a bioactive releasing membrane 70, where the bioactive releasing membrane is adjacent to or at least partially covers a portion of the biointerface membrane 68. In another example, the bioactive releasing membrane 70 is at least partially covered by the biointerface membrane 68. In another example, the bioactive releasing membrane 70 is used without the biointerface membrane 68.

Accordingly, a sensor including a biointerface, including but not limited to, for example, porous biointerface materials, mesh cages, and the like, all of which are described in more detail elsewhere herein, can be employed to improve sensor function (e.g., first few hours to days).

In some circumstances, for example in extended sensors, it is believed that that foreign body response is the dominant event surrounding extended implantation of an implanted device, and can be managed or manipulated to support rather than hinder or block analyte transport. In another aspect, in order to extend the lifetime of the sensor, one example employ materials that promote vascularized tissue ingrowth, for example within a porous biointerface membrane. For example, tissue in-growth into a porous biointerface material surrounding an extended sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous biointerface materials that surround the sensor and promote ingrowth of tissue and microvasculature over time.

### Sensing Mechanism

In general, the analyte sensors of the present disclosure include a sensing mechanism 36 with a small structure (e.g., small structured-, micro- or small diameter sensor), for example, a needle-type sensor, in at least a portion thereof. As used herein a “small structure” preferably refers to an architecture with at least one dimension less than about 1 mm. The small structured sensing mechanism can be wire-based substrate, substrate based, or any other architecture. In some alternative examples, the term “small structure” can also refer to slightly larger structures, such as those having their smallest dimension being greater than about 1 mm, however, the architecture (e.g., mass or size) is designed to minimize the foreign body response due to size and/or mass. In one example, a biointerface membrane is formed onto the sensing mechanism 36 as described in more detail below. In another example, a bioactive releasing membrane 70 is formed on sensing mechanism 36, adjacent to working electrode 38. In another example, the bioactive releasing membrane 70 is used in combination with the biointerface layer 68. In another example, the bioactive releasing membrane 70 is used without the biointerface layer 68.

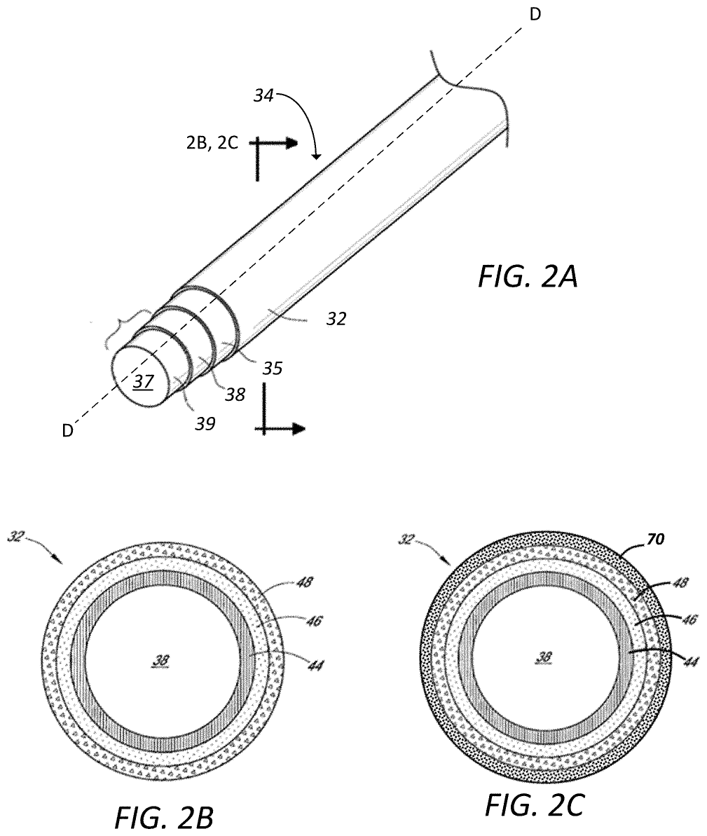

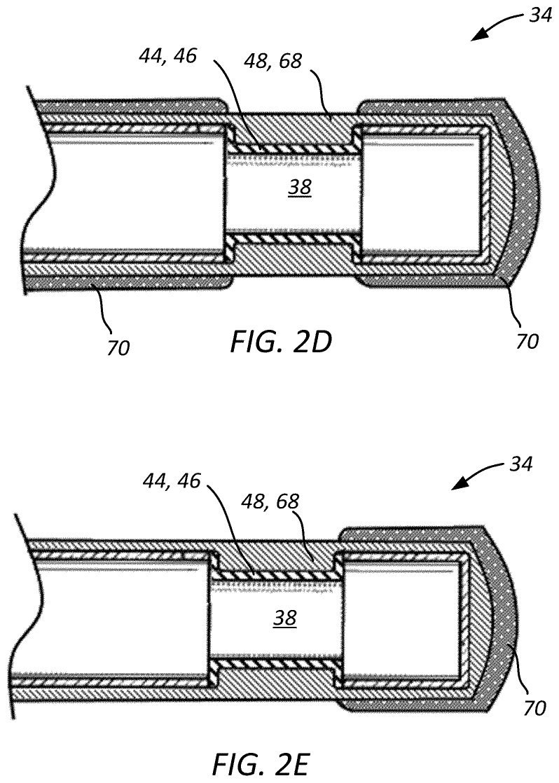

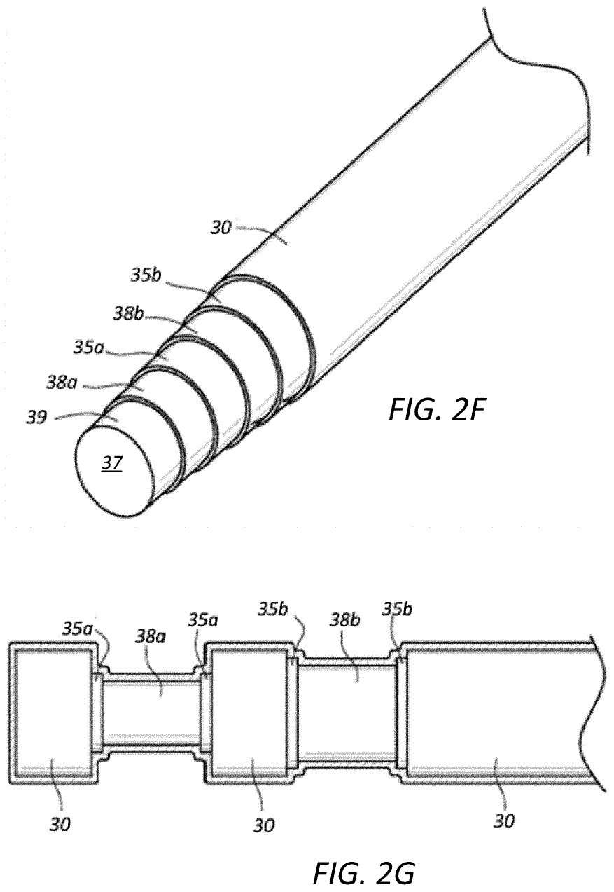

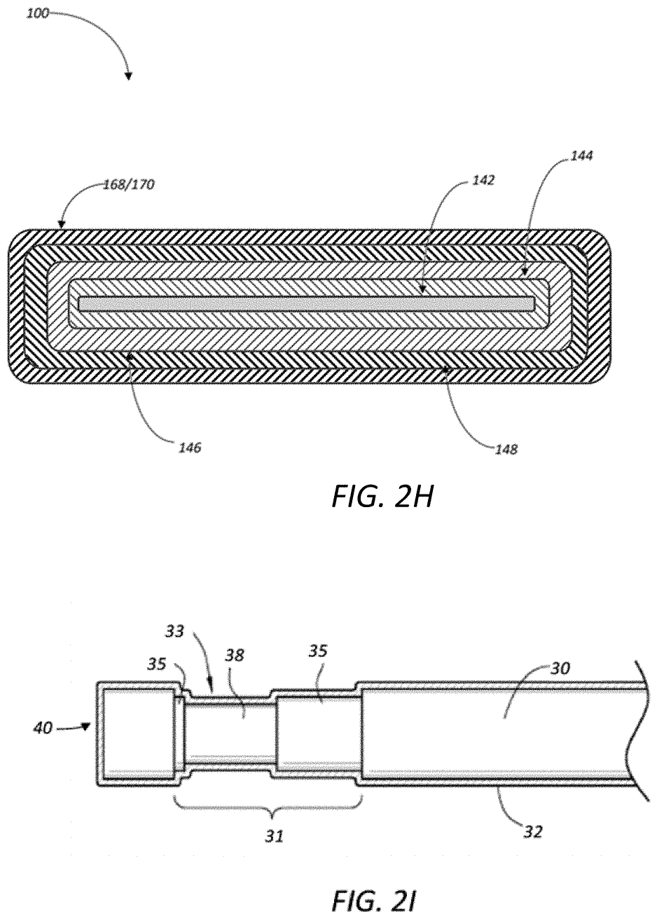

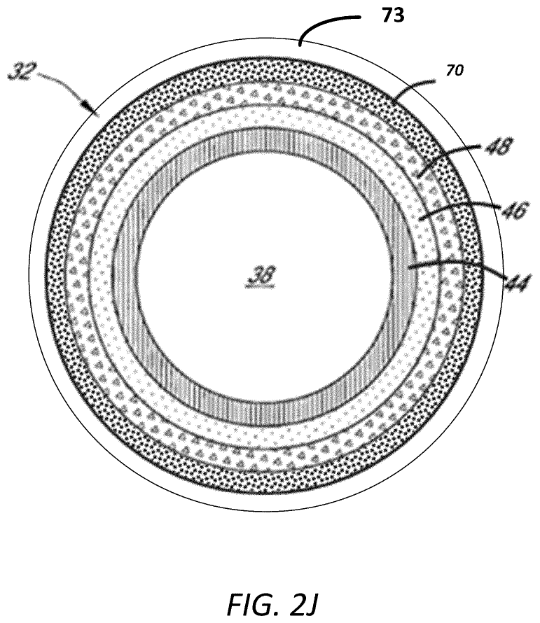

FIG. 2A is an expanded view of an exemplary example of a continuous analyte sensor 34, also referred to as a transcutaneous analyte sensor, or needle-type sensor, particularly illustrating the sensing mechanism 36. Preferably, the sensing mechanism comprises a small structure as defined herein and is adapted for insertion under the host's skin, and the remaining body of the sensor (e.g., electronics, etc.) can reside ex vivo. In the illustrated example, the continuous analyte sensor 34, includes two electrodes, i.e., a working electrode 38 and at least one additional electrode, which may function as a counter and/or reference electrode 30, hereinafter referred to as the reference electrode 30.

In some exemplary examples, each electrode is formed from a fine wire with a diameter of from about 0.001 or less to about 0.010 inches or more, for example, and is formed from, e.g., a plated insulator, a plated wire, or bulk electrically conductive material. Although the illustrated electrode configuration and associated text describe one preferred method of forming a transcutaneous sensor, a variety of known transcutaneous sensor configurations can be employed with the transcutaneous analyte sensor system of the present disclosure, such as are described in U.S. Pat. No. 6,695,860 to Ward et al., U.S. Pat. No. 6,565,509 to Say et al., U.S. Pat. No. 6,248,067 to Causey III et al., and U.S. Pat. No. 6,514,718 to Heller et al.

In examples, the working electrode comprises a wire formed from a conductive material, such as platinum, platinum-iridium, palladium, graphite, gold, carbon, conductive polymer, alloys, or the like. Although the electrodes can by formed by a variety of manufacturing techniques (bulk metal processing, deposition of metal onto a substrate, or the like), it can be advantageous to form the electrodes from plated wire (e.g., platinum on steel wire) or bulk metal (e.g., platinum wire). It is believed that electrodes formed from bulk metal wire provide superior performance (e.g., in contrast to deposited electrodes), including increased stability of assay, simplified manufacturability, resistance to contamination (e.g., which can be introduced in deposition processes), and improved surface reaction (e.g., due to purity of material) without peeling or delamination.

The working electrode 38 is configured to measure the concentration of one or more analytes. In an enzymatic electrochemical sensor for detecting glucose, for example, the working electrode measures the hydrogen peroxide produced by an enzyme catalyzed reaction of the analyte being detected and creates a measurable electronic current. For example, in the detection of glucose wherein glucose oxidase produces hydrogen peroxide as a byproduct, hydrogen peroxide reacts with the surface of the working electrode producing two protons (2H+), two electrons (2e⁻) and one molecule of oxygen (O₂), which produces the electronic current being detected.

The working electrode 38 is covered with an insulating material, for example, a non-conductive polymer. Dip-coating, spray-coating, vapor-deposition, or other coating or deposition techniques can be used to deposit the insulating material on the working electrode. In one example, the insulating material comprises parylene, which can be an advantageous polymer coating for its strength, lubricity, and electrical insulation properties. Generally, parylene is produced by vapor deposition and polymerization of para-xylylene (or its substituted derivatives). However, any suitable insulating material can be used, for example, fluorinated polymers, polyethyleneterephthalate, polyurethane, polyimide, other nonconducting polymers, or the like. Glass or ceramic materials can also be employed. Other materials suitable for use include surface energy modified coating systems such as are marketed under the trade names AMC18, AMC148, AMC141, and AMC321 by Advanced Materials Components Express of Bellafonte, Pa. In some alternative examples, however, the working electrode may not require a coating of insulator.

Preferably, the reference electrode 30, which may function as a reference electrode alone, or as a dual reference and counter electrode, is formed from silver, silver/silver chloride, or the like. Preferably, the electrodes are juxtapositioned and/or twisted with or around each other; however other configurations are also possible. In one example, the reference electrode 30 is helically wound around the working electrode 38 as illustrated in FIG. 1B. The assembly of wires may then be optionally coated together with an insulating material, similar to that described above, in order to provide an insulating attachment (e.g., securing together of the working and reference electrodes).

In examples wherein an outer insulator 35 is disposed, a portion of the coated assembly structure can be stripped or otherwise removed, for example, by hand, excimer lasing, chemical etching, laser ablation, grit-blasting (e.g., with sodium bicarbonate, solid carbon dioxide, or other suitable grit), or the like, to expose the electrochemically active surfaces. Alternatively, a portion of the electrode can be masked prior to depositing the insulator in order to maintain an exposed electrochemically active surface area. In one exemplary example, grit blasting is implemented to expose the electrochemically active surfaces, preferably utilizing a grit material that is sufficiently hard to ablate the polymer material, while being sufficiently soft so as to minimize or avoid damage to the underlying metal electrode (e.g., a platinum electrode). Although a variety of “grit” materials can be used (e.g., sand, talc, walnut shell, ground plastic, sea salt, solid carbon dioxide, and the like), in some one example, sodium bicarbonate is an advantageous grit-material because it is sufficiently hard to ablate, e.g., a parylene coating without damaging, e.g., an underlying platinum conductor. One additional advantage of sodium bicarbonate blasting includes its polishing action on the metal as it strips the polymer layer, thereby eliminating a cleaning step that might otherwise be necessary.

In some examples, a radial window is formed through the insulating material to expose a circumferential electrochemically active surface of the working electrode. Additionally, sections of electrochemically active surface of the reference electrode are exposed. For example, the sections of electrochemically active surface can be masked during deposition of an outer insulating layer or etched after deposition of an outer insulating layer.

In some applications, cellular attack or migration of cells to the sensor can cause reduced sensitivity and/or function of the device, particularly after the first day of implantation. However, when the exposed electroactive surface is distributed circumferentially about the sensor (e.g., as in a radial window), the available surface area for reaction can be sufficiently distributed so as to minimize the effect of local cellular invasion of the sensor on the sensor signal. Alternatively, a tangential exposed electrochemically active window can be formed, for example, by stripping only one side of the coated assembly structure. In other alternative examples, the window can be provided at the tip of the coated assembly structure such that the electrochemically active surfaces are exposed at the tip of the sensor. Other methods and configurations for exposing electrochemically active surfaces can also be employed.

Preferably, the above-exemplified sensor has an overall diameter of not more than about 0.020 inches (about 0.51 mm), more preferably not more than about 0.018 inches (about 0.46 mm), and most preferably not more than about 0.016 inches (0.41 mm). In some examples, the working electrode has a diameter of from about 0.001 inches or less to about 0.010 inches or more, preferably from about 0.002 inches to about 0.008 inches, and more preferably from about 0.004 inches to about 0.005 inches, including all ranges and subranges therebetween. The length of the window can be from about 0.1 mm (about 0.004 inches) or less to about 2 mm (about 0.078 inches) or more, and preferably from about 0.5 mm (about 0.02 inches) to about 0.75 mm (0.03 inches), including all ranges and subranges therebetween. In such examples, the exposed surface area of the working electrode is preferably from about 0.000013 in2 (0.0000839 cm2) or less to about 0.0025 in2(0.016129 cm2) or more (assuming a diameter of from about 0.001 inches to about 0.010 inches and a length of from about 0.004 inches to about 0.078 inches), including all ranges and subranges therebetween. The exposed surface area of the working electrode is selected to produce an analyte signal with a current in the femtoampere range, picoampere range, the nanoampere range, the or the microampere range such as is described in more detail elsewhere herein. However, a current in the picoampere range or less can be dependent upon a variety of factors, for example the electronic circuitry design (e.g., sample rate, current draw, A/D converter bit resolution, etc.), the membrane system (e.g., permeability of the analyte through the membrane system), and the exposed surface area of the working electrode. Accordingly, the exposed electrochemically active working electrode surface area can be selected to have a value greater than or less than the above-described ranges taking into consideration alterations in the membrane system and/or electronic circuitry. In examples of a glucose sensor, it can be advantageous to minimize the surface area of the working electrode while maximizing the diffusivity of glucose in order to optimize the signal-to-noise ratio while maintaining sensor performance in both high and low glucose concentration ranges.

In some alternative examples, the exposed surface area of the working (and/or other) electrode can be increased by altering the cross-section of the electrode itself. For example, in some examples the cross-section of the working electrode can be defined by a cross, star, cloverleaf, ribbed, dimpled, ridged, irregular, or other non-circular configuration; thus, for any predetermined length of electrode, a specific increased surface area can be achieved (as compared to the area achieved by a circular cross-section). Increasing the surface area of the working electrode can be advantageous in providing an increased signal responsive to the analyte concentration, which in turn can be helpful in improving the signal-to-noise ratio, for example.

In some alternative examples, additional electrodes can be included within the assembly, for example, a three-electrode system (working, reference, and counter electrodes) and/or an additional working electrode (e.g., an electrode which can be used to generate oxygen, which is configured as a baseline subtracting electrode, or which is configured for measuring additional analytes). U.S. Pat. No. 7,081,195, filed Dec. 7, 2004 and entitled “SYSTEMS AND METHODS FOR IMPROVING ELECTROCHEMICAL ANALYTE SENSORS” and U.S. Pat. No. 7,715,893, filed Dec. 3, 2004 and entitled “CALIBRATION TECHNIQUES FOR A CONTINUOUS ANALYTE SENSOR” describe some systems and methods for implementing and using additional working, counter, and/or reference electrodes. In one implementation wherein the sensor comprises two working electrodes, the two working electrodes are juxtapositioned (e.g., extend parallel to each other), around which the reference electrode is disposed (e.g., helically wound). In some examples wherein two or more working electrodes are provided, the working electrodes can be formed in a double-, triple-, quad-, etc. helix configuration along the length of the sensor (for example, surrounding a reference electrode, insulated rod, or other support structure). The resulting electrode system can be configured with an appropriate membrane system, wherein the first working electrode is configured to measure a first signal comprising glucose and baseline and the additional working electrode is configured to measure a baseline signal consisting of baseline only (e.g., configured to be substantially similar to the first working electrode without an enzyme disposed thereon). In this way, the baseline signal can be subtracted from the first signal to produce a glucose-only signal that is substantially not subject to fluctuations in the baseline and/or interfering species on the signal. Accordingly, the above-described dimensions can be altered as desired. Although the present disclosure discloses one electrode configuration including one bulk metal wire helically wound around another bulk metal wire, other electrode configurations are also contemplated. In an alternative example, the working electrode comprises a tube with a reference electrode disposed or coiled inside, including an insulator there between. Alternatively, the reference electrode comprises a tube with a working electrode disposed or coiled inside, including an insulator there between. In another alternative example, a polymer (e.g., insulating) rod is provided, wherein the electrodes are deposited (e.g., electro-plated) thereon. In yet another alternative example, a metallic (e.g., steel) rod is provided, coated with an insulating material, onto which the working and reference electrodes are deposited. In yet another alternative example, one or more working electrodes are helically wound around a reference electrode.

While the methods of the present disclosure are especially well suited for use with small structured-, micro- or small diameter sensors, the methods can also be suitable for use with larger diameter sensors, e.g., sensors of 1 mm to about 2 mm or more in diameter.

In some alternative examples, the sensing mechanism includes electrodes deposited on a planar substrate, wherein the thickness of the implantable portion is less than about 1 mm, see, for example U.S. Pat. No. 6,175,752, to Say et al. and U.S. Pat. No. 5,779,665, to Mastrototaro et al., both of which are incorporated herein by reference in their entirety.

### Sensing Membranes

In examples, a sensing membrane 32 is disposed over the electrochemically active surfaces of the continuous analyte sensor 34 and includes one or more domains or layers. In general, the sensing membrane functions to control the flux of a biological fluid there through and/or to protect sensitive regions of the sensor from contamination by the biological fluid, for example. Some conventional electrochemical enzyme-based analyte sensors generally include a sensing membrane that controls the flux of the analyte being measured, protects the electrodes from contamination of the biological fluid, and/or provides an enzyme that catalyzes the reaction of the analyte with a co-factor, for example. See, e.g., U.S. Pat. Pub. No. 2005/0245799, filed May 3, 2004 entitled “IMPLANTABLE ANALYTE SENSOR” and U.S. Pat. No. 7,497,827, filed Mar. 10, 2005 and entitled “TRANSCUTANEOUS ANALYTE SENSOR,” each of which are incorporated herein by reference in their entirety.

The sensing membranes of the present disclosure can include any membrane configuration suitable for use with any analyte sensor (such as described in more detail above). In general, the sensing membranes of the present disclosure include one or more domains, all or some of which can be adhered to or deposited on the analyte sensor as is appreciated by one skilled in the art. In examples, the sensing membrane generally provides one or more of the following functions: 1) protection of the exposed electrode surface from the biological environment, 2) diffusion resistance (limitation) of the analyte, 3) a catalyst for enabling an enzymatic reaction, 4) limitation or blocking of interfering species, and 5) hydrophilicity at the electrochemically reactive surfaces of the sensor interface, such as described in the above-referenced U.S. patent publications.

### Electrode Domain

In some examples, the membrane system comprises an optional an electrode membrane comprising an electrode domain. The electrode domain is provided to ensure that an electrochemical reaction occurs between the electrochemically active surfaces of the working electrode and the reference electrode, and thus the electrode domain is preferably situated more proximal to the electrochemically active surfaces than the enzyme domain. Preferably, the electrode domain includes a semipermeable coating that maintains a layer of water at the electrochemically reactive surfaces of the sensor, for example, a humectant in a binder material can be employed as an electrode domain; this allows for the full transport of ions in the aqueous environment. The electrode domain can also assist in stabilizing the operation of the sensor by overcoming electrode start-up and drifting problems caused by inadequate electrolyte. The material that forms the electrode domain can also protect against pH-mediated damage that can result from the formation of a large pH gradient due to the electrochemical activity of the electrodes.

In examples, the electrode domain includes a flexible, water-swellable, hydrogel film having a “dry film” thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns, including all ranges and subranges therebetween. “Dry film” thickness refers to the thickness of a cured film cast from a coating formulation by standard coating techniques.

In certain examples, the electrode domain is formed of a curable mixture of a urethane polymer and a hydrophilic polymer. Particularly preferred coatings are formed of a polyurethane polymer having carboxylate functional groups and non-ionic hydrophilic polyether segments, wherein the polyurethane polymer is crosslinked with a water soluble carbodiimide (e.g., 1-ethyl-3-(3-dimethylaminopropyl)carbodiimide (EDC))) in the presence of polyvinylpyrrolidone and cured at a moderate temperature of about 50° C.

Preferably, the electrode domain is deposited by spray or dip-coating the electrochemically active surfaces of the sensor. More preferably, the electrode domain is formed by dip-coating the electrochemically active surfaces in an electrode solution and curing the domain for a time of from about 15 to about 30 minutes at a temperature of from about 40 to about 55° C. (and can be accomplished under vacuum (e.g., 20 to 30 mmHg)), including all ranges and subranges therebetween. In examples wherein dip-coating is used to deposit the electrode domain, a preferred insertion rate of from about 1 to about 3 inches per minute, with a preferred dwell time of from about 0.5 to about 2 minutes, and a preferred withdrawal rate of from about 0.25 to about 2 inches per minute provide a functional coating, including all ranges and subranges therebetween. However, values outside of those set forth above can be acceptable or even desirable in certain examples, for example, dependent upon viscosity and surface tension as is appreciated by one skilled in the art. In one example, the electrochemically active surfaces of the electrode system are dip-coated one time (one layer) and cured at 50° C. under vacuum for 20 minutes.

Although an independent electrode domain is described herein, in some examples, sufficient hydrophilicity can be provided in the interference domain and/or enzyme domain (the domain adjacent to the electrochemically active surfaces) so as to provide for the full transport of ions in the aqueous environment (e.g. without a distinct electrode domain).

### Interference Domain

In some examples, an optional interference domain is provided, which generally includes a polymer domain that restricts the flow of one or more interferants. In some examples, the interference domain functions as a molecular sieve that allows analytes and other substances that are to be measured by the electrodes to pass through, while preventing passage of other substances, including interferants such as ascorbate and urea (see U.S. Pat. No. 6,001,067 to Shults). Some known interferants for a glucose-oxidase based electrochemical sensor include acetaminophen, ascorbic acid, bilirubin, cholesterol, creatinine, dopamine, ephedrine, ibuprofen, L-dopa, methyldopa, salicylate, tetracycline, tolazamide, tolbutamide, triglycerides, and uric acid.

Several polymer types that can be utilized as a base material for the interference domain include polyurethanes, polymers having pendant ionic groups, and polymers having controlled pore size, for example. In one example, the interference domain includes a thin, hydrophobic membrane that is non-swellable and restricts diffusion of low molecular weight species. The interference domain is permeable to relatively low molecular weight substances, such as hydrogen peroxide, but restricts the passage of higher molecular weight substances, including glucose and ascorbic acid. Other systems and methods for reducing or eliminating interference species that can be applied to the membrane system of the present disclosure are described in U.S. Pat. No. 7,074,307 filed Jul. 21, 2004 and entitled “ELECTRODE SYSTEMS FOR ELECTROCHEMICAL SENSORS,” U.S. Pat. Pub. No. 2005/0176136, filed Nov. 16, 2004 and entitled, “AFFINITY DOMAIN FOR AN ANALYTE SENSOR,” U.S. Pat. No. 7,081,195, filed Dec. 7, 2004 and entitled “SYSTEMS AND METHODS FOR IMPROVING ELECTROCHEMICAL ANALYTE SENSORS” and U.S. Pat. No. 7,715,893, filed Dec. 3, 2004 and entitled, “CALIBRATION TECHNIQUES FOR A CONTINUOUS ANALYTE SENSOR.” In some alternative examples, a distinct interference domain is not included.

In examples, the interference domain is deposited onto the electrode domain (or directly onto the electrochemically active surfaces when a distinct electrode domain is not included) for a domain thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns, including all ranges and subranges therebetween. Thicker membranes can also be useful, but thinner membranes are generally preferred because they have a lower impact on the rate of diffusion of hydrogen peroxide from the enzyme membrane to the electrodes. Unfortunately, the thin thickness of the interference domains conventionally used can introduce variability in the membrane system processing. For example, if too much or too little interference domain is incorporated within a membrane system, the performance of the membrane can be adversely affected.

### Enzyme Domain

In one example, the membrane system further includes an enzyme domain disposed more distally from the electrochemically active surfaces than the interference domain (or electrode domain when a distinct interference is not included). In some examples, the enzyme domain is directly deposited onto the electrochemically active surfaces (when neither an electrode or interference domain is included). In one example, the enzyme domain provides an enzyme to catalyze the reaction of the analyte and its co-reactant, as described in more detail below. Preferably, the enzyme domain includes glucose oxidase; however other oxidases, for example, galactose oxidase, lactate oxidase, or uricase oxidase, can also be used.

For an enzyme-based electrochemical glucose sensor to perform well, the sensor's response is preferably limited by neither enzyme activity nor co-reactant concentration. Because enzymes, including glucose oxidase, are subject to deactivation as a function of time even in ambient conditions, this behavior is compensated for in forming the enzyme domain. Preferably, the enzyme domain is constructed of aqueous dispersions of colloidal polyurethane polymers including the enzyme. However, in alternative examples the enzyme domain is constructed from an oxygen enhancing material, for example, silicone, or fluorocarbon, in order to provide a supply of excess oxygen during transient ischemia. Preferably, the enzyme is immobilized within the domain. See U.S. Pat. No. 7,379,765 filed on Jul. 21, 2004 and entitled “Oxygen Enhancing Membrane Systems for Implantable Device.”

In examples, the enzyme domain is deposited onto the interference domain for a domain thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns, including all ranges and subranges therebetween. However in some examples, the enzyme domain is deposited onto the electrode domain or directly onto the electrochemically active surfaces. Preferably, the enzyme domain is deposited by spray or dip coating. More preferably, the enzyme domain is formed by dip-coating the electrode domain into an enzyme domain solution and curing the domain for from about 15 to about 30 minutes at a temperature of from about 40 to about 55° C. (and can be accomplished under vacuum (e.g., 20 to 30 mmHg)), including all ranges and subranges therebetween. In examples wherein dip-coating is used to deposit the enzyme domain at room temperature, a preferred insertion rate of from about 1 inch per minute to about 3 inches per minute, with a preferred dwell time of from about 0.5 minutes to about 2 minutes, and a preferred withdrawal rate of from about 0.25 inch per minute to about 2 inches per minute provide a functional coating, including all ranges and subranges therebetween. However, values outside of those set forth above can be acceptable or even desirable in certain examples, for example, dependent upon viscosity and surface tension as is appreciated by one skilled in the art. In one example, the enzyme domain is formed by dip coating two times (namely, forming two layers) in a coating solution and curing at 50° C. under vacuum for 20 minutes. However, in some examples, the enzyme domain can be formed by dip-coating and/or spray-coating one or more layers at a predetermined concentration of the coating solution, insertion rate, dwell time, withdrawal rate, and/or desired thickness.

### Resistance Domain

In one example, the membrane system includes a resistance domain disposed more distal from the electrochemically active surfaces than the enzyme domain. Although the following description is directed to a resistance domain for a glucose sensor, the resistance domain can be modified for other analytes and co-reactants as well.

There exists a molar excess of glucose relative to the amount of oxygen in blood; that is, for every free oxygen molecule in extracellular fluid, there are typically more than 100 glucose molecules present (see Updike et al., Diabetes Care 5:207-21(1982)). However, an immobilized enzyme-based glucose sensor employing oxygen as co-reactant is preferably supplied with oxygen in non-rate-limiting excess in order for the sensor to respond linearly to changes in glucose concentration, while not responding to changes in oxygen concentration. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 40 mg/dL. However, in a clinical setting, a linear response to glucose levels is desirable up to at least about 400 mg/dL.

The resistance domain includes a semi-permeable membrane that controls the flux of oxygen and glucose to the underlying enzyme domain, preferably rendering oxygen in a non-rate-limiting excess. As a result, the upper limit of linearity of glucose measurement is extended to a much higher value than that which is achieved without the resistance domain. In examples, the resistance domain exhibits an oxygen to glucose permeability ratio of from about 50:1 or less to about 400:1 or more, preferably about 200:1, including all ranges and subranges therebetween. As a result, one-dimensional reactant diffusion is adequate to provide excess oxygen at all reasonable glucose and oxygen concentrations found in the subcutaneous matrix (See Rhodes et al., Anal. Chem., 66:1520-1529 (1994)).

In alternative examples, a lower ratio of oxygen-to-glucose can be sufficient to provide excess oxygen by using a high oxygen solubility domain (for example, a silicone or fluorocarbon-based material or domain) to enhance the supply/transport of oxygen to the enzyme domain. If more oxygen is supplied to the enzyme, then more glucose can also be supplied to the enzyme without creating an oxygen rate-limiting excess. In alternative examples, the resistance domain is formed from a silicone composition, such as is described in U.S. Pat. Pub. No. 2005/0090607, filed Oct. 28, 2003 and entitled, “SILICONE COMPOSITION FOR BIOCOMPATIBLE MEMBRANE.”

In a preferred example, the resistance domain includes a polyurethane membrane with both hydrophilic and hydrophobic regions to control the diffusion of glucose and oxygen to an analyte sensor, the membrane being fabricated easily and reproducibly from commercially available materials. A suitable hydrophobic polymer component is a polyurethane, or polyetherurethaneurea. Polyurethane is a polymer produced by the condensation reaction of a diisocyanate and a difunctional hydroxyl-containing material. A polyurethaneurea is a polymer produced by the condensation reaction of a diisocyanate and a difunctional amine-containing material. In some examples, diisocyanates include aliphatic diisocyanates containing from about 4 to about 8 methylene units. Diisocyanates containing cycloaliphatic moieties can also be useful in the preparation of the polymer and copolymer components of the membranes of the present disclosure. The material that forms the basis of the hydrophobic matrix of the resistance domain can be any of those known in the art as appropriate for use as membranes in continuous analyte sensor devices and as having sufficient permeability to allow relevant compounds to pass through it, for example, to allow an oxygen molecule to pass through the membrane from the sample under examination in order to reach the active enzyme or electrochemical electrodes. Examples of materials which can be used to make non-polyurethane type membranes include vinyl polymers, polyethylene vinyl acetate copolymers, polyethers, polyalkylcarbonate, polycarbonates, polyalkylesters, polyesters, polyamides, inorganic polymers such as polysiloxanes and polycarbosiloxanes, natural polymers such as cellulosic and protein-based materials, and mixtures or combinations thereof.

In a preferred example, the hydrophilic polymer component of the resistance domain is polyethylene oxide. For example, one useful hydrophobic-hydrophilic copolymer component is a polyurethane polymer that includes about 20% hydrophilic polyethylene oxide. The polyethylene oxide portions of the copolymer are thermodynamically driven to separate from the hydrophobic portions of the copolymer and the hydrophobic polymer component. The 20% polyethylene oxide-based soft segment portion of the copolymer used to form the final blend affects the water pick-up and subsequent glucose permeability of the membrane.

In examples, the resistance domain is deposited onto the enzyme domain to yield a domain thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns, including all ranges and subranges therebetween. Preferably, the resistance domain is deposited onto the enzyme domain by spray coating or dip coating. In certain examples, spray coating is the preferred deposition technique. The spraying process atomizes and mists the solution, and therefore most or all of the solvent is evaporated prior to the coating material settling on the underlying domain, thereby minimizing contact of the solvent with the enzyme. One additional advantage of spray-coating the resistance domain as described in the present disclosure includes formation of a membrane system that substantially blocks or resists ascorbate (a known electrochemical interferant in hydrogen peroxide-measuring glucose sensors). While not wishing to be bound by theory, it is believed that during the process of depositing the resistance domain as described in the present disclosure, a structural morphology is formed, characterized in that ascorbate does not substantially permeate there through.

In examples, the resistance domain is deposited on the enzyme domain by spray-coating a solution of from about 1 wt. % to about 5 wt. % polymer and from about 95 wt. % to about 99 wt. % solvent, including all ranges and subranges therebetween. In spraying a solution of resistance domain material, including a solvent, onto the enzyme domain, it is desirable to mitigate or substantially reduce any contact with enzyme of any solvent in the spray solution that can deactivate the underlying enzyme of the enzyme domain. Tetrahydrofuran (THF) is one solvent that minimally or negligibly affects the enzyme of the enzyme domain upon spraying. Other solvents can also be suitable for use, as is appreciated by one skilled in the art.

Although a variety of spraying or deposition techniques can be used, spraying the resistance domain material and rotating the sensor at least one time by 180° can provide adequate coverage by the resistance domain. Spraying the resistance domain material and rotating the sensor at least two times by 120 degrees provides even greater coverage (one layer of 360° coverage), thereby ensuring resistivity to glucose, such as is described in more detail above.

In examples, the resistance domain is spray-coated and subsequently cured for a time of from about 15 to about 90 minutes at a temperature of from about 40 to about 60° C. (and can be accomplished under vacuum (e.g., 20 to 30 mmHg)), including all ranges and subranges therebetween. A cure time of up to about 90 minutes or more can be advantageous to ensure complete drying of the resistance domain. While not wishing to be bound by theory, it is believed that complete drying of the resistance domain aids in stabilizing the sensitivity of the glucose sensor signal. It reduces drifting of the signal sensitivity over time, and complete drying is believed to stabilize performance of the glucose sensor signal in lower oxygen environments.

In examples, the resistance domain is formed by spray-coating at least six layers (namely, rotating the sensor seventeen times by 120° for at least six layers of 360° coverage) and curing at 50° C. under vacuum for 60 minutes. However, the resistance domain can be formed by dip-coating or spray-coating any layer or plurality of layers, depending upon the concentration of the solution, insertion rate, dwell time, withdrawal rate, and/or the desired thickness of the resulting film.

Advantageously, sensors with the membrane system of the present disclosure, including an electrode domain and/or interference domain, an enzyme domain, and a resistance domain, provide stable signal response to increasing glucose levels of from about 40 to about 400 mg/dL, including all ranges and subranges therebetween, and sustained function (at least 90% signal strength) even at low oxygen levels (for example, at about 0.6 mg/L O₂). While not wishing to be bound by theory, it is believed that the resistance domain provides sufficient resistivity, or the enzyme domain provides sufficient enzyme, such that oxygen limitations are seen at a much lower concentration of oxygen as compared to prior art sensors.

In examples, a sensor signal with a current in the picoampere range or less is provided, which is described in more detail elsewhere herein. However, the ability to produce a signal with a current in the picoampere range can be dependent upon a combination of factors, including the electronic circuitry design (e.g., A/D converter, bit resolution, and the like), the membrane system (e.g., permeability of the analyte through the resistance domain, enzyme concentration, and/or electrolyte availability to the electrochemical reaction at the electrodes), and the exposed surface area of the working electrode. For example, the resistance domain can be designed to be more or less restrictive to the analyte depending upon to the design of the electronic circuitry, membrane system, and/or exposed electrochemically active surface area of the working electrode.

Accordingly, in examples, the membrane system is designed with a sensitivity of from about 1 pA/mg/dL to about 100 pA/mg/dL, preferably from about 5 pA/mg/dL to 25 pA/mg/dL, and more preferably from about 4 to about 7 pA/mg/dL, including all ranges and subranges therebetween. While not wishing to be bound by any particular theory, it is believed that membrane systems designed with a sensitivity in the preferred ranges permit measurement of the analyte signal in low analyte and/or low oxygen situations. Namely, conventional analyte sensors have shown reduced measurement accuracy in low analyte ranges due to lower availability of the analyte to the sensor and/or have shown increased signal noise in high analyte ranges due to insufficient oxygen necessary to react with the amount of analyte being measured. While not wishing to be bound by theory, it is believed that the membrane systems of the present disclosure, in combination with the electronic circuitry design and exposed electrochemical reactive surface area design, support measurement of the analyte in the picoampere range or less, which enables an improved level of resolution and accuracy in both low and high analyte ranges not seen in the prior art.

Although sensors of some examples described herein include an optional interference domain in order to block or reduce one or more interferants, sensors with the membrane system of the present disclosure, including an electrode domain, an enzyme domain, and a resistance domain, have been shown to inhibit ascorbate without an additional interference domain. Namely, the membrane system of the present disclosure, including an electrode domain, an enzyme domain, and a resistance domain, has been shown to be substantially non-responsive to ascorbate in physiologically acceptable ranges. While not wishing to be bound by theory, it is believed that the process of depositing the resistance domain by spray coating, as described herein, results in a structural morphology that is substantially resistance resistant to ascorbate.

### Interference-Domain Free Membrane Systems

In general, it is believed that appropriate solvents and/or deposition methods can be chosen for one or more of the domains of the membrane system that form one or more transitional domains such that interferants do not substantially permeate there through. Thus, sensors can be built without distinct or deposited interference domains, which are non-responsive to interferants. While not wishing to be bound by theory, it is believed that a simplified multilayer membrane system, more robust multilayer manufacturing process, and reduced variability caused by the thickness and associated oxygen and glucose sensitivity of the deposited micron-thin interference domain can be provided. Additionally, the optional polymer-based interference domain, which usually inhibits hydrogen peroxide diffusion, is eliminated, thereby enhancing the amount of hydrogen peroxide that passes through the membrane system.

### Oxygen Conduit

As described above, certain sensors depend upon an enzyme within the membrane system through which the host's bodily fluid passes and in which the analyte (for example, glucose) within the bodily fluid reacts in the presence of a co-reactant (for example, oxygen) to generate a product. The product is then measured using electrochemical methods, and thus the output of an electrode system functions as a measure of the analyte. For example, when the sensor is a glucose oxidase based glucose sensor, the species measured at the working electrode is H₂O₂. An enzyme, glucose oxidase, catalyzes the conversion of oxygen and glucose to hydrogen peroxide and gluconate according to the following reaction: Glucose+O2→Gluconate+H2O2

Because for each glucose molecule reacted there is a proportional change in the product, H₂O₂, one can monitor the change in H₂O₂ to determine glucose concentration. Oxidation of H₂O₂ by the working electrode is balanced by reduction of ambient oxygen, enzyme generated H₂O₂ and other reducible species at a counter electrode, for example. See Fraser, D. M., “An Introduction to In vivo Biosensing: Progress and Problems.” In “Biosensors and the Body,” D. M. Fraser, ed., 1997, pp. 1-56 John Wiley and Sons, New York))

In vivo, glucose concentration is generally about one hundred times or more that of the oxygen concentration. Consequently, oxygen is a limiting reactant in the electrochemical reaction, and when insufficient oxygen is provided to the sensor, the sensor is unable to accurately measure glucose concentration. Thus, depressed sensor function or inaccuracy is believed to be a result of problems in availability of oxygen to the enzyme and/or electrochemically active surface(s).

Accordingly, in an alternative example, an oxygen conduit (for example, a high oxygen solubility domain formed from silicone or fluorochemicals) is provided that extends from the ex vivo portion of the sensor to the in vivo portion of the sensor to increase oxygen availability to the enzyme. The oxygen conduit can be formed as a part of the coating (insulating) material or can be a separate conduit associated with the assembly of wires that forms the sensor.

FIG. 2B is a cross-sectional view through the sensor of FIG. 2A on line B-B, showing a core 39 having an exposed electrochemically active surface of at least a working electrode 38 surrounded by a sensing membrane 32. The core 39 is configured for multi-axis bending and can be stainless steel, titanium, tantalum, or a polymer. In general, the sensing membranes of the present disclosure include a plurality of domains or layers, for example, an interference domain 44, an enzyme domain 46, and a resistance domain 48, and may include additional domains, such as an electrode domain, a cell impermeable domain (not shown), an oxygen domain (not shown), a bioactive releasing membrane 70, and/or a biointerface membrane 68 (not shown), such as described in more detail below and/or in the above-cited U.S. patent publications. However, it is understood that a sensing membrane modified for other sensors, for example, by including fewer or additional domains is within the scope of the present disclosure.

### Membrane Systems

In some examples, one or more domains of the sensing membranes are formed from materials such as silicone, polytetrafluoroethylene, polyethylene-co-tetrafluoroethylene, polyolefin, polyester, polyalkylester, polyalkylcarbonate, polycarbonate, biostable polytetrafluoroethylene, homopolymers, copolymers, terpolymers of polyurethanes or polyurethane urea copolymer, polypropylene (PP), polyvinylchloride (PVC), polyvinylidene fluoride (PVDF), polybutylene terephthalate (PBT), polymethylmethacrylate (PMMA), polyethylene vinyl acetate, polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers. U.S. Pat. Pub. No. 2005/0245799, which is incorporated herein by reference in its entirety, describes biointerface and sensing membrane configurations and materials that may be applied to the presently disclosed sensor.

The sensing membrane can be deposited on the electrochemically active surfaces of the electrode material using known thin or thick film techniques (for example, spraying, electro-depositing, dipping, or the like). It is noted that the sensing membrane that surrounds the working electrode does not have to be the same structure as the sensing membrane that surrounds a reference electrode, etc. For example, the enzyme domain deposited over the working electrode does not necessarily need to be deposited over the reference and/or counter electrodes.

In the illustrated example, the sensor is an enzyme-based electrochemical sensor, wherein the working electrode 38 measures electronic current, e.g. detection of glucose utilizing glucose oxidase produces hydrogen peroxide as a by-product, H₂O₂ reacts with the surface of the working electrode producing two protons (2H+), two electrons (2e−) and one molecule of oxygen (O2) which produces the electronic current being detected, or via direct electron transfer of a redox system, e.g., a “wired enzyme” system, such as described in more detail above and as is appreciated by one skilled in the art. One or more potentiostats is employed to monitor the electrochemical reaction at the electrochemically active surface of the working electrode(s). The potentiostat applies a constant potential to the working electrode and its associated reference electrode to determine the current produced at the working electrode. The current that is produced at the working electrode (and flows through the circuitry to the counter electrode) is substantially proportional to the amount of H₂O₂ that diffuses to the working electrode or analyte that facilitates electron transfer in the wired enzyme system. The output signal is typically a raw data stream that is used to provide a useful value of the measured analyte concentration in a host to the host or doctor, for example.

Some alternative analyte sensors that can benefit from the systems and methods of the present disclosure include U.S. Pat. No. 5,711,861, to Ward et al., U.S. Pat. No. 6,642,015, to Vachon et al., U.S. Pat. No. 6,654,625, to Say et al., U.S. Pat. No. 6,565,509, to Say et al., U.S. Pat. No. 6,514,718, to Heller, U.S. Pat. No. 6,465,066, to Essenpreis et al., U.S. Pat. No. 6,214,185, to Offenbacher et al., U.S. Pat. No. 5,310,469, to Cunningham et al., and U.S. Pat. No. 5,683,562, to Shaffer et al., U.S. Pat. No. 6,579,690, to Bonnecaze et al., U.S. Pat. No. 6,484,046, to Say et al., U.S. Pat. No. 6,512,939, to Colvin et al., U.S. Pat. No. 6,424,847, to Mastrototaro et al., U.S. Pat. No. 6,424,847, to Mastrototaro et al., for example. All of the above patents are incorporated in their entirety herein by reference and are not inclusive of all applicable analyte sensors; in general, it should be understood that the disclosed examples are applicable to a variety of analyte sensor configurations. Exemplary Sensor Configurations

FIG. 2C is a cross-sectional view through the sensor of FIG. 2A on line B-B, showing a non-exposed electrochemically active surface of at least a working electrode 38 surrounded by a sensing membrane including a plurality of domains or layers, for example, the interference domain 44, the enzyme domain 46, and the resistance domain 48, and includes additional domains/membranes, such as an electrode domain, a cell impermeable domain (not shown), an oxygen domain (not shown), a bioactive releasing membrane 70, and/or a biointerface membrane 68 (not shown), such as described in more detail below. The bioactive releasing membrane 70 is positioned adjacent to working electrode 38 surface and does not cover working electrode 38 or the plurality of domains or layers, for example, the interference domain 44, the enzyme domain 46, and the resistance domain 48, of the sensing membrane 32 adjacent the working electrode surface(s). In one example, the bioactive releasing membrane 70 is positioned at the distal end 37 of sensor 34. In another example, the bioactive releasing membrane 70 straddles the electrochemically active portion of the working electrode 38, and does not cover the sensing membrane 32 associated with the working electrode 38.

FIG. 2D is a cross-sectional view through the sensor of FIG. 2A on line D-D of an exemplary bioactive releasing membrane deposition of sensor 34, where bioactive releasing membrane 70 is more distant from electrode 38 than resistance domain 48 and/or biointerface domain 68 and adjacent to, but not covering, the enzyme domain 46 or transducing element(s) and/or the interference domain 44, and/or sensing region or the electrochemically active surface of the sensing region. Bioactive releasing membrane 70 can be arranged on sensor 34 as shown in FIG. 2D using one or more of screen printing, spray coating, or dip coating methods.

FIG. 2E is a cross-sectional view through the sensor of FIG. 2A on line B-B of another exemplary bioactive releasing membrane deposition where bioactive releasing membrane 70 is more distant from electrode 38 than resistance domain 48 and/or biointerface layer 68 and adjacent to, and is generally covering only the tip or distal end 37 of sensor 34, up to and adjacent to, while not covering, enzyme domain 46 or transducing element(s) and/or the interference domain 44, and/or sensing region or the electrochemically active surface of the sensing region. Bioactive releasing membrane 70 can be arranged on sensor 34 as shown in FIG. 2E using one or more of screen printing, spray coating, or dip coating methods.

FIG. 2F can be considered to build on a general structure as depicted in FIG. 2A, in that two or more additional layers are added to create one or more additional electrodes. Methods for selectively removing two or more windows to create two or more electrodes can also be employed. For example, by adding another conductive layer 38b and insulating layer 35b under a reference electrode layer 30, then two electrodes (first and (optional) second working electrodes, etc.) can be formed, yielding a dual electrode sensor or multielectrode sensor. The same concept can be applied to create, a counter electrode, electrodes to measure additional analytes (e.g., oxygen), and the like, for example. FIG. 2G illustrates a sensor having an additional electrode 38b, wherein the windows are selectively removed to expose working electrodes 38a, 38b in between a reference electrode (including multiple segments) 30, with a small amount of insulator 35a, 35b exposed therebetween.

While some figures herein illustrate sensors that may have a coaxial core and a circular or elliptical cross-section, in other examples of sensor systems including bioactive releasing membrane (s), the sensor may be a substantially planar sensor, as shown in the cross-section for illustration purposes in FIG. 2H. For example, as shown in FIG. 2H, the continuous analyte sensing device 100 can include a substantially planar substrate 142, as well as an interference domain 144, an enzyme domain 146, a resistance domain 148, and a biointerface/bioprotective domain 168 and/or a bioactive releasing domain 170 arranged in a substantially planar fashion around the substantially planar substrate 142 with one or more working electrodes. Referring to FIGS. 2G-2H, in some examples, the reference electrode 30 comprises a silver-containing material applied over at least a portion of the insulating material 35. In some examples, the silver-containing material is applied using thin film and/or thick film techniques, such as but not limited to dipping, spraying, printing, electro-depositing, vapor deposition, spin coating, and sputter deposition, as described elsewhere herein. For example, a silver or silver-chloride-containing paint (or similar formulation) is applied to a reel of the insulated conductive core, in examples. In another example, the reel of insulated elongated body (or core) is cut into single unit pieces (e.g., “singularized”) and a silver-containing ink is pad printed thereon. In still other examples, the silver-containing material is applied as a silver foil. For example, an adhesive can be applied to an insulated elongated body, around which the silver foil is then wrapped in. Alternatively, the sensor can be rolled in Ag/AgCl particles, such that a sufficient amount of silver sticks to and/or embeds into and/or otherwise adheres to the adhesive for the particles to function as the reference electrode. In some examples, the sensor's reference electrode includes a sufficient amount of chloridized silver that the sensor measures and/or detects the analyte for at least three days.

In some examples, the sensor is formed from an elongated body 33 (e.g., elongated conductive body), such as that shown in FIG. 2G, wherein the elongated body includes a core 39, a first layer 38a, an insulator 35a, and a layer of silver-containing material 30. In some examples, such as that shown in FIG. 2H, the electrochemically active surface of the elongated body (e.g., also the (electroactive) surface of the first layer 38a) is exposed by formation of a window 31 through both the silver-containing material and the insulator. In one exemplary example, the elongated body of FIG. 2G is provided as an extended length on a reel that is singularized into a plurality of pieces having a length (e.g., less than 0.5, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5 or 24-inch or longer lengths) and suitable for a selected sensor configuration. For example, a first sensor configured for transcutaneous implantation can employ 2.5-inch lengths, while a second sensor configured for transcutaneous implantation can employ 3-inch lengths. In another example, a first sensor configured for implantation into a peripheral vein of an adult host can employ a 3-inch length, while a second sensor configured for implantation into a central vein of an adult host can employ a 12-inch length. The window is formed on each sensor, such as by scraping and or etching a radial window through the silver-containing material and the insulator such that the platinum surface is exposed (e.g., the electrochemically active surface of the “working electrode”). In some examples, a reel of elongated body is singularized and then the windows are formed. In other examples, the windows are formed along the length of the reel of elongated body, and then later singularized. In a further example, additional manufacturing steps are performed prior to singularization. A sensing membrane 32 is applied to the exposed electrochemically active surface (e.g., the working electrode) defined by the edges of the window, such that the electrochemically active surface can function as the working electrode of the sensor to generate a signal associated with an analyte (e.g., when the sensor is in contact with a sample of a host). Alternative manufacturing techniques and/or sequences of steps can be used to produce sensors having the configuration shown in FIG. 2H, such as but not limited to masking a portion of the elongated body (or core) prior to application of the insulator and the silver-containing material.

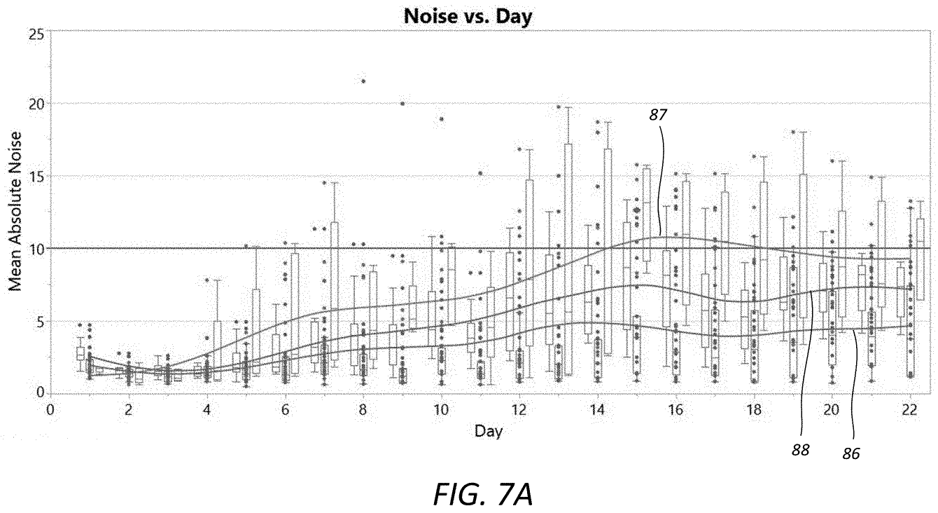

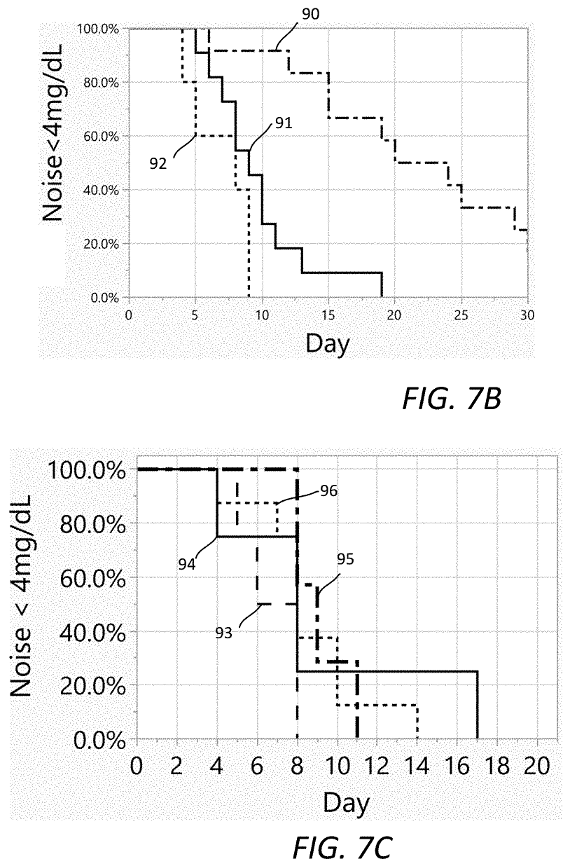

FIG. 2G is an illustration showing layers cut away, but in the fabrication process the material typically obtained has all layers ending at a tip. A step of removing layers 30 and 30 can be performed so as to form window(s). FIG. 2I illustrates the results of this removal/cutting away process through a side-view/cross-section. The removal process can be accomplished by the methods already described or other methods as known in the art. In one example the removal step is conducted, e.g., by laser skiving, and can be performed in a reel-to-reel process on a continuous strand. The removed area can be stepped, for example, by removing different layers by different lengths (FIG. 2I). In such a fabrication method involving a continuous strand, the sensors can be singularized after the removal step creating a singulation 29 (FIGS. 7A-7C). In some examples, if the core is a metal, an end cap may be employed, e.g., by dipping, spraying, shrink tubing, crimp wrapping, etc., an insulating or other isolating material onto the tip. If the core is a polymer (e.g., hydrophobic material), an end cap may not be necessary. For example, in the sensor depicted in FIG. 2I, an end cap 40 (e.g., of a polymer or an insulating material) or other structure may be provided over the core (e.g., if the core 39 is not insulating).

FIG. 2J can be considered to build on a general structure as depicted in FIG. 2G, in that two or more additional layers are added to create one or more additional electrodes. Methods for selectively removing two or more windows to create two or more electrodes can also be employed. For example, by adding another conductive layer 38b and insulating layer 35b under a reference electrode layer 30, then two electrodes (first and second working electrodes) can be formed, yielding a dual electrode sensor. The same concept can be applied to create, a counter electrode, electrodes to measure additional analytes (e.g., oxygen), and the like, for example.

FIG. 2K illustrates a sensor having an additional electrode 38b (as compared to FIGS. 2G-21), wherein the windows are selectively removed to expose working electrodes 38a, 38b in between a reference electrode (including multiple segments) 30, with a small amount of insulator 35a, 35b exposed therebetween. FIG. 2L illustrates another example, wherein selective removal of the various layers is stepped to expose the electrodes 38a, 38b and insulators 35a, 35b along the length of the elongated body.

FIG. 2J is a cross-sectional view through an alternative sensor configuration, showing a non-exposed electrochemically active surface of at least a working electrode 38 surrounded by a sensing membrane 32 including a plurality of domains or layers, for example, the interference domain 44, the enzyme domain 46, and the resistance domain 48, and includes additional domains/membranes, such as an electrode domain, a cell impermeable domain (not shown), an oxygen domain (not shown), a bioactive releasing membrane 70, and/or a biointerface membrane 68 (not shown), such as described in more detail below. The bioactive releasing membrane 70 is positioned adjacent to working electrode 38 surface and does not cover working electrode 38 or the plurality of domains or layers, for example, the interference domain 44, the enzyme domain 46, and the resistance domain 48, of the sensing membrane 32 associated with the working electrode(s). As shown in FIG. 2J bioactive diffusion adjustment membrane 73 is provided, adjacent bioactive releasing membrane 70. In one example, the diffusion adjustment membrane 73 is directly adjacent the bioactive releasing membrane 70. In another example, the diffusion adjustment membrane 73 is chemically, structurally or functionally different from the bioactive releasing membrane 70. In another example, the diffusion adjustment membrane 73 is a block copolymer, e.g., a polyurethane block polymer with a hard segment and a soft segment, where the soft segment can comprise a hydrophobic portion, a hydrophilic portion, or a combination of hydrophobic/hydrophilic portion. Each of the of hydrophobic/hydrophilic portions can independently be of a different average molecular weight or chain length. In another example, the diffusion adjustment membrane 73 is a segmented block copolymer of soft segment comprising combinations of hydrophobic/hydrophilic portions, such as polyols (polyethylene oxides, polyethylenepropylene oxides, poly tetrahydrofuran or polytetramethylene oxide, polyethers, polysiloxanes, polyamines, polysiloxane amine, polyester, polyalkylester, polyalkylcarbonate, polycarbonate and one or more independent hard segments, e.g. an aliphatic or aromatic diisocyanate such as norbornane diisocyanate (NBDI), isophorone diisocynate (IPDI), tolylene diisocynate (TDI), 1,3-phenylene diisocyanate (MPDI), trans-1,3-bis(isocynatomethyl) cyclohexane (1,3-H6XDI), bicyclohexylmethane-4,4′-diisocynate(HMDI), 4,4′-Diphenylmethane diisocynate (MDI), trans-1,4-bis(isocynatomethyl) cyclohexane (1,4-H6XDI), 1,4-cyclohexyl diisocynate (CHDI), 1,4-phenylene diisocynate (PPDI), 3,3′-Dimethyl-4,4′-biphenyldiisocyanate (TODI), 1,6-hexamethylene diisocyanate (HDI), or combinations thereof.

In another example, the diffusion adjustment membrane 73 is a multi-block copolymer. In another example, the diffusion adjustment membrane is annealed to provide stable separated phases and/or diffusion channels for release of bioactive agent. In examples, the diffusion adjustment membrane 73 is continuously, semi-continuously, or segmentally (randomly or in a pattern) applied over the bioactive releasing membrane 70.

In some examples, the silver-containing material is applied to the sensor (e.g., the insulated conductive core) in a substantially continuous process, such as described elsewhere herein. Accordingly, in some examples, the silver-containing material is applied in a fully-automated process. In other examples, the silver-containing material is applied in a semi-automated process.

While the methods of the present disclosure are especially well suited for use with small structured-, micro- or small diameter sensors, the methods can also be suitable for use with larger diameter sensors, e.g., sensors of 1 mm to about 2 mm or more in diameter.

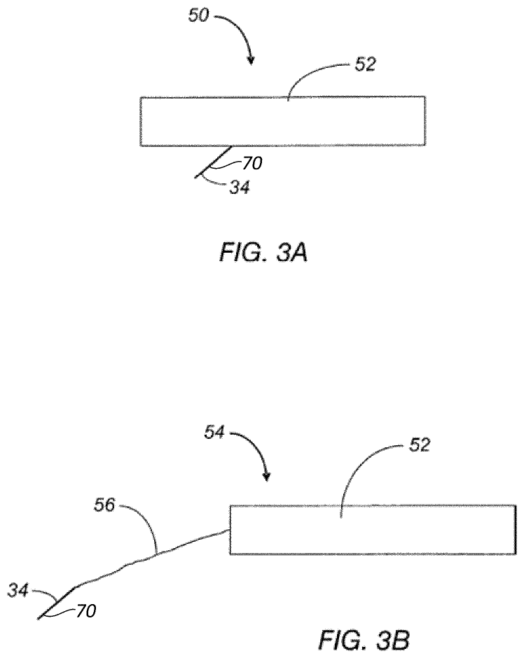

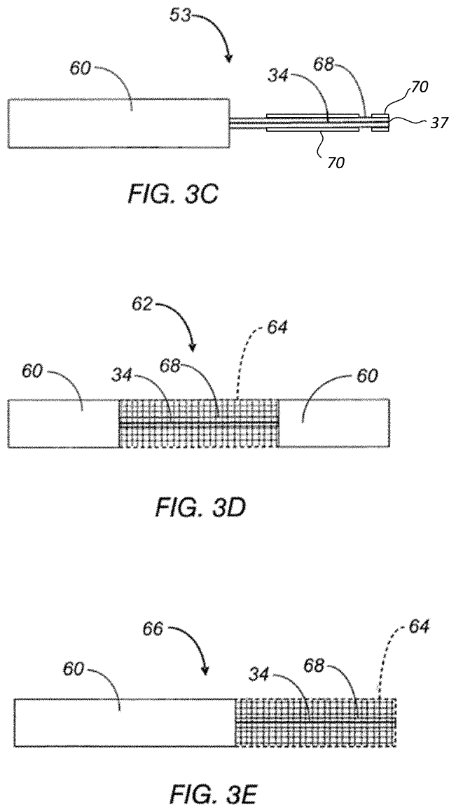

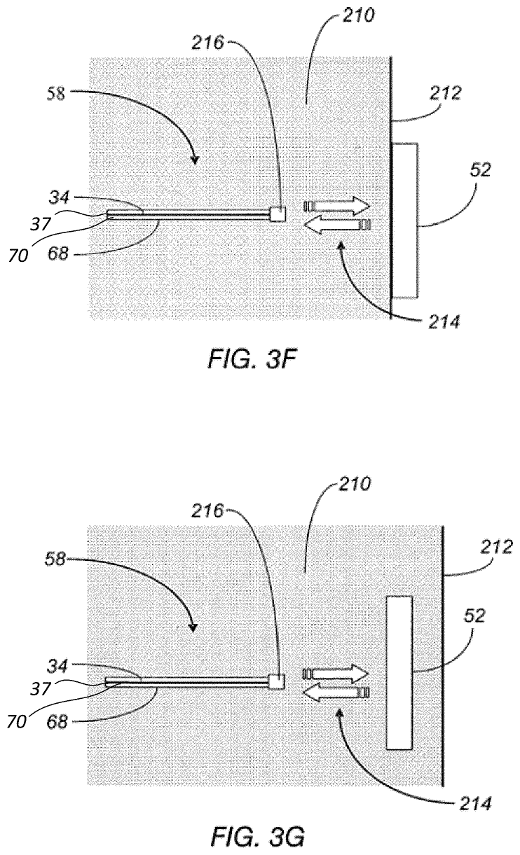

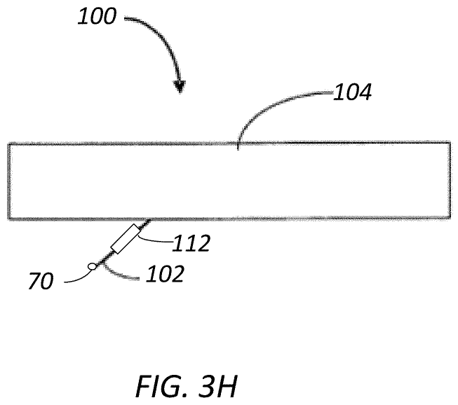

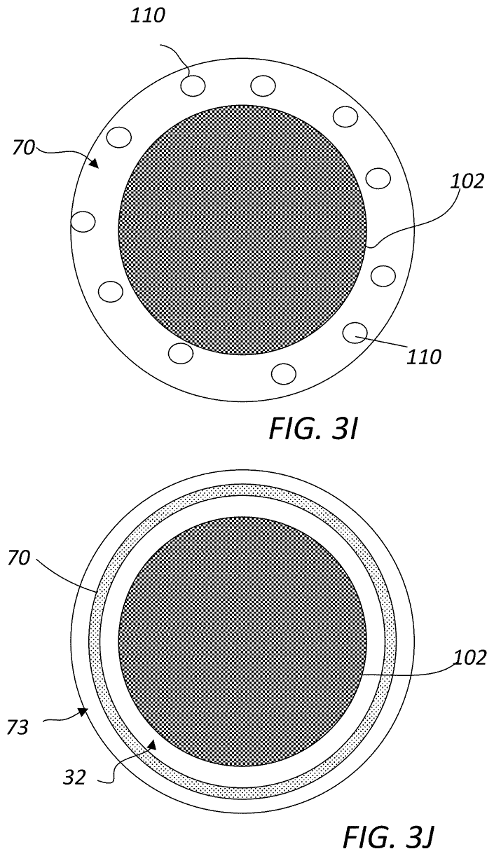

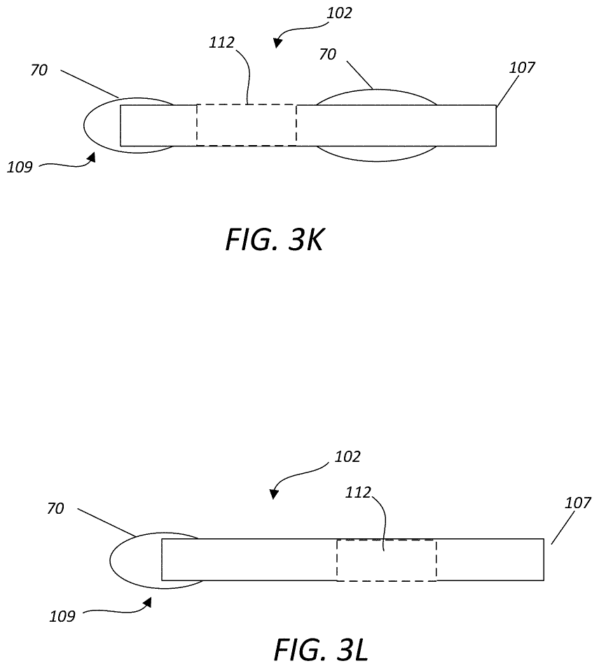

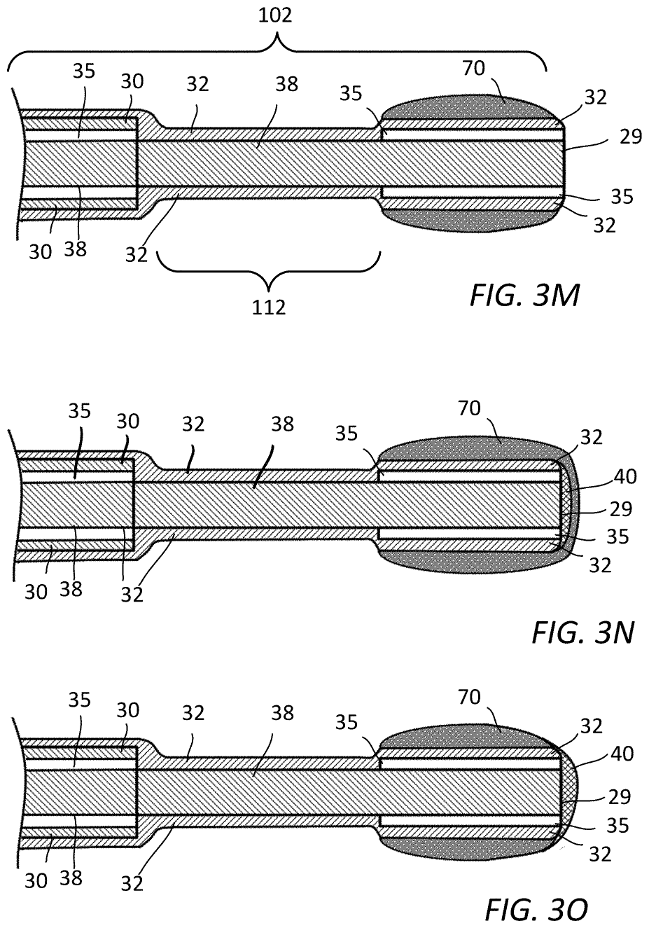

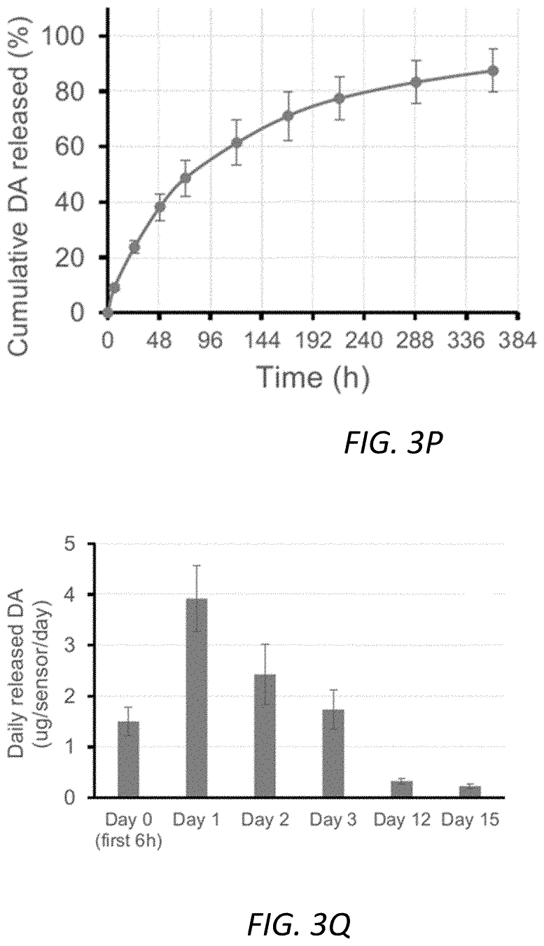

FIG. 3A is a side schematic view of a transcutaneous analyte sensor 50 in one example. The sensor 50 includes a mounting unit 52 adapted for mounting on the skin of a host, a small (diameter) structure sensor 34 (as defined herein) adapted for transdermal insertion through the skin of a host, and an electrical connection configured to provide secure electrical contact between the sensor and the electronics preferably housed within the mounting unit 52. In general, the mounting unit 52 is designed to maintain the integrity of the sensor in the host so as to reduce or eliminate translation of motion between the mounting unit, the host, and/or the sensor. See U.S. Pat. Pub. No. 2006/0020187, filed on Mar. 10, 2005 and entitled, “TRANSCUTANEOUS ANALYTE SENSOR,” which is incorporated herein by reference in its entirety. In one example, a bioactive releasing membrane is formed onto the sensing mechanism 36 as described in more detail below.

FIG. 3B is a side schematic view of a transcutaneous analyte sensor 54 in an alternative example. The transcutaneous analyte sensor 54 includes a mounting unit 52 wherein the sensing mechanism 36 comprises a small structure as defined herein and is tethered to the mounting unit 52 via a cable 56 (alternatively, a wireless connection can be utilized). The mounting unit is adapted for mounting on the skin of a host and is operably connected via a tether, or the like, to a small structured sensor 34 adapted for transdermal insertion through the skin of a host and measurement of the analyte therein; see, for example, U.S. Pat. No. 6,558,330, to Causey III et al., which is incorporated herein by reference in its entirety. In one example, a bioactive releasing membrane 70 is formed onto at least a part of the sensing mechanism 36 as described in more detail below.

The sensor of the present disclosure may be inserted into a variety of locations on the host's body, such as the abdomen, the thigh, the upper arm, and the neck or behind the ear. Although the present disclosure may suggest insertion through the abdominal region, the systems and methods described herein are limited neither to the abdominal nor to the subcutaneous insertions. One skilled in the art appreciates that these systems and methods may be implemented and/or modified for other insertion sites and may be dependent upon the type, configuration, and dimensions of the analyte sensor.

Transcutaneous continuous analyte sensors can be used in vivo over various lengths of time. For example, the device includes a sensor, for measuring the analyte in the host, a porous, biocompatible matrix covering at least a portion of the sensor, and an applicator, for inserting the sensor through the host's skin. In some examples, the sensor has architecture with at least one dimension less than about 1 mm. Examples of such a structure are shown in FIGS. 3A and 3B, as described elsewhere herein. However, one skilled in the art will recognize that alternative configurations are possible and may be desirable, depending upon factors such as intended location of insertion, for example. The sensor is inserted through the host's skin and into the underlying tissue, such as soft tissue or fatty tissue.

After insertion, fluid moves into the spacer, e.g., a biocompatible matrix or membrane, such as the bioactive releasing membrane 70 and/or biointerface membrane 68, creating a fluid-filled pocket therein. This process may occur immediately or may take place over a period of time, such as several minutes or hours post insertion. A signal from the sensor is then detected, such as by the sensor electronics unit located in the mounting unit on the surface of the host's skin. In general, the sensor may be used continuously for a period of days, such as 1 to 7 days, 14 days, or 21 days. After use, the sensor is simply removed from the host's skin. In one example, the host may repeat the insertion and detection steps as many times as desired. In some implementations, the sensor may be removed after about 3 days, and then another sensor inserted, and so on. Similarly, in other implementations, the sensor is removed after about 3, 5, 7, 10 or 14 days, followed by insertion of a new sensor, and so on.

Some examples of transcutaneous analyte sensors are described in U.S. Pat. No. 8,133,178, to Brauker et al., which is incorporated herein by reference in its entirety, as well as U.S. Pat. No. 8,828,201, Simpson, et al.; U.S. Pat. No. 9,131,885, Simpson, et al.; U.S. Pat. No. 9,237,864, Simpson, et al.; and 9,763,608, Simpson, et al., each of which is incorporated by reference in its entirety herein. In general, transcutaneous analyte sensors comprise the sensor and a mounting unit with electronics associated therewith.

In general, the mounting unit includes a base adapted for mounting on the skin of a host, a sensor adapted for transdermal insertion through the skin of a host, and one or more contacts configured to provide secure electrical contact between the sensor and the sensor electronics. The mounting unit is designed to maintain the integrity of the sensor in the host so as to reduce or eliminate translation of motion between the mounting unit, the host, and/or the sensor. The base can be formed from a variety of hard or soft materials, and preferably comprises a low profile for minimizing protrusion of the device from the host during use. In some examples, the base is formed at least partially from a flexible material, which is believed to provide numerous advantages over conventional transcutaneous sensors, which, unfortunately, can suffer from motion-related artifacts associated with the host's movement when the host is using the device. For example, when a transcutaneous analyte sensor is inserted into the host, various movements of the sensor (for example, relative movement between the in vivo portion and the ex vivo portion, movement of the skin, and/or movement within the host (dermis or subcutaneous)) create stresses on the device and can produce noise in the sensor signal. It is believed that even small movements of the skin can translate to discomfort and/or motion-related artifact, which can be reduced or obviated by a flexible or articulated base. Thus, by providing flexibility and/or articulation of the device against the host's skin, better conformity of the sensor system to the regular use and movements of the host can be achieved. Flexibility or articulation is believed to increase adhesion (with the use of an adhesive pad) of the mounting unit onto the skin, thereby decreasing motion-related artifact that can otherwise translate from the host's movements and reduced sensor performance.

In certain examples, the mounting unit is provided with an adhesive pad, preferably disposed on the mounting unit's back surface and preferably including a releasable backing layer. Thus, removing the backing layer and pressing the base portion of the mounting unit onto the host's skin adheres the mounting unit to the host's skin. Additionally or alternatively, an adhesive pad can be placed over some or all of the sensor system after sensor insertion is complete to ensure adhesion, and optionally to ensure an airtight seal or watertight seal around the wound exit-site (or sensor insertion site). Appropriate adhesive pads can be chosen and designed to stretch, elongate, conform to, and/or aerate the region (e.g., host's skin).

In examples, the adhesive pad is formed from spun-laced, open- or closed-cell foam, and/or non-woven fibers, and includes an adhesive disposed thereon, however a variety of adhesive pads appropriate for adhesion to the host's skin can be used, as is appreciated by one skilled in the art of medical adhesive pads. In some examples, a double-sided adhesive pad is used to adhere the mounting unit to the host's skin. In other examples, the adhesive pad includes a foam layer, for example, a layer wherein the foam is disposed between the adhesive pad's side edges and acts as a shock absorber.

In some examples, the surface area of the adhesive pad is greater than the surface area of the mounting unit's back surface. Alternatively, the adhesive pad can be sized with substantially the same surface area as the back surface of the base portion. Preferably, the adhesive pad has a surface area on the side to be mounted on the host's skin that is greater than about 1, 1.25, 1.5, 1.75, 2, 2.25, or 2.5, times the surface area of the back surface of the mounting unit base. Such a greater surface area can increase adhesion between the mounting unit and the host's skin, minimize movement between the mounting unit and the host's skin, and/or protect the wound exit-site (sensor insertion site) from environmental and/or biological contamination. In some alternative examples, however, the adhesive pad can be smaller in surface area than the back surface assuming a sufficient adhesion can be accomplished.

In some examples, the adhesive pad is substantially the same shape as the back surface of the base, although other shapes can also be advantageously employed, for example, butterfly-shaped, round, square, or rectangular. The adhesive pad backing can be designed for two-step release, for example, a primary release wherein only a portion of the adhesive pad is initially exposed to allow adjustable positioning of the device, and a secondary release wherein the remaining adhesive pad is later exposed to firmly and securely adhere the device to the host's skin once appropriately positioned. The adhesive pad is preferably waterproof. Preferably, a stretch-release adhesive pad is provided on the back surface of the base portion to enable easy release from the host's skin at the end of the useable life of the sensor.

In some circumstances, it has been found that a conventional bond between the adhesive pad and the mounting unit may not be sufficient, for example, due to humidity that can cause release of the adhesive pad from the mounting unit. Accordingly, in some examples, the adhesive pad can be bonded using a bonding agent activated by or accelerated by an ultraviolet, acoustic, radio frequency, or humidity cure. In some examples, a eutectic bond of first and second composite materials can form a strong adhesion. In some examples, the surface of the mounting unit can be pretreated utilizing ozone, plasma, chemicals, or the like, in order to enhance the bondability of the surface.

A bioactive agent is preferably applied locally at the insertion site prior to or during sensor insertion. Suitable bioactive agents include those which are known to discourage or prevent bacterial growth and infection, for example, anti-inflammatory agents, antimicrobials, antibiotics, or the like. It is believed that the diffusion or presence of a bioactive agent can aid in prevention or elimination of bacteria adjacent to the exit-site. Additionally or alternatively, the bioactive agent can be integral with or coated on the adhesive pad, or no bioactive agent at all is employed.

In some examples, an applicator is provided for inserting the sensor through the host's skin at the appropriate insertion angle with the aid of a needle, and for subsequent removal of the needle using a continuous push-pull action. Preferably, the applicator comprises an applicator body that guides the applicator and includes an applicator body base configured to mate with the mounting unit during insertion of the sensor into the host. The mate between the applicator body base and the mounting unit can use any known mating configuration, for example, a snap-fit, a press-fit, an interference-fit, or the like, to discourage separation during use. One or more release latches enable release of the applicator body base, for example, when the applicator body base is snap fit into the mounting unit.

The sensor electronics includes hardware, firmware, and/or software that enable measurement of levels of the analyte via the sensor. For example, the sensor electronics can comprise a potentiostat, a power source for providing power to the sensor, other components useful for signal processing, and preferably an RF module for transmitting data from the sensor electronics to a receiver. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, or a processor. Preferably, sensor electronics comprise systems and methods for processing sensor analyte data. Examples of systems and methods for processing sensor analyte data are described in more detail below and in U.S. Pat. No. 7,778,680, filed Aug. 1, 2003, and entitled, “SYSTEM AND METHODS FOR PROCESSING ANALYTE SENSOR DATA.”

In this example, after insertion of the sensor using the applicator, and subsequent release of the applicator from the mounting unit, the sensor electronics are configured to releasably mate with the mounting unit. In examples, the electronics are configured with programming, for example initialization, calibration reset, failure testing, or the like, each time it is initially inserted into the mounting unit and/or each time it initially communicates with the sensor.

### Sensor Electronics

The following description of electronics associated with the sensor is applicable to a variety of continuous analyte sensors, such as non-invasive, minimally invasive, and/or invasive (e.g., transcutaneous and wholly implantable) sensors. For example, the sensor electronics and data processing as well as the receiver electronics and data processing described below can be incorporated into the wholly implantable glucose sensor disclosed in U.S. Pat. Pub. No. 2005/0245799, filed May 3, 2004 and entitled “IMPLANTABLE ANALYTE SENSOR” and U.S. Pat. Pub. No. 2006/0015020, filed Jul. 6, 2004 and entitled, “SYSTEMS AND METHODS FOR MANUFACTURE OF AN ANALYTE-MEASURING DEVICE INCLUDING A MEMBRANE SYSTEM”.

In one example, a potentiostat, which is operably connected to an electrode system (such as described above) provides a voltage to the electrodes, which biases the sensor to enable measurement of a current signal indicative of the analyte concentration in the host (also referred to as the analog portion). In some examples, the potentiostat includes a resistor that translates the current into voltage. In some alternative examples, a current to frequency converter is provided that is configured to continuously integrate the measured current, for example, using a charge counting device. An A/D converter digitizes the analog signal into a digital signal, also referred to as “counts” for processing. Accordingly, the resulting raw data stream in counts, also referred to as raw sensor data, is directly related to the current measured by the potentiostat.

A processor module includes the central control unit that controls the processing of the sensor electronics. In some examples, the processor module includes a microprocessor, however a computer system other than a microprocessor can be used to process data as described herein, for example an ASIC can be used for some or all of the sensor's central processing. The processor typically provides semi-permanent storage of data, for example, storing data such as sensor identifier (ID) and programming to process data streams (for example, programming for data smoothing and/or replacement of signal artifacts such as is described in U.S. Pat. No. 8,010,174, filed Aug. 22, 2003, and entitled, “SYSTEMS AND METHODS FOR REPLACING SIGNAL ARTIFACTS IN A GLUCOSE SENSOR DATA STREAM”). The processor additionally can be used for the system's cache memory, for example for temporarily storing recent sensor data. In some examples, the processor module comprises memory storage components such as ROM, RAM, dynamic-RAM, static-RAM, non-static RAM, EEPROM, rewritable ROMs, flash memory, or the like.

In some examples, the processor module comprises a digital filter, for example, an IIR or FIR filter, configured to smooth the raw data stream from the A/D converter. Generally, digital filters are programmed to filter data sampled at a predetermined time interval (also referred to as a sample rate). In some examples, wherein the potentiostat is configured to measure the analyte at discrete time intervals, these time intervals determine the sample rate of the digital filter. In some alternative examples, wherein the potentiostat is configured to continuously measure the analyte, for example, using a current-to-frequency converter as described above, the processor module can be programmed to request a digital value from the A/D converter at a predetermined time interval, also referred to as the acquisition time. In these alternative examples, the values obtained by the processor are advantageously averaged over the acquisition time due the continuity of the current measurement. Accordingly, the acquisition time determines the sample rate of the digital filter. In one example, the processor module is configured with a programmable acquisition time, namely, the predetermined time interval for requesting the digital value from the A/D converter is programmable by a user within the digital circuitry of the processor module. An acquisition time of from about 2 seconds to about 512 seconds is preferred; however any acquisition time can be programmed into the processor module. A programmable acquisition time is advantageous in optimizing noise filtration, time lag, and processing/battery power.

Preferably, the processor module is configured to build the data packet for transmission to an outside source, for example, an RF transmission to a receiver as described in more detail below. Generally, the data packet comprises a plurality of bits that can include a sensor ID code, raw data, filtered data, and/or error detection or correction. The processor module can be configured to transmit any combination of raw and/or filtered data.

In some examples, the processor module further comprises a transmitter portion that determines the transmission interval of the sensor data to a receiver, or the like. In some examples, the transmitter portion, which determines the interval of transmission, is configured to be programmable. In one such example, a coefficient can be chosen (e.g., a number of from about 1 to about 100, or more), wherein the coefficient is multiplied by the acquisition time (or sampling rate), such as described above, to define the transmission interval of the data packet. Thus, in some examples, the transmission interval is programmable between about 2 seconds and about 850 minutes, more preferably between about 30 second and 5 minutes; however, any transmission interval can be programmable or programmed into the processor module. However, a variety of alternative systems and methods for providing a programmable transmission interval can also be employed. By providing a programmable transmission interval, data transmission can be customized to meet a variety of design criteria (e.g., reduced battery consumption, timeliness of reporting sensor values, etc.)

Conventional glucose sensors measure current in the nanoampere range. In contrast to conventional glucose sensors, the presently disclosed sensors are configured to measure the current flow in the picoampere range, and in some examples, femtoamps. Namely, for every unit (mg/dL) of glucose measured, at least one picoampere of current is measured. Preferably, the analog portion of the A/D converter is configured to continuously measure the current flowing at the working electrode and to convert the current measurement to digital values representative of the current. In examples, the current flow is measured by a charge counting device (e.g., a capacitor). Thus, a signal is provided, whereby a high sensitivity maximizes the signal received by a minimal amount of measured hydrogen peroxide (e.g., minimal glucose requirements without sacrificing accuracy even in low glucose ranges), reducing the sensitivity to oxygen limitations in vivo (e.g., in oxygen-dependent glucose sensors).

A battery is operably connected to the sensor electronics and provides the power for the sensor. In examples, the battery is a lithium manganese dioxide battery; however, any appropriately sized and powered battery can be used (for example, AAA, nickel-cadmium, zinc-carbon, alkaline, lithium, nickel-metal hydride, lithium-ion, zinc-air, zinc-mercury oxide, silver-zinc, and/or hermetically-sealed). In some examples, the battery is rechargeable, and/or a plurality of batteries can be used to power the system. The sensor can be transcutaneously powered via an inductive coupling, for example. In some examples, a quartz crystal is operably connected to the processor and maintains system time for the computer system as a whole, for example for the programmable acquisition time within the processor module.

Optional temperature probe can be provided, wherein the temperature probe is located on the electronics assembly or the glucose sensor itself. The temperature probe can be used to measure ambient temperature in the vicinity of the glucose sensor. This temperature measurement can be used to add temperature compensation to the calculated glucose value.

An RF module is operably connected to the processor and transmits the sensor data from the sensor to a receiver within a wireless transmission via antenna. In some examples, a second quartz crystal provides the time base for the RF carrier frequency used for data transmissions from the RF transceiver. In some alternative examples, however, other mechanisms, such as optical, infrared radiation (IR), ultrasonic, or the like, can be used to transmit and/or receive data.

In the RF telemetry module of the present disclosure, the hardware and software are designed for low power requirements to increase the longevity of the device (for example, to enable a life of from about 3 to about 24 months, or more) with maximum RF transmittance from the in vivo environment to the ex vivo environment for wholly implantable sensors (for example, a distance of from about one to ten meters or more). Preferably, a high frequency carrier signal of from about 402 MHz to about 433 MHz is employed in order to maintain lower power requirements. Additionally, in wholly implantable devices, the carrier frequency is adapted for physiological attenuation levels, which is accomplished by tuning the RF module in a simulated in vivo environment to ensure RF functionality after implantation; accordingly, the preferred glucose sensor can sustain sensor function for 3 months, 6 months, 12 months, or 24 months or more.

In some examples, output signal (from the sensor electronics) is sent to a receiver (e.g., a computer or other communication station). The output signal is typically a raw data stream that is used to provide a useful value of the measured analyte concentration to a patient or a doctor, for example. In some examples, the raw data stream can be continuously or periodically algorithmically smoothed or otherwise modified to diminish outlying points that do not accurately represent the analyte concentration, for example due to signal noise or other signal artifacts, such as described in U.S. Pat. No. 6,931,327, entitled, “SYSTEMS AND METHODS FOR REPLACING SIGNAL ARTIFACTS IN A GLUCOSE SENSOR DATA STREAM,” filed Aug. 1, 2003, which is incorporated herein by reference in its entirety.

When a sensor is first implanted into host tissue, the sensor and receiver are initialized. This can be referred to as start-up mode, and involves optionally resetting the sensor data and calibrating the sensor. In selected examples, mating the electronics unit to the mounting unit triggers a start-up mode. In other examples, the start-up mode is triggered by the receiver.

### Receiver

In some examples, the sensor electronics are wirelessly connected to a receiver via one- or two-way RF transmissions or the like. However, a wired connection is also contemplated. The receiver provides much of the processing and display of the sensor data, and can be selectively worn and/or removed at the host's convenience. Thus, the sensor system can be discreetly worn, and the receiver, which provides much of the processing and display of the sensor data, can be selectively worn and/or removed at the host's convenience. Particularly, the receiver includes programming for retrospectively and/or prospectively initiating a calibration, converting sensor data, updating the calibration, evaluating received reference and sensor data, and evaluating the calibration for the analyte sensor, such as described in more detail with reference to U.S. Pat. No. 7,778,680, filed Aug. 1, 2003 and entitled, “SYSTEM AND METHODS FOR PROCESSING ANALYTE SENSOR DATA.”

FIG. 3C is a side schematic view of a wholly implantable analyte sensor 53 in one example. The sensor includes a sensor body 60 suitable for subcutaneous implantation and includes a small structured sensor 34 as defined herein. Published U.S. Pat. Pub. No. 2004/0199059, to Brauker et al. describes systems and methods suitable for the sensor body 60, and is incorporated herein by reference in its entirety. In one example, a biointerface membrane 68 is formed onto the sensing mechanism 36 as described in more detail elsewhere herein. The sensor body 60 includes sensor electronics and preferably communicates with a receiver as described in more detail, above. As shown in FIG. 3C, bioactive releasing membrane 70 is disposed on at least a portion of biointerface membrane 68 and/or sensing membrane 32.

FIG. 3D is a side schematic view of a wholly implantable analyte sensor 62 in an alternative example. The wholly implantable analyte sensor 62 includes a sensor body 60 and a small structured sensor 34 as defined herein. The sensor body 60 includes sensor electronics and preferably communicates with a receiver as described in more detail, above.

In one example, a biointerface membrane 68 is formed onto the sensing mechanism 36 as described in more detail elsewhere herein. In another example, bioactive releasing membrane 70 is formed on at least a portion of the sensing mechanism 36. In another example, bioactive releasing membrane 70 is formed on discrete, separated portions of the sensing mechanism 36. In yet another example, the biointerface membrane 68 is formed onto at least a portion of the bioactive releasing membrane 70. In yet another example, the bioactive releasing membrane 70 is formed onto at least a portion of the biointerface membrane 68. In one example, a matrix or framework 64 surrounds the sensing mechanism 36 for protecting the sensor from some foreign body processes, for example, by causing tissue to compress against or around the framework 64 rather than the sensing mechanism 36.

In general, the optional protective framework 64 is formed from a two-dimensional or three-dimensional flexible, semi-rigid, or rigid matrix (e.g., mesh), and which includes spaces or pores through which the analyte can pass. In some examples, the framework is incorporated as a part of the biointerface membrane, however a separate framework can be provided. While not wishing to be bound by theory, it is believed that the framework 64 protects the small structured sensing mechanism from mechanical forces created in vivo.

FIG. 3E is a side schematic view of a wholly implantable analyte sensor 66 in another alternate example. The sensor 66 includes a sensor body 60 and a small structured sensor 34, as defined herein, with biointerface membrane 68 and/or bioactive releasing membrane 70 such as described in more detail elsewhere herein. Preferably, a framework 64 protects the sensing mechanism 36 such as described in more detail above. The sensor body 60 includes sensor electronics and preferably communicates with a receiver as described in more detail, above.

In certain examples, the sensing device, which is adapted to be wholly implanted into the host, such as in the soft tissue beneath the skin, is implanted subcutaneously, such as in the abdomen of the host, for example. One skilled in the art appreciates a variety of suitable implantation sites available due to the sensor's small size. In some examples, the sensor architecture is less than about 0.5 mm in at least one dimension, for example a wire-based sensor with a diameter of less than about 0.5 mm. In another exemplary example, for example, the sensor may be 0.5 mm thick, 3 mm in length and 2 cm in width, such as possibly a narrow substrate, needle, wire, rod, sheet, or pocket. In another exemplary example, a plurality of about 1 mm wide wires about 5 mm in length could be connected at their first ends, producing a forked sensor structure. In still another example, a 1 mm wide sensor could be coiled, to produce a planar, spiraled sensor structure. Although a few examples are cited above, numerous other useful examples are contemplated by the present disclosure, as is appreciated by one skilled in the art.

Post implantation, a period of time is allowed for tissue ingrowth within the biointerface. The length of time required for tissue ingrowth varies from host to host, such as about a week to about 3 weeks, although other time periods are also possible. Once a mature bed of vascularized tissue has grown into the biointerface, a signal can be detected from the sensor, as described elsewhere herein and in U.S. Pat. Pub. No. 2005/0245799, to Brauker et al., entitled IMPLANTABLE ANALYTE SENSOR, incorporated herein in its entirety. Long term sensors can remain implanted and produce glucose signal information from months to years, as described in the above-cited patent application.

In certain examples, the device is configured such that the sensing unit is separated from the electronics unit by a tether or cable, or a similar structure, similar to that illustrated in FIG. 3B. One skilled in the art will recognize that a variety of known and useful means may be used to tether the sensor to the electronics. While not wishing to be bound by theory, it is believed that the FBR to the electronics unit alone may be greater than the FBR to the sensing unit alone, due to the electronics unit's greater mass, for example. Accordingly, separation of the sensing and electronics units effectively reduces the FBR to the sensing unit and results in improved device function. As described elsewhere herein, the architecture and/or composition of the sensing unit (e.g., inclusion of a bioactive releasing membrane with certain bioactive agents) can be implemented to further reduce the foreign body response to the tethered sensing unit.

In another example, an analyte sensor is designed with separate electronics and sensing units, wherein the sensing unit is inductively coupled to the electronics unit. In this example, the electronics unit provides power to the sensing unit and/or enables communication of data therebetween. FIGS. 3F and 3G illustrate exemplary systems that employ inductive coupling between an electronics unit 52 and a sensing unit 58.

FIG. 3F is a side view of one example of an implanted sensor inductively coupled to an electronics unit within a functionally useful distance on the host's skin. FIG. 3F illustrates a sensing unit 58, including a sensing mechanism 36, biointerface membrane 68 and bioactive releasing membrane 70 at the distal end 37 of sensor 34, and small electronics chip 216 implanted below the host's skin 212, within the host's tissue 210. In this example, the majority of the electronics associated with the sensor are housed in an electronics unit 52 (also referred to as a mounting unit) located within suitably close proximity on the host's skin. The electronics unit 52 is inductively coupled to the small electronics chip 216 on the sensing unit 58 and thereby transmits power to the sensor and/or collects data, for example. The small electronics chip 216 coupled to the sensing unit 58 provides the necessary electronics to provide a bias potential to the sensor, measure the signal output, and/or other necessary requirements to allow the mechanism of the sensing unit 58 to function (e.g., chip 216 can include an ASIC (application specific integrated circuit), antenna, and other necessary components appreciated by one skilled in the art).

In yet another example, the implanted sensor additionally includes a capacitor to provide necessary power for device function. A portable scanner (e.g., wand-like device) is used to collect data stored on the circuit and/or to recharge the device.

In general, inductive coupling, as described herein, enables power to be transmitted to the sensor for continuous power, recharging, and the like. Additionally, inductive coupling utilizes appropriately spaced and oriented antennas (e.g., coils) on the sensing unit and the electronics unit so as to efficiently transmit/receive power (e.g., current) and/or data communication therebetween. One or more coils in each of the sensing and electronics unit can provide the necessary power induction and/or data transmission.

In this example, the sensing mechanism can be, for example, a wire-based sensor as described in more detail with reference to FIGS. 2A and 2B and as described in U.S. Pat. Pub. No. 2006/0020187, or a planar substrate-based sensor such as described in U.S. Pat. No. 6,175,752, to Say et al. and U.S. Pat. No. 5,779,665, to Mastrototaro et al., all of which are incorporated herein by reference in their entirety. The biointerface membrane 68 can be any suitable biointerface as described in more detail elsewhere herein, for example, a layer of porous biointerface membrane material, a mesh cage, and the like. In one exemplary example, the biointerface membrane 68 is a single- or multi-layer sheet (e.g., pocket) of porous membrane material, such as ePTFE, in which the sensing mechanism 36 is incorporated.

FIG. 3G is a side view of on example of an implanted sensor inductively coupled to an electronics unit implanted in the host's tissue at a functionally useful distance. FIG. 3G illustrates a sensing unit 58 and an electronics unit 52 similar to that described with reference to FIG. 3F, above, however both are implanted beneath the host's skin in a suitably close proximity.

In general, it is believed that when the electronics unit 52, which carries the majority of the mass of the implantable device, is separate from the sensing unit 58, a lesser foreign body response will occur surrounding the sensing unit (e.g., as compared to a device of greater mass, for example, a device including certain electronics and/or power supply). Thus, the configuration of the sensing unit, including a biointerface membrane and/or a bioactive releasing membrane, can be optimized to minimize and/or modify the host's tissue response, for example with minimal mass as described in more detail elsewhere.

### Biointerface Membrane/Layer

In one example, the sensor includes a porous material disposed over some portion thereof, which modifies the host's tissue response to the sensor. In some examples, the porous material surrounding the sensor advantageously enhances and extends sensor performance and lifetime by slowing or reducing cellular migration to the sensor and associated degradation that would otherwise be caused by cellular invasion if the sensor were directly exposed to the in vivo environment. Alternatively, the porous material can provide stabilization of the sensor via tissue ingrowth into the porous material in the long term. Suitable porous materials include silicone, polytetrafluoroethylene, expanded polytetrafluoroethylene, polyethylene-co-tetrafluoroethylene, polyolefin, polyester, polyalkylcarbonate, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), polybutylene terephthalate (PBT), polymethylmethacrylate (PMMA), polyether ether ketone (PEEK), polyamides, polyurethanes, polyurethane urea copolymer, cellulosic polymers, poly(ethylene oxide), poly(propylene oxide) and copolymers and blends thereof, polysulfones and block copolymers thereof including, for example, di-block, tri-block, alternating, random and graft copolymers, as well as metals, ceramics, cellulose, hydrogel polymers, polyethylene vinyl acetate (EVA), poly(2-hydroxyethyl methacrylate, pHEMA), hydroxyethyl methacrylate, (HEMA), polyacrylonitrile-polyvinyl chloride (PAN-PVC), high density polyethylene, acrylic copolymers, nylon, polyvinyl difluoride, polyanhydrides, poly(l-lysine), poly(L-lactic acid), hydroxyethylmetharcrylate, hydroxyapeptite, alumina, zirconia, carbon fiber, aluminum, calcium phosphate, titanium, titanium alloy, nintinol, stainless steel, and CoCr alloy, or the like, such as are described in U.S. Pat. No. 7,875,293, filed May 10, 2004 and entitled, “BIOINTERFACE MEMBRANES INCORPORATING BIOACTIVE AGENTS” and U.S. Pat. No. 7,192,450, filed Aug. 22, 2003 and entitled “POROUS MEMBRANES FOR USE WITH IMPLANTABLE DEVICES.”

In some examples, the porous material surrounding the sensor provides unique advantages in vivo (e.g., one to 14 days) that can be used to enhance and extend sensor performance and lifetime. However, such materials can also provide advantages in the long term too (e.g., greater than 14 days). Particularly, the in vivo portion of the sensor (the portion of the sensor that is implanted into the host's tissue) is encased (partially or fully) in a porous material. The porous material can be wrapped around the sensor (for example, by wrapping the porous material around the sensor or by inserting the sensor into a section of porous material sized to receive the sensor). Alternately, the porous material can be deposited on the sensor (for example, by electrospinning of a polymer directly thereon). In yet other alternative examples, the sensor is inserted into a selected section of porous biomaterial. Other methods for surrounding the in vivo portion of the sensor with a porous material can also be used as is appreciated by one skilled in the art.

The porous material surrounding the sensor advantageously slows or reduces cellular migration to the sensor and associated degradation that would otherwise be caused by cellular invasion if the sensor were directly exposed to the in vivo environment. Namely, the porous material provides a barrier that makes the migration of cells towards the sensor more tortuous and therefore slower. It is believed that this reduces or slows the sensitivity loss normally observed overtime.

In an example wherein the porous material is a high oxygen solubility material, such as porous silicone, the high oxygen solubility porous material surrounds some of or the entire in vivo portion of the sensor. In some examples, a lower ratio of oxygen-to-glucose can be sufficient to provide excess oxygen by using a high oxygen soluble domain (for example, a silicone- or fluorocarbon-based material) to enhance the supply/transport of oxygen to the enzyme membrane and/or electroactive surfaces. It is believed that some signal noise normally seen by a conventional sensor can be attributed to an oxygen deficit. Silicone has high oxygen permeability, thus promoting oxygen transport to the enzyme layer. By enhancing the oxygen supply through the use of a silicone composition, for example, glucose concentration can be less of a limiting factor. In other words, if more oxygen is supplied to the enzyme and/or electrochemically active surfaces, then more glucose can also be supplied to the enzyme without creating an oxygen rate-limiting excess. While not being bound by any particular theory, it is believed that silicone materials provide enhanced bio-stability when compared to other polymeric materials such as polyurethane.

In another example, the porous material further comprises a bioactive agent that releases upon insertion. In examples, the porous structure provides access for glucose permeation while allowing bioactive agent release/elute. In examples, as the bioactive agent releases/elutes from the porous structure, glucose transport may increase, for example, so as to offset any attenuation of glucose transport from the aforementioned immune response factors.

When used herein, the terms “membrane” and “matrix” are meant to be interchangeable. In these examples, the aforementioned porous material is a biointerface membrane comprising a first domain that includes an architecture, including cavity size, configuration, and/or overall thickness, that modifies the host's tissue response, for example, by creating a fluid pocket, encouraging vascularized tissue ingrowth, disrupting downward tissue contracture, resisting fibrous tissue growth adjacent to the device, and/or discouraging barrier cell formation. The biointerface membrane in examples covers at least the sensing mechanism of the sensor and can be of any shape or size, including uniform, asymmetrically, or axi-symmetrically covering or surrounding a sensing mechanism or sensor.

A second domain of the biointerface membrane is optionally provided that is impermeable to cells and/or cell processes. A bioactive agent is optionally provided that is incorporated into the at least one of the first domain, the second domain, the sensing membrane, or other part of the implantable device, wherein the bioactive agent is configured to modify a host tissue response. In examples, the biointerface includes a bioactive agent, the bioactive agent being incorporated into at least one of the first and second domains of the biointerface membrane, or into the device and adapted to diffuse through the first and/or second domains, in order to modify the tissue response of the host to the membrane.

Due to the small dimension(s) of the sensor (sensing mechanism) of the present disclosure, some conventional methods of porous membrane formation and/or porous membrane adhesion are inappropriate for the formation of the biointerface membrane onto the sensor as described herein. Accordingly, the following examples exemplify systems and methods for forming and/or adhering a biointerface membrane onto a small structured sensor as defined herein. For example, the biointerface membrane or release membrane of the present disclosure can be formed onto the sensor using techniques such as electrospinning, molding, weaving, direct-writing, lyophilizing, wrapping, and the like.

In examples wherein the biointerface is directly-written onto the sensor, a dispenser dispenses a polymer solution using a nozzle with a valve, or the like, for example as described in U.S. Pat. Pub. No. 2004/0253365. In general, a variety of nozzles and/or dispensers can be used to dispense a polymeric material to form the woven or non-woven fibers of the biointerface membrane.

### Bioactive Releasing Membrane/Layer—Inflammatory Response Control

In general, the inflammatory response to biomaterial implants can be divided into two phases. The first phase consists of mobilization of mast cells and then infiltration of predominantly polymorphonuclear (PMN) cells. This phase is termed the acute inflammatory phase. Over the course of days to weeks, chronic cell types that comprise the second phase of inflammation replace the PMNs. Macrophage and lymphocyte cells predominate during this phase. While not wishing to be bound by any particular theory, it is believed that restricting vasodilation and/or blocking pro-inflammatory signaling, short-term stimulation of vascularization, or short-term inhibition of scar formation or barrier cell layer formation, provides protection from scar tissue formation and/or reduces acute inflammation, thereby providing a stable platform for sustained maintenance of the altered foreign body response, for example.

Accordingly, bioactive intervention can modify the foreign body response in the early weeks of foreign body capsule formation and alter the extended behavior of the foreign body capsule. Additionally, it is believed that in some circumstances the biointerface membranes of the present disclosure can benefit from bioactive intervention to overcome sensitivity of the membrane to implant procedure, motion of the implant, or other factors, which are known to otherwise cause inflammation, scar formation, and hinder device function in vivo.

In general, bioactive agents that are believed to modify tissue response include anti-inflammatory agents, anti-infective agents, anti-proliferative agents, anti-histamine agents, anesthetics, inflammatory agents, growth factors, angiogenic (growth) factors, adjuvants, immunosuppressive agents, antiplatelet agents, anticoagulants, ACE inhibitors, cytotoxic agents, anti-barrier cell compounds, vascularization compounds, anti-sense molecules, and the like. In some examples, preferred bioactive agents include S1P (Sphingosine-1-phosphate), Monobutyrin, Cyclosporin A, Anti-thrombospondin-2, Rapamycin (and its derivatives), NLRP3 inflammasome inhibitors such as MCC950, and Dexamethasone. However, other bioactive agents, biological materials (for example, proteins), or even non-bioactive substances can incorporated into the membranes of the present disclosure.

Bioactive agents suitable for use in the present disclosure are loosely organized into two groups: anti-barrier cell agents and vascularization agents. These designations reflect functions that are believed to provide short-term solute transport through the one or more membranes of the presently disclosed sensor, and additionally extend the life of a healthy vascular bed and hence solute transport through the one or more membranes long term in vivo. However, not all bioactive agents can be clearly categorized into one or other of the above groups; rather, bioactive agents generally comprise one or more varying mechanisms for modifying tissue response and can be generally categorized into one or both of the above-cited categories.

### Anti-Barrier Cell Agents

Generally, anti-barrier cell agents include compounds exhibiting effects on macrophages and foreign body giant cells (FBGCs). It is believed that anti-barrier cell agents prevent closure of the barrier to solute transport presented by macrophages and FBGCs at the device-tissue interface during FBC maturation.

Anti-barrier cell agents generally include mechanisms that inhibit foreign body giant cells and/or occlusive cell layers. For example, Super Oxide Dismutase (SOD) Mimetic, which utilizes a manganese catalytic center within a porphyrin like molecule to mimic native SOD and effectively remove superoxide for long periods, thereby inhibiting FBGC formation at the surfaces of biomaterials in vivo, is incorporated into a biointerface membrane or release membrane of a preferred example.

Anti-barrier cell agents can include anti-inflammatory and/or immunosuppressive mechanisms that affect early FBC formation. Cyclosporine, which stimulates very high levels of neovascularization around biomaterials, can be incorporated into a biointerface membrane (see U.S. Pat. No. 5,569,462, to Martinson et al.), or release membrane of a preferred example.

In examples, dexamethasone, dexamethasone salts, or dexamethasone derivatives in particular, dexamethasone acetate, which, for example, abates the intensity of the FBC response at the device-tissue interface, is incorporated into the bioactive releasing membrane 70. In another example, a combination of dexamethasone and dexamethasone acetate is incorporated into the bioactive releasing membrane 70. In another example, dexamethasone and/or dexamethasone acetate combined with one or more other anti-inflammatory and/or immunosuppressive agents is incorporated into the bioactive releasing membrane 70. Alternatively, Rapamycin, which is a potent specific inhibitor of some macrophage inflammatory functions, can be incorporated into the release membrane alone or in combination with dexamethasone, dexamethasone salts, dexamethasone derivatives in particular, dexamethasone acetate.

Other suitable medicaments, pharmaceutical compositions, therapeutic agents, or other desirable substances can be incorporated into the bioactive releasing membrane 70 of the present disclosure, including, but not limited to, anti-inflammatory agents, anti-infective agents, necrosing agents, and anesthetics.

Generally, anti-inflammatory agents reduce acute and/or chronic inflammation adjacent to the implant, in order to decrease the formation of a FBC capsule to reduce or prevent barrier cell layer formation. Suitable anti-inflammatory agents include but are not limited to, for example, nonsteroidal anti-inflammatory drugs (NSAIDs) such as acetometaphen, aminosalicylic acid, aspirin, celecoxib, choline magnesium trisalicylate, diclofenac potassium, diclofenac sodium, diflunisal, etodolac, fenoprofen, flurbiprofen, ibuprofen, indomethacin, interleukin (IL)-10, IL-6 mutein, anti-IL-6 iNOS inhibitors (for example, L-NAME or L-NMDA), Interferon, ketoprofen, ketorolac, leflunomide, melenamic acid, mycophenolic acid, mizoribine, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, paclitaxel, tacrolimus, tranilast, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, NLRP3 inflammasome inhibitors such as MCC950, dexamethasone, and dexamethasone acetate.

Generally, immunosuppressive and/or immunomodulatory agents interfere directly with several key mechanisms necessary for involvement of different cellular elements in the inflammatory response. Suitable immunosuppressive and/or immunomodulatory agents include anti-proliferative, cell-cycle inhibitors, (for example, paclitaxol (e.g., Sirolimus), cytochalasin D, infiximab), taxol, actinomycin, mitomycin, thospromote VEGF, estradiols, NO donors, QP-2, tacrolimus, tranilast, actinomycin, everolimus, methothrexate, mycophenolic acid, angiopeptin, vincristing, mitomycine, statins, C MYC antisense, sirolimus (and analogs), RestenASE, 2-chloro-deoxyadenosine, PCNA Ribozyme, batimstat, prolyl hydroxylase inhibitors, PPARy ligands (for example troglitazone, rosiglitazone, pioglitazone), halofuginone, C-proteinase inhibitors, probucol, BCP671, EPC antibodies, catchins, glycating agents, endothelin inhibitors (for example, Ambrisentan, Tesosentan, Bosentan), Statins (for example, Cerivasttin), E. coli heat-labile enterotoxin, and advanced coatings.

Generally, anti-infective agents are substances capable of acting against infection by inhibiting the spread of an infectious agent or by killing the infectious agent outright, which can serve to reduce immuno-response without inflammatory response at the implant site. Anti-infective agents include, but are not limited to, anthelmintics (mebendazole), antibiotics including aminoclycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin; clindamycin; colistimethate sodium; polymyxin b sulfate; vancomycin; antivirals including acyclovir, amantadine, didanosine, efavirenz, foscarnet, ganciclovir, indinavir, lamivudine, nelfinavir, ritonavir, saquinavir, silver, stavudine, valacyclovir, valganciclovir, zidovudine; quinolones (ciprofloxacin, levofloxacin); sulfonamides (sulfadiazine, sulfisoxazole); sulfones (dapsone); furazolidone; metronidazole; pentamidine; sulfanilamidum crystallinum; gatifloxacin; and sulfamethoxazole/trimethoprim.

Generally, necrosing agents are any drug that causes tissue necrosis or cell death. Necrosing agents include cisplatin, BCNU, taxol or taxol derivatives, and the like.

### Vascularization Agents

Generally, vascularization agents include substances with direct or indirect angiogenic properties. In some cases, vascularization agents may additionally affect formation of barrier cells in vivo. By indirect angiogenesis, it is meant that the angiogenesis can be mediated through inflammatory or immune stimulatory pathways. It is not fully known how agents that induce local vascularization indirectly inhibit barrier-cell formation; however it is believed that some barrier-cell effects can result indirectly from the effects of vascularization agents.

Vascularization agents include mechanisms that promote neovascularization around the membrane and/or minimize periods of ischemia by increasing vascularization close to the device-tissue interface. Sphingosine-1-Phosphate (S1P), which is a phospholipid possessing potent angiogenic activity, is incorporated into a biointerface membrane or release membrane of a preferred example. Monobutyrin, which is a potent vasodilator and angiogenic lipid product of adipocytes, is incorporated into a biointerface membrane or release membrane of a preferred example. In another example, an anti-sense molecule (for example, thrombospondin-2 anti-sense), which increases vascularization, is incorporated into a biointerface membrane or release membrane.

Vascularization agents can include mechanisms that promote inflammation, which is believed to cause accelerated neovascularization in vivo. In one example, a xenogenic carrier, for example, bovine collagen, which by its foreign nature invokes an immune response, stimulates neovascularization, and is incorporated into a biointerface membrane or release membrane of the present disclosure. In another example, Lipopolysaccharide, which is a potent immunostimulant, is incorporated into a biointerface membrane or release membrane. In another example, a protein, for example, a bone morphogenetic protein (BMP), which is known to modulate bone healing in tissue, is incorporated into a biointerface membrane or release membrane of a preferred example.

Generally, angiogenic agents are substances capable of stimulating neovascularization, which can accelerate and sustain the development of a vascularized tissue bed at the device-tissue interface. Angiogenic agents include, but are not limited to, copper ions, iron ions, tridodecylmethylammonium chloride, Basic Fibroblast Growth Factor (bFGF), (also known as Heparin Binding Growth Factor-II and Fibroblast Growth Factor II), Acidic Fibroblast Growth Factor (aFGF), (also known as Heparin Binding Growth Factor-I and Fibroblast Growth Factor-1), Vascular Endothelial Growth Factor (VEGF), Platelet Derived Endothelial Cell Growth Factor BB (PDEGF-BB), Angiopoietin-1, Transforming Growth Factor Beta (TGF-Beta), Transforming Growth Factor Alpha (TGF-Alpha), Hepatocyte Growth Factor, Tumor Necrosis Factor-Alpha (TNF-Alpha), Placental Growth Factor (PLGF), Angiogenin, Interleukin-8 (IL-8), Hypoxia Inducible Factor-I (HIF-1), Angiotensin-Converting Enzyme (ACE) Inhibitor Quinaprilat, Angiotropin, Thrombospondin, Peptide KGHK, Low Oxygen Tension, Lactic Acid, Insulin, Copper Sulphate, Estradiol, prostaglandins, cox inhibitors, endothelial cell binding agents (for example, decorin or vimentin), glenipin, hydrogen peroxide, nicotine, and Growth Hormone.

Generally, pro-inflammatory agents are substances capable of stimulating an immune response in host tissue, which can accelerate or sustain formation of a mature vascularized tissue bed. For example, pro-inflammatory agents are generally irritants or other substances that induce chronic inflammation and chronic granular response at the implantation-site. While not wishing to be bound by theory, it is believed that formation of high tissue granulation induces blood vessels, which supply an adequate or rich supply of analytes to the device-tissue interface. Pro-inflammatory agents include, but are not limited to, xenogenic carriers, Lipopolysaccharides, S. aureus peptidoglycan, and proteins.

Other substances that can be incorporated into membranes of the present disclosure include various pharmacological agents, excipients, and other substances well known in the art of pharmaceutical formulations.

Although the bioactive agent in some examples is incorporated into the biointerface membrane or release membrane and/or implantable device, in some examples the bioactive agent can be administered concurrently with, prior to, or after implantation of the device systemically, for example, by oral administration, or locally, for example, by subcutaneous injection near the implantation site. A combination of bioactive agent incorporated in the biointerface membrane and bioactive agent administration locally and/or systemically can be preferred in certain examples.

In one example, the bioactive releasing membrane 70 functions as the biointerface membrane. In another example, the bioactive releasing membrane 70 is chemically distinct from the biointerface membrane 68, or no biointerface membrane 68 is used. In such examples, one or more bioactive agents are incorporated into the bioactive releasing membrane 70 or both the biointerface membrane 68 and the bioactive releasing membrane 70.

Generally, numerous variables can affect the pharmacokinetics of bioactive agent release. The bioactive agents of the present disclosure can be optimized for short- and/or extended release. In some examples, the bioactive agents of the present disclosure are designed to aid or overcome factors associated with short-term effects (for example, acute inflammation) of the foreign body response, which can begin as early as the time of implantation and extend up to about one month after implantation. In some examples, the bioactive agents of the present disclosure are designed to aid or overcome factors associated with extended effects, for example, chronic inflammation, barrier cell layer formation, or build-up of fibrotic tissue of the foreign body response, which can begin as early as about one week after implantation and extend for the life of the implant, for example, months to years. In some examples, the bioactive agents of the present disclosure combine short- and extended release to exploit the benefits of both. U.S. Pat. Pub. No. 2005/0031689, to Shults et al. discloses a variety of systems and methods for release of the bioactive agents.

The amount of loading of the bioactive agent into the release membrane can depend upon several factors. For example, the bioactive agent dosage and duration can vary with the intended use of the release membrane, for example, cell transplantation, analyte measuring-device, and the like; differences among hosts in the effective dose of bioactive agent; location and methods of loading the bioactive agent; and release rates associated with bioactive agents and optionally their chemical composition and/or bioactive agent loading. Therefore, one skilled in the art will appreciate the variability achieving a reproducible and controlled release of the one or more bioactive agents, at least for the reasons described above. U.S. Pat. Pub. No. 2005/0031689, to Shults et al. that discloses a variety of systems and methods for loading of the bioactive agents.

In examples, multiple layers or discrete or semi-discrete rings or bands of the bioactive releasing membrane are employed to specifically tailor the release of the bioactive agent for the intended sense of life. Thus, in examples, two or more layers of the multilayer bioactive releasing membrane differs in one or more aspects, for example: of hydrophobicity/hydrophilicity content or ratio of the segments of a soft-hard segmented polymer or copolymer; compositional makeup or weight percent of two or more different polymers or copolymers or blends of different polymers and/or copolymers in each layer or their vertical or horizontal distribution in one or more layers; bioactive loading and/or distribution (vertically or longitudinally within the coated membrane) in each layer; membrane thickness of each layer; composition and loading amount of two or more distinct bioactive agents (e.g., a neutral, derivative and/or salt form or a primary form and derivative form of the bioactive agent); the solvent system used to cast or deposit or dip coat the individual bioactive releasing membrane layers; and the relative position(s) (continuous, semicontinuous, or noncontinuous positioning) of the bioactive releasing membrane layers along the length of the sensor.

Bioactive Releasing Membrane/Layer—Formation onto the Sensor

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, polarimetric, potentiometric, calorimetric, radiometric, immunochemical, or like elements.

Although some of the description that follows is directed at glucose-measuring devices, including the described membrane systems and methods for their use, these membrane systems are not limited to use in devices that measure or monitor glucose. These membrane systems are suitable for use in any of a variety of devices, including, for example, devices that detect and quantify other analytes present in biological fluids (e.g. cholesterol, amino acids, alcohol, galactose, and lactate), cell transplantation devices (see, for example, U.S. Pat. Nos. 6,015,572, 5,964,745, and 6,083,523), drug delivery devices (see, for example, U.S. Pat. Nos. 5,458,631, 5,820,589, and 5,972,369), and the like, which are incorporated herein by reference in their entireties for their teachings of membrane systems.

Suitable bioactive releasing membranes are those membranes which provide a therapeutically effective amount and release rate of bioactive agent beginning with the insertion of the sensor and throughout the life of the sensor. In one example, the bioactive releasing membrane in combination with an amount of bioactive agent provides for extending the useful life of the sensor when compared to an equivalent sensor the bioactive releasing membrane without the bioactive agent (or compared to the absence of the bioactive releasing membrane and bioactive agent). As used herein a therapeutically effective amount of the bioactive agent is an amount capable of inducing an intended therapeutic effect. An intended therapeutic effect is one that can be readily determined using conventional diagnostic methods. For example, an intended therapeutic effect encompasses suppressing unwanted foreign body response to an implant (foreign body) including, but not limited to inflammation and/or fibrous capsule formation.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membrane.

Polymers with domains or segments that are functionalized to permit cross-linking can be made by methods known in the art. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thio groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In examples, from about 0.1% to about 15% w/w of cross-linking agent is added relative to the total dry weights of cross-linking agent and polymers added when blending the ingredients (in examples, about 1% to about 10%), including all ranges and subranges therebetween. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using any method known in the art (e.g., spraying, painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., 50-150° C.). Other suitable curing methods can include ultraviolet or gamma radiation, for example.

In one example, the weight of bioactive agent associated with the sensor is 1-120 μL, 2-110 μL, 3-100 μL, 4-90 μL, 5-80 μL, 6-70 μL, 7-60 μL, 8-50 μL, 9-40 μL, or 10-30 μL. In another example, the weight of two or more bioactive agents associated with the sensor, independently or collectively is 1-120 μL, 2-110 μL, 3-100 μL, 4-90 μL, 5-80 μL, 6-70 μL, 7-60 μL, 8-50 μL, 9-40 μL, or 10-30 μL.

In one example, the weight percent loading of bioactive agent in the bioactive releasing membrane 70 is about 10 weight percent to about 90 weight percent. In examples, the weight percent loading of bioactive agent in the bioactive releasing membrane 70 is 10%, 20%, 30%, 40%, 50%, 60%, 70%, 80%, or 90%, of the total weight of the bioactive releasing membrane plus bioactive agent (as a deposited membrane on a sensor). In examples, the weight percent loading of bioactive agent in the bioactive releasing membrane 70 is 30%, 40%, 50%, or 60%, of the total weight of the bioactive releasing membrane plus bioactive agent (as a deposited membrane on a sensor). Depending on the nature of the bioactive releasing membrane, for example, the ratio of hydrophobic/hydrophilic soft segments, the weight percent of the bioactive agent is chosen based on solubility/miscibility/dispersion of the bioactive agent with the bioactive releasing membrane and any solvent or solvent system used to dispense the bioactive releasing membrane and bioactive agent onto the sensor. Too high a loading of bioactive agent in a particular bioactive releasing membrane can result in precipitation of the bioactive agent, and/or poor coating quality. Too low a loading of bioactive agent in the bioactive releasing membrane can result in inefficient therapeutic effect over the intended lifetime of the sensor, which can manifest itself as poor signal-to-noise initially and/or prior to the designed end-of-life of the sensor, reduction or fluctuation of sensitivity of the sensor to the target analyte(s) shortly after insertion and/or prior to the designed end-of-life of the sensor, among other things.

In examples, the bioactive releasing membrane is configured to release, in weight percent, after insertion and up to the end of life of the sensor, at least 10%, 20%, 30%, 40%, 50%, 60%, 70%, 80%, 90%, up to and including 100% of the initial loading of the bioactive agent. In examples, the bioactive releasing membrane is configured to release, after insertion and up to the end of life of the sensor, between 60-90 weight percent of the bioactive agent. In another example, the bioactive releasing membrane is configured to release, after insertion and up to the end of life of the sensor, between 75-85 weight percent of the bioactive agent.

In one example, the bioactive releasing membrane of the present disclosure provides for release of the bioactive agent from the bioactive releasing membrane commensurate with a bolus amount of the bioactive agent. In another example, the bioactive releasing membrane of the present disclosure provides for release of the bioactive agent from the bioactive releasing membrane commensurate with a therapeutically effective amount of the bioactive agent. In examples, the bioactive releasing membrane of the present disclosure provides for release of the bioactive agent from the bioactive releasing membrane commensurate with a non-therapeutically effective amount where the non-therapeutically effective amount follows one or more of a release of a bolus amount or therapeutic amount of the bioactive agent.

In examples, the bioactive releasing membrane of the present disclosure provides for a bolus release of the bioactive agent essentially immediately upon insertion of the sensor for a first time period or range (for example, minutes, hours, days, weeks, etc.), the first time period or range initiated at a first time point (for example, a second or less) into the subject's soft tissue. In examples, the bioactive releasing membrane of the present disclosure provides for release of a bolus amount of the bioactive agent essentially immediately upon insertion of the sensor, for the first time period initiated at the first time point, into the subject's soft tissue followed by release of a therapeutically effective amount of the bioactive agent beginning at a second time point for a second time period, the second time period overlapping with or subsequent to the first time period. In examples, the second time point is subsequent to the first time point by at least 10 seconds, 30 seconds, 1 minute, 5 minutes, 10 minutes or more. In one example, the bioactive releasing membrane of the present disclosure provides for release of a bolus amount of the bioactive agent essentially immediately upon insertion of the sensor, for the first time period initiated at the first time period, into the subject's soft tissue followed by release of a therapeutically effective amount of the bioactive agent beginning at a second time point for a second time period, the second time period overlapping with or subsequent to the first time period, followed by a release of a non-therapeutically effective amount of the bioactive agent beginning at a third time point for a third time period, the third time period overlapping with or subsequent to the second time period. In examples, the third time point is subsequent to the second time point by at least 10 seconds, 30 seconds, 1 minute, 5 minutes, 10 minutes or more.

In examples, the bolus release of bioactive is combined with the release of a non-active pharmaceutical ingredient (non-API) such as a hydrophilic substance (zwitterion species, hydrogel particles or spheres) so as to modify the environment created in the tissue by the presence of the sensor's volume. While not wishing to be bound by theory, it is believed that hydrophilic substance attracts fluids to the environment, which can mitigate biofouling, slow down metabolic breakdown of the bioactive, and/or increase bioactive uptake into cells. Such release of non-API may facilitate the delay of the foreign body response and/or facilitate the release of the bioactive from the bioactive releasing membrane, among other benefits.

Release rates of the bioactive agent in any of the aforementioned first, second or third time periods can be the same or different. Release rates of the bioactive agent in any of the aforementioned first, second or third time periods can be configured to occur at a substantially constant rate or a variable rate (intermittent, periodic, and/or random) by modifying one or more of membrane chemistry, structure, and/or morphology, bioactive agent loading, bioactive chemistry, for example. Release rates (the concentration or amount of bioactive released over time) of the bioactive agent in any of the aforementioned time periods can be configured to change after implantation over time by modifying one or more of membrane chemistry, structure, and/or morphology, bioactive agent loading, bioactive chemistry, for example.

In one example, the release rate of the bioactive agent from the bioactive releasing membrane initially or during the first time period is greater than the release rate of the bioactive agent from the bioactive releasing membrane initially or during the second time period. In one example, the release rate of the bioactive agent from the bioactive releasing membrane initially or during the second time period is greater than the release rate of the bioactive agent from the bioactive releasing membrane initially or during the third time period. In one example, the release rate of the bioactive agent from the bioactive releasing membrane initially or during the first time period is greater than the release rate of the bioactive agent from the bioactive releasing membrane initially or during the second time period and the and release rate of the bioactive agent from the bioactive releasing membrane initially or during the second time period is greater than the release rate of the bioactive agent from the bioactive releasing membrane initially the third time period.

Suitable bioactive releasing membranes of the present disclosure capable of the aforementioned release rates and released amounts of the bioactive agents can be selected from silicone polymers, polytetrafluoroethylene, expanded polytetrafluoroethylene, polyethylene-co-tetrafluoroethylene, polyolefin, polyester, polyalkylester, polyalkylcarbonate, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), polyethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

A suitable bioactive releasing membrane is a polyurethane, or polyetherurethaneurea. Polyurethane is a polymer produced by the condensation reaction of a diisocyanate and a difunctional hydroxyl-containing material. A polyurethaneurea is a polymer produced by the condensation reaction of a diisocyanate and a difunctional amine-containing material. Exemplary diisocyanates include aliphatic diisocyanates containing from about 4 to about 8 methylene units. Diisocyanates containing cycloaliphatic moieties can also be useful in the preparation of the polymer and copolymer components of the bioactive releasing membranes of the present disclosure. The material that forms the basis of the hydrophobic matrix of the bioactive releasing membrane or its domains can be any of those known in the art as appropriate for use as membranes in continuous analyte sensor devices. In one example, the bioactive releasing membrane is different from the other membranes of the sensor system in that the bioactive releasing membrane is less sufficient in its permeability to relevant compounds, for example, to allow an glucose molecule to pass through the membrane.

Examples of other materials which can be used to make non-polyurethane type bioactive releasing membranes include vinyl polymers, polyethylene vinyl acetate, polyethylene vinyl acetate copolymers, polyethers, polyesters, polyalkylesters, polyamides, polysilicones poly(dialkylsiloxanes), poly(alkylarylsiloxanes), poly(diarylsiloxanes), polycarbosiloxanes, polyalkylcarbonate, polycarbonate, natural polymers such as cellulosic and protein-based materials, and mixtures, copolymers, or combinations thereof with or without the aforementioned polyurethane, or polyetherurethaneurea polymers.

In examples, the bioactive releasing membrane comprises a soft segment and a hard segment comprising urethane groups, urea groups, or a combination of urethane groups and urea groups. The soft segment can be two or more different polymer segments. The soft segment can comprise a hydrophobic block and a hydrophilic block. The soft segment can comprise polysiloxane, polyalkylether, polyalkylester, polyalkylcarbonate, polycarbonate, or polysiloxane-polyalkylether segmented blocks.

In examples, the soft segment comprising, independently, combinations of hydrophobic/hydrophilic portions, such as polyols (polyethylene oxides “PEO”, polyethylenepropylene oxides, poly tetrahydrofuran or polytetramethylene oxide, polyethers, polysiloxanes, polyamines, polysiloxane amine, polyester, polyalkylester, polyalkylcarbonate, polycarbonate and one or more independent hard segments, e.g. an aliphatic or aromatic diisocyanate such as norbornane diisocyanate (NBDI), isophorone diisocynate (IPDI), tolylene diisocynate (TDI), 1,3-phenylene diisocyanate (MPDI), trans-1,3-bis(isocynatomethyl) cyclohexane (1,3-H6XDI), bicyclohexylmethane-4,4′-diisocynate(HMDI), 4,4′-Diphenylmethane diisocynate (MDI), trans-1,4-bis(isocynatomethyl) cyclohexane (1,4-H6XDI), 1,4-cyclohexyl diisocynate (CHDI), 1,4-phenylene diisocynate (PPDI), 3,3′-Dimethyl-4,4′-biphenyldiisocyanate (TODI), 1,6-hexamethylene diisocyanate (HDI), or combinations thereof.

In examples, the bioactive releasing membrane can further comprise a chain extender. The chain extender, for example, can be a diol, a diamine, a silicon-hydride, or a multifunctional epoxide. Exemplary diols include aliphatic or aromatic low molecular weight diols, e.g., glycols, propylene glycol, diethylene glycol, and 1,4-butanediol, and other exemplary chain extenders include dialkylamines, e.g., ethylene diamine, 1,6-hexamethylenediamine, 4,4′-diaminodiphenylmethane, triethylenediamine, putrescine, and diaminopropane, or hydroxylamines.

In other examples, the bioactive releasing membrane further comprises one or more zwitterionic repeating units selected from the group consisting of cocamidopropyl betaine, oleamidopropyl betaine, octyl sulfobetaine, caprylyl sulfobetaine, lauryl sulfobetaine, myristyl sulfobetaine, palmityl sulfobetaine, stearyl sulfobetaine, betaine (trimethylglycine), octyl betaine, phosphatidylcholine, glycine betaine, poly(carboxybetaine), poly(sulfobetaine), and derivatives thereof. In another aspect, alone or in combination with any one of the previous aspects, the bioactive releasing membrane does not comprise zwitterionic groups only at the end of the polymer chain.

In examples, the one or more zwitterionic repeating units are derived from a monomer selected from the group consisting of:

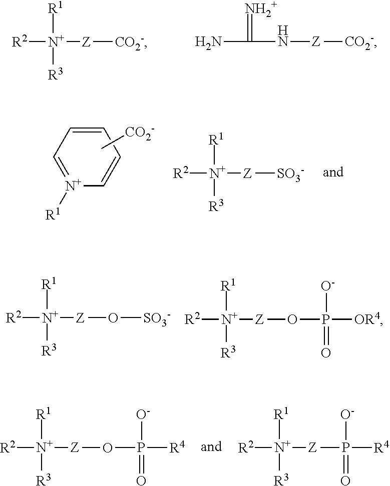

where Z is branched or straight chain alkyl, heteroalkyl, cycloalkyl, cycloheteroalkyl, aryl, or heteroaryl; R1 is H, alkyl, heteroalkyl, cycloalkyl, heterocycloalkyl, aryl, or heteroaryl; and R2, R3, and R4 are independently chosen from alkyl, heteroalkyl, cycloalkyl, heterocycloalkyl, aryl, or heteroaryl; and wherein one or more of R¹, R², R³, R⁴, and Z are substituted with a polymerization group are used as at least a portion of the bioactive releasing membrane.

In examples, the polymerization group is selected from alkene, alkyne, epoxide, lactone, amine, hydroxyl, isocyanate, carboxylic acid, anhydride, silane, halide, aldehyde, and carbodiimide. In another example, the one or more zwitterionic repeating units is at least about 1 wt. % based on the total weight of the polymer.

In one example, the least one bioactive agent is covalently associated with the bioactive releasing membrane. In another example, the at least one bioactive agent is ionically associated with the bioactive releasing membrane. In another example, the bioactive agent is a conjugate.

In another example, the at least one bioactive agent is a nitric oxide (NO) releasing molecule, polymer, or oligomer. In another aspect, alone or in combination with any one of the previous aspects, the nitric oxide releasing molecule is selected from N-diazeniumdiolates and S-nitrosothiols. In examples, the nitric oxide releasing molecule is covalently or noncovalently coupled to the polymer or oligomer. In examples, the N-diazeniumdiolate is of a structure: RR′N—N202, where R and R′ are independently alkyl, aryl, phenyl, alkylaryl, alkylphenyl, or functionalized N-alkylamino trialkoxy silane. In examples at least one of R and R′ groups of the N-diazeniumdiolate of a structure: RR′N—N202 are sufficiently lipophilic to remain in the hydrophobic region of the bioactive releasing membrane while providing a source of nitric oxide to the insertion site. In examples at least one of R and R′ are sufficiently functionalized to couple with the bioactive releasing membrane while providing a source of nitric oxide to the insertion site. In examples, the S-nitrosothiol is S-nitroso-glutathione (GSNO) or a S-nitrosothiol derivative of penicillamine.

In another example, the bioactive agent is a borate ester or boronate. In one example, the bioactive agent-borate ester or boranate is covalently coupled to the bioactive releasing membrane. In another example, the bioactive agent-borate ester or boranate is noncovalently coupled to the bioactive releasing membrane. In one example, the bioactive agent-borate ester or boranate is covalently coupled to the bioactive agent and covalently coupled to the bioactive releasing membrane. In another example, the bioactive agent-borate ester or boranate is covalently coupled to the bioactive agent and noncovalently coupled to the bioactive releasing membrane. In another example, the bioactive agent is a borate ester or boronate of dexamethasone, dexamethasone salts, or dexamethasone derivatives in particular, dexamethasone acetate, or dexamethasone acetate salt.

In another example, the bioactive agent is a conjugate comprising at least one cleavable linker by subcutaneous stimuli. In another example, the bioactive agent is a conjugate of dexamethasone, dexamethasone salts, or dexamethasone derivatives in particular, dexamethasone acetate, or dexamethasone acetate salt comprising at least one cleavable linker by subcutaneous stimuli. For example, the bioactive agent conjugate comprising at least one cleavable linker is cleaved by subcutaneous stimuli after insertion of the analyte sensor into the subcutaneous domain of the host. In one example, the subcutaneous stimuli is chemical attack by one or more members of the metzincin superfamily, matrix metalloproteinases (MMPs), or matrix metallopeptidases or matrixins, or any other protease. In examples, the MMP is a calcium-, or zinc-dependent endopeptidase, adamalysins, astacins, or serralysins.

In another example, the bioactive releasing membrane comprising the bioactive agent (alone or as a conjugate or associated with the bioactive releasing membrane) comprises a hydrophilic hydrogel, where the hydrophilic hydrogel is at least partly crosslinked and dissolvable in biological fluid. In another example, the bioactive releasing membrane comprising the bioactive agent (alone or as a conjugate) comprises a hydrophilic hydrogel associated with or coupled to dexamethasone, dexamethasone salts, or dexamethasone derivatives in particular, dexamethasone acetate, or dexamethasone acetate salt, where the hydrophilic hydrogel is at least partly crosslinked and dissolvable in biological fluid and provides for release of the dexamethasone, dexamethasone salts, or dexamethasone derivatives in particular, dexamethasone acetate, or dexamethasone acetate salt.

In examples, the hydrophilic hydrogel at least partially dissolves in biological fluid within 6 hours, 12 hours, 24 hours, 2 days, 3 days, 4 days, 5 days, 6 days, 7 days or more and provides for continuous, semicontinuous, or bolus release of the dexamethasone, dexamethasone salts, or dexamethasone derivatives in particular, dexamethasone acetate, or dexamethasone acetate salt. In examples, the hydrophilic hydrogel comprises hyaluronic acid (HA) crosslinked by divinyl sulfone or polyethylene glycol divinyl sulfone. In examples, the hydrophilic hydrogel comprises a hydrogel conjugate of the dexamethasone, dexamethasone salts, or dexamethasone derivatives in particular, dexamethasone acetate, or dexamethasone acetate salt.

In another aspect, the bioactive releasing membrane comprises silver nanoparticles or nanogels as the bioactive agent alone or in combination with dexamethasone, dexamethasone salts, or dexamethasone derivatives or mixtures thereof, in particular, dexamethasone acetate, or dexamethasone acetate salt. In one example, the nanoparticles are biodegradable. For example, the biodegradable polymeric nanoparticles comprises PLA, PLGA, PCL, PVL, PLLA, PDLA, PEO-b-PLA block copolymers, polyphosphoesters, or PEO-b-polypeptides comprising the at least one bioactive agent. In examples, the bioactive releasing membrane comprises copper and/or zinc nanoparticles or nanogels as the bioactive agent. The silver, copper or zinc nanoparticles/nanogels can be spatially distributed or dispersed throughout the bioactive releasing membrane where the spatial distribution or dispersion can be uniform or nonuniform, and/or vary vertically and/or horizontally in a gradient.

In examples a bacterial cellulose with self-assembled nanoparticles/nanogels of silver, zinc, or copper is used as the bioactive releasing membrane and provides for release of the dexamethasone, dexamethasone salts, or dexamethasone derivatives in particular, dexamethasone acetate, or dexamethasone acetate salt, alone or together with any one of the polyurethane/polyurethane urea membranes disclosed herein. In another example, chitosan oligosaccharide/poly(vinyl alcohol) nanoparticles/nanogels or nanofibers of silver, zinc, or copper is used as the bioactive releasing membrane and provides for release of the dexamethasone, dexamethasone salts, or dexamethasone derivatives in particular, dexamethasone acetate, or dexamethasone acetate salt.

In examples, the bioactive releasing membrane comprises biodegradable polymeric nanoparticles selected from PLA, PLGA, PCL, PVL, PLLA, PDLA, PEO-b-PLA block copolymers, polyphosphoesters, PEO-b-polypeptides, where the polymeric nanoparticles/nanogels comprise, covalently or noncovalently, associated dexamethasone, dexamethasone salts, or dexamethasone derivatives in particular, dexamethasone acetate, or dexamethasone acetate salt.

In another example, the bioactive releasing membrane comprises an organic and/or inorganic sol-gel, or organic-inorganic hybrid sol-gel, or poloxamer-based carrier providing for release of the dexamethasone, dexamethasone salts, ordexamethasone derivatives in particular, dexamethasone acetate, or dexamethasone acetate salt. In another example, the bioactive releasing membrane comprises a thermosensitive-controlled release hydrogel or poloxamer, for example, poly(ε-caprolactone)-poly(ethylene glycol)-poly(ε-caprolactone) hydrogel.

The aforementioned the bioactive releasing membrane in one example comprises a combination of at least one bioactive agent encapsulated in the bioactive releasing membrane and at least one bioactive agent covalently coupled to the bioactive releasing membrane. In another example, the bioactive releasing membrane comprises spatially distal drug depots of the at least one bioactive agent as a conjugate or as associated with the bioactive releasing membrane, as disclosed herein.

In another example, the bioactive releasing membrane comprises a hydrolytically degradable biopolymer comprising the at least one bioactive agent. In one example, the hydrolytically degradable biopolymer comprises a salicylic acid polyanhydride ester (Structure 1) capable of hydrolyzing to salicylic acid and adipic acid.

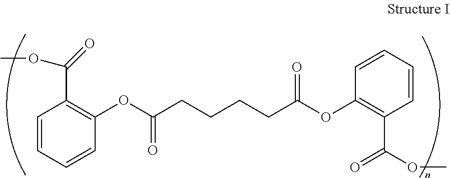

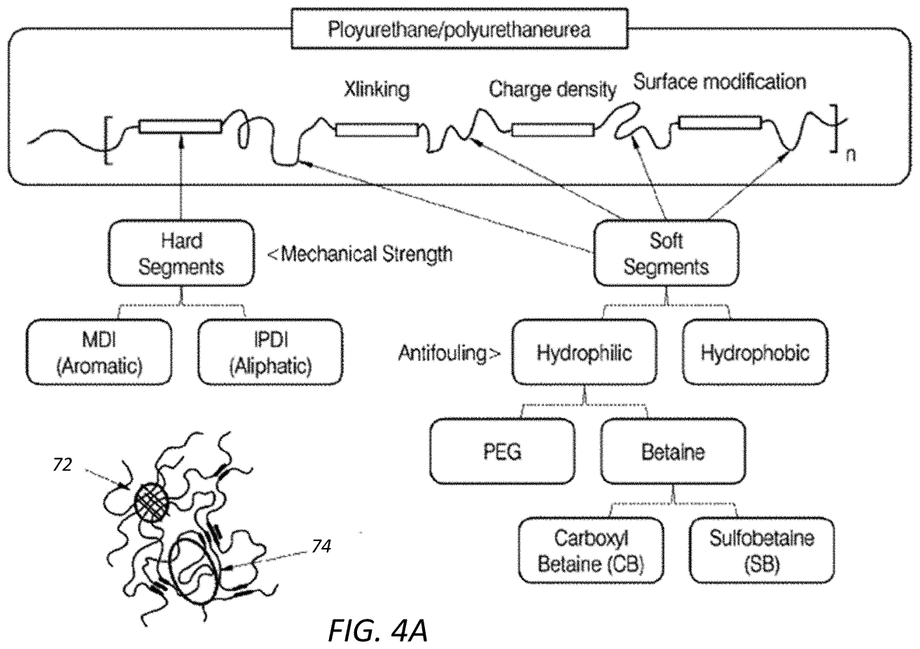

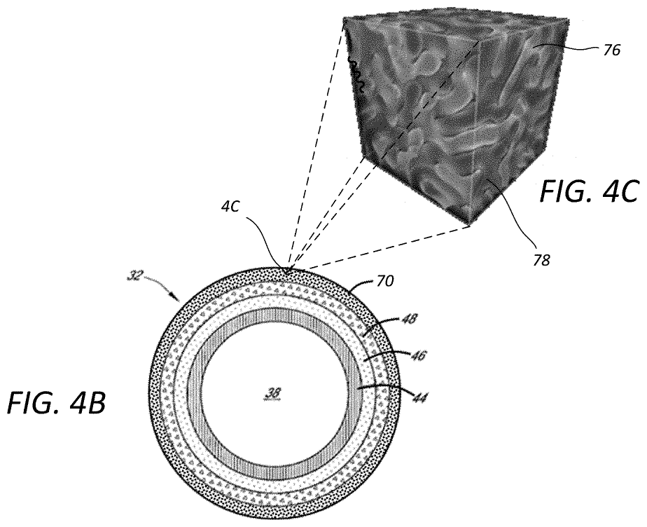

In one example, suitable bioactive releasing membranes 70 are hard-soft segmented polymers. With reference to FIG. 4A, an exemplary hard-soft segmented copolymer is depicted having a hard segment 72 where there is close association of polymer segments providing crystallinity or crystalline like structure and a soft segment 74 providing an amorphous or amorphous-like structure. In one example the bioactive releasing membrane 70 of the present disclosure is a hard-soft segmented copolymer 71 where the soft segment 74 comprises a hydrophilic polymer or hydrophilic polymer segment. In one example the bioactive releasing membrane 70 of the present disclosure is a hard-soft segmented copolymer 71 where the soft segment 74 comprises a hydrophilic polymer or hydrophilic polymer segment in combination with a hydrophobic polymer or hydrophobic polymer segment. With reference to FIG. 4B, 4C a hard-soft segmented copolymer where the soft segment 74 comprises a hydrophilic polymer or hydrophilic polymer segment in combination with a hydrophobic polymer or hydrophobic polymer segment is schematically shown as a three-dimensional volume 4C of bioactive releasing membrane 70 of sensing membrane 32, which depicts the arrangement of hydrophobic domains 76 and hydrophilic domains 78. Various confirmations and distributions of the hydrophobic domains and hydrophilic domains are envisioned depending on the relative concentrations of each domain and whether there is non-stoichiometric or stoichiometric amounts of each domain. In examples, the soft segment of the bioactive releasing membrane 70 comprises a hydrophilic segment, not including zero weight percent, and a hydrophobic segment, including zero weight percent.

In one example, the bioactive releasing membrane 70 comprises a hard-soft segmented polyurethane copolymer. In another example, the bioactive releasing membrane 70 comprises a hard-soft segmented polyurethane urea copolymer. In examples the bioactive releasing membrane 70 of the present disclosure is a hard-soft segmented polyurethane or polyurethane urea copolymer where the soft segment 74 comprises a hydrophilic polymer, or hydrophilic polymer segment in combination with a hydrophobic polymer or hydrophobic polymer segment. In examples the bioactive releasing membrane 70 of the present disclosure is a hard-soft segmented polyurethane or polyurethane urea copolymer blend where at least one of the individual polymers of the polymer blend comprises a soft segment 74 comprises a hydrophilic polymer or hydrophilic polymer segment in combination with a hydrophobic polymer or hydrophobic polymer segment. In examples the bioactive releasing membrane 70 of the present disclosure is a hard-soft segmented polyurethane or polyurethane urea copolymer blend, where at least one of the individual polymers of the polymer blend comprises a soft segment 74 comprises a hydrophilic polymer segment only and at least one polymer of the polymer blend comprises a soft segment comprising hydrophilic polymer segment in combination with a hydrophobic polymer or hydrophobic polymer segment.

In examples, the bioactive releasing membrane 70 comprises a hard-soft segmented polyurethane copolymer or polyurethane-urea copolymer comprising a pharmaceutical amount of the bioactive and providing for release of the bioactive having a release profile (bolus, bolus then controlled release, etc.). The bioactive can be dexamethasone ((11β,16α)-9-fluoro-11,17,21-trihydroxy-16-methylpregna-1,4-diene-3,20-dione), dexamethasone salts (e.g., sodium phosphate), or dexamethasone derivatives (or analogs) in particular, dexamethasone acetate; dexamethasone acetate salt; dexamethasone 17-propionate; dexamethasone enol-pyruvaldehyde; (Z)-2-((8S,9R,10S,11S,13S,14S,16R)-9-fluoro-11-hydroxy-10,13,16-trimethyl-3-oxo-3,6,7,8,9,10,11,12,13,14,15,16-dodecahydro-17H-cyclopenta[a]phenanthrene-17-ylidene)-2-hydroxyacetaldehyde; 2-((10R,13S,16S,17R)-11,17-dihydroxy-10,13,16-trimethyl-4,9,10,11,12,13,14,15,16,17-decahydrospiro[cyclopenta[a]phenanthren-3,2′-[1,3]dioxolan]-17-yl)-2-oxoethyl acetate; (8S,9R,10R,11S,13S,14S,16R,17R)-9-fluoro-1,11,17-trihydroxy-17-(2-hydroxyacetyl)-10,13,16-trimethyl-1,2,6,7,8,9,10,11,12,13,14,15,16,17-tetradecahydro-3H-cyclopenta[a]phenanthren-3-one; dexamethasone glyoxal; or 2-((8S,9R,10S,11S,13S,14S,16R,17R)-9-fluoro-11,17-dihydroxy-10,13,16-trimethyl-3-oxo-6,7,8,9,10,11,12,13,14,15,16,17-dodecahydro-3H-cyclopenta[a]henanthrene-17-yl)-2-oxoacetic acid.

In some examples, the hard segment of the copolymer may have an average or number average molecular weight of from about 160 daltons (DA) to about 10,000 DA, or from about 200 DA to about 2,000 DA, including all ranges and subranges therebetween. In some examples, the average molecular weight or number average molecular weight of the soft segment may be from about 200 DA to about 100,000 DA, or from about 500 DA to about 500,000 DA, or from about 5,000 DA to about 20,000 DA, including all ranges and subranges therebetween.

In some examples, a base polymer the bioactive releasing membrane has an average molecular weight or number average molecular weight from about 200 DA to about 10,000 DA, from about 10,000 DA to about 50,000 DA, from about 50,000 DA to about 100,000 DA, from about 100,000 DA to about 150,000 DA, from about 150,000 DA to about 250,000 DA, or from about 250,000 DA to about 500,000 DA, including all ranges and subranges therebetween.

In examples, aliphatic or aromatic diisocyanates are used to prepare the hard segment 72 of bioactive releasing membrane 70. In examples, the aliphatic or aromatic diisocyanate used to provide the hard segment 72 of bioactive releasing membrane 70 is norbornane diisocyanate (NBDI), isophorone diisocynate (IPDI), tolylene diisocynate (TDI), 1,3-phenylene diisocyanate (MPDI), trans-1,3-bis(isocynatomethyl) cyclohexane (1,3-H6XDI), bicyclohexylmethane-4,4′-diisocynate(HMDI), 4,4′-Diphenylmethane diisocynate (MDI), trans-1,4-bis(isocynatomethyl) cyclohexane (1,4-H6XDI), 1,4-cyclohexyl diisocynate (CHDI), 1,4-phenylene diisocynate (PPDI), 3,3′-Dimethyl-4,4′-biphenyldiisocyanate (TODI), 1,6-hexamethylene diisocyanate (HDI), or combinations thereof.

In examples, the soft segment 74 of the hard-soft segmented polyurethane or polyurethane urea copolymer comprises polysiloxane or copolymer thereof. In examples, the soft segment 74 of the hard-soft segmented polyurethane or polyurethane urea copolymer comprises poly(dialkyl)siloxane, poly(diphenyl)siloxane, poly(alkylphenyl)siloxane or copolymer thereof. In examples, the soft segment 74 of the hard-soft segmented polyurethane or polyurethane urea copolymer comprises poly(alkyl)oxy polymer, poly(alkylene)oxide, or copolymers thereof. In examples, the soft segment 74 of the hard-soft segmented polyurethane or polyurethane urea copolymer comprises poly(alkyl)oxide, poly(ethylene)oxide, poly(propylene)oxide, poly(ethylene-propylene) oxide, poly(tetraalkylene)oxide, poly(tetramethylene)oxide polymer or copolymers or blends thereof. The soft segments can be comprised of hydrophilic and/or hydrophobic oligomers of, for example, polyalkylene glycols, polyalkylcarbonate, polycarbonates, polyesters, polyethers, polyvinylalcohol, polyvinypyrrolidone, polyoxazoline, and the like.

In examples, the soft segment 74 of the hard-soft segmented polyurethane or polyurethane urea copolymer comprises polysiloxane or copolymer thereof and poly(alkylene)oxy polymer or copolymers thereof. In examples, the soft segment 74 of the hard-soft segmented polyurethane or polyurethane urea copolymer comprises poly(dialkyl)siloxane, poly(diphenyl)siloxane, poly(alkylphenyl)siloxane or copolymer and poly(alkyl)oxide, poly(ethylene) oxide, poly(propylene)oxide, poly(ethylene-propylene) oxide, poly(tetraalkylene)oxide, poly(tetramethylene)oxide polymer or copolymers or blends thereof.

In one example, the bioactive releasing membrane 70 has a hydrophilic segments having a static contact angle greater than 90 degrees. In one example the bioactive releasing membrane 70 has hydrophobic segments with a static contact angle of less than 90 degrees. Examples of hydrophilic polymers suitable for at least a portion of the soft segment of bioactive releasing membrane 70 so as to provide a static contact angle of 90 degrees or more include, but are not limited to, polyvinylpyrrolidone, polyvinylpyridine, proteins, cellulose, polyether, polyetherimine. Examples of hydrophobic polymers suitable for at least a portion of the soft segment of bioactive releasing membrane 70 so as to provide a static contact angle of less than 90 degrees include, but not limited to polyurethane, silicone, polyurethaneurea, polyester, polyamides, polyalkylcarbonate, polycarbonate, and copolymer thereof.

At least a portion of a surface of the biointerface/bioactive releasing membrane can be hydrophobic as measured by contact angle. For example, the biointerface/bioactive releasing membrane can have a contact angle of from about 90° to about 160°, from about 95 to about 155°, from about 100° to about 150°, from about 105° to about 145°, from about 110° to about 140°, at least about 100°, at least about 110°, or at least about 120°, including all ranges and subranges therebetween. In examples, the dynamic contact angles, i.e., the contact angles which occurs in the course of wetting (advancing angle) or de-wetting (receding angle) of a surface for the biointerface/bioactive releasing membrane has an advancing contact angle of about 100° to about 150°. In another example, the dynamic contact angles, i.e., the contact angles which occurs in the course of wetting (advancing angle) or de-wetting (receding angle) of a surface for the biointerface/bioactive releasing membrane has an advancing contact angle of about 105° to about 130°, or 110° to about 120°, including all ranges and subranges therebetween. In yet another example, the dynamic contact angles, i.e., the contact angles which occurs in the course of wetting (advancing angle) or de-wetting (receding angle) of a surface for the biointerface/bioactive releasing membrane has a receding contact angle of about 40° to about 80°. In another example, the dynamic contact angles, i.e., the contact angles which occurs in the course of wetting (advancing angle) or de-wetting (receding angle) of a surface for the biointerface/bioactive releasing membrane has a receding contact angle of about 45° to about 75°. In yet another example, the dynamic contact angles, i.e., the contact angles which occurs in the course of wetting (advancing angle) or de-wetting (receding angle) of a surface for the biointerface/bioactive releasing membrane has a receding contact angle of about 50° to about 70°. In some examples, dynamic contact angle measurements and surface roughness (correlated to contact angle hysteresis, which arises from the chemical and topographical heterogeneity of the surface, solution impurities absorbing on the surface, or swelling, rearrangement, or alteration of the surface by the solvent) on the bioactive releasing membrane after placement on the analyte sensor and after sterilization can be carried out using a Sigma 701 force tensiometer and performing one or more of advancing contact angle measurements, receding contact angle measurements, hysteresis measurements, and combinations thereof. The force tensiometer measures the mass affecting to the balance and calculates and automatically subtracts the effects of the buoyancy force and the weight of the probe such that the only remaining force being measured by the balance is the wetting force.

In examples, the bioactive releasing membrane 70 has a hard segment weight percent content of between about 20-60%, 30-50%, or 35-45% so as to achieve a hardness of 70A-55D durometer. In another example, the bioactive releasing membrane 70 has a hard segment weight percent content of between about 20-60%, 30-50%, or 35-45% so as to achieve a target modulus. In one example, the durometer hardness and/or modulus of the bioactive releasing membrane 70 is provided by a single copolymer or blends of copolymers.

In one example, the bioactive releasing membrane 70 comprises a soft segment-hard segment copolymer comprising less than 70 weight percent of soft segment, not including zero weight percent. In one example, the releasing membrane comprises a soft segment-hard segment copolymer comprising a soft segment-hard segment polyurethane or polyurethane urea copolymer comprising less than 70 weight percent of soft segment, not including zero weight percent.

In one example, the bioactive releasing membrane comprises a soft segment-hard segment copolymer comprising a hydrophilic segment weight percent that is greater than the hydrophobic segment weight percent thereof. In examples, the releasing membrane comprises a soft segment-hard segment polyurethane or polyurethane urea copolymer comprising a hydrophilic segment weight percent of a soft segment-hard segment that is greater than the hydrophobic segment weight percent thereof.

In one example, the hydrophilic segment weight percent of the soft segment-hard segment copolymer is less than the hydrophobic segment weight percent thereof. In one example, the hydrophilic segment weight percent of the soft segment-hard segment polyurethane or polyurethane urea copolymer is less than the hydrophobic segment weight percent thereof.

In one example, the bioactive releasing membrane comprises a soft segment-hard segment copolymer that is blends of different soft segment-hard segment copolymers. In one example, the bioactive releasing membrane comprises a soft segment-hard segment polyurethane or polyurethane urea copolymer that is blends of different soft segment-hard segment copolymers.

In one example, the bioactive releasing membrane comprises a blend of different soft segment-hard segment copolymers that is a first soft segment-hard segment copolymer comprising a hydrophilic segment, not including zero weight percent, and a hydrophobic segment, including zero weight percent, blended with another second soft segment-hard segment copolymer comprising a hydrophilic segment weight percent greater than a hydrophobic segment weight percent. In one example, the bioactive releasing membrane comprises a blend of different soft segment-hard segment polyurethane or polyurethane urea copolymers that comprise a first soft segment-hard segment copolymer comprising a hydrophilic segment, not including zero weight percent, and a hydrophobic segment, including zero weight percent, blended with another soft segment-hard segment polyurethane or polyurethane urea copolymer comprising a hydrophilic segment weight percent greater than a hydrophobic segment weight percent.

In one example, the bioactive releasing membrane comprises a soft segment-hard segment copolymer comprising a hydrophilic segment, not including zero weight percent, and a hydrophobic segment, including zero weight percent, blended with another soft segment-hard segment copolymer comprising a hydrophilic segment weight percent less than a hydrophobic segment weight percent. In one example, the bioactive releasing membrane comprises a soft segment-hard segment polyurethane or polyurethane urea copolymer comprising a hydrophilic segment, not including zero weight percent, and a hydrophobic segment, including zero weight percent, blended with another soft segment-hard segment polyurethane or polyurethane urea copolymer comprising a hydrophilic segment weight percent less than a hydrophobic segment weight percent.

In one example, the bioactive releasing membrane comprises a soft segment-hard segment copolymer and a soft segment-hard segment copolymer, each comprising less than 70 weight percent of soft segment, not including zero weight percent, and each comprising a hydrophilic segment, not including zero weight percent, and a hydrophobic segment, including zero weight percent. In one example, the bioactive releasing membrane comprises a soft segment-hard segment polyurethane or polyurethane urea copolymer and another, different, soft segment-hard segment polyurethane or polyurethane urea copolymer, each comprising less than 70 weight percent of soft segment, not including zero weight percent, and each comprising a hydrophilic segment, not including zero weight percent, and a hydrophobic segment, including zero weight percent.

In one example, the bioactive releasing membrane comprises a soft segment-hard segment copolymer blended with a hydrophobic polymer and/or a hydrophilic polymer. In one example, the bioactive releasing membrane comprises a soft segment-hard segment polyurethane or polyurethane urea copolymer blended with a hydrophobic polymer and/or a hydrophilic polymer.

In one example, the bioactive releasing membrane 70 is substantially impervious to analyte transport there through. In another example, the bioactive releasing membrane 70 is less permeable to the analyte than the interference membrane 44 of the sensing membrane 32. In such examples, the bioactive releasing membrane 70 is deposited on portions of the sensor adjacent to but not covering the electrochemically active portion of the sensor.

In one example, the bioactive releasing membrane 70 is loaded with bioactive agent prior to depositing on the sensor 34 and/or sensing membrane 32. In one example, the bioactive agent is dissolved in one or more solvents that are miscible with the bioactive releasing membrane 70. Mild heating can be used to facilitate dissolution, distribution, or dispersing of the bioactive agent in the bioactive releasing membrane 70. Suitable solvents include THF, alcohols, ketones, ethers, acetates, NMP, methylene chloride, heptane, hexane, and combinations thereof.

In one example, the bioactive releasing membrane 70 is deposited onto at least a portion of the sensing membrane 32. In another example, the bioactive releasing membrane 70 is deposited adjacent to but not directly on sensing membrane 32. In examples, the bioactive releasing membrane is deposited so as to provide a membrane thickness of from about 0.05 micron or more to about 50 microns or less, including all ranges and subranges therebetween. In another example, the bioactive releasing membrane is deposited so as to provide a membrane thickness of from about 0.5 to 50 microns, 1 to 50 microns, 2 to 50 microns, 3 to 50 microns, 4 to 50 microns, 5 to 50 microns, 6 to 50 microns, 7 to 50 microns, 8 to 50 microns, 9 to 50 microns, 10 to 50 microns, 10 to 40 microns, 10 to 30 microns, 10, 11, 12, 13, 14, 15, 16, 17, 18, 19, 20, 21, 22, 23, 24, 25, 26, 27, 28, 29 or 30 microns, including all ranges and subranges therebetween.

In examples, the bioactive releasing membrane 70 is deposited onto the enzyme domain by spray coating, brush coating, pad printing, or dip coating. In certain examples, the bioactive releasing membrane 70 is deposited using spray coating and/or dip coating. In examples, the bioactive releasing membrane 70 is deposited on the sensing membrane 32 by pad-printing a mixture of from about 1 wt. % to about 80 wt. % polymer/drug combination and from about 20 wt. % to about 99 wt. % solvent, including all ranges and subranges therebetween.

In contacting a solution of bioactive releasing membrane 72, including a solvent, onto the sensing membrane, it is desirable to mitigate or substantially reduce any contact with enzyme of any solvent in the pad printing mixture that can deactivate the underlying enzyme of the enzyme domain. Tetrahydrofuran (THF) is one solvent, alone or in combination with one or more alcohols, that minimally or negligibly affects the enzyme of the enzyme domain upon spraying. Other solvents can also be suitable for use, as is appreciated by one skilled in the art.

In examples, the bioactive releasing membrane 70 is deposited on the sensing membrane 32 by spray-coating a solution of from about 1 wt. % to about 50 wt. % polymer and from about 50 wt. % to about 99 wt. % solvent, including all ranges and subranges therebetween. In spraying a solution of bioactive releasing membrane 72, including a solvent, onto the sensing membrane, it is desirable to mitigate or substantially reduce any contact with enzyme of any solvent in the spray solution that can deactivate the underlying enzyme of the enzyme domain. Tetrahydrofuran (THF) is one solvent, alone or in combination with one or more alcohols, that minimally or negligibly affects the enzyme of the enzyme domain upon spraying. Other solvents can also be suitable for use, as is appreciated by one skilled in the art.

### Bioactive Releasing Membrane/Layer Compositions-Bioactive Agent Release Profiles

The present disclosure provides for control of release, or for providing a release profile, of the bioactive agent from the bioactive releasing membrane. By way of example, an exemplary bioactive agent/bioactive releasing membrane system is used, e.g., dexamethasone and/or dexamethasone acetate salt/soft segment-hard segment polyurethane urea copolymer or blends, however, other combinations of bioactive agents and bioactive releasing membranes are envisioned.

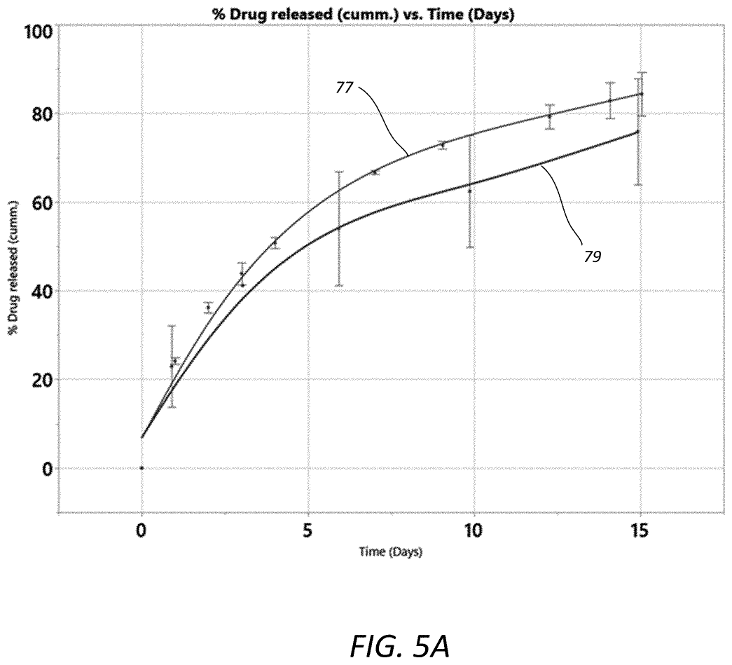

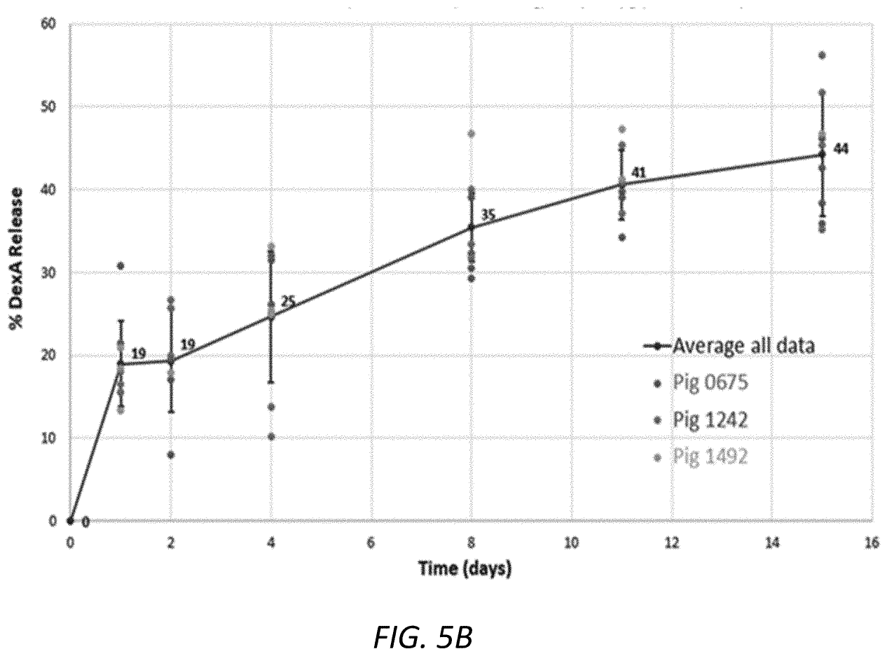

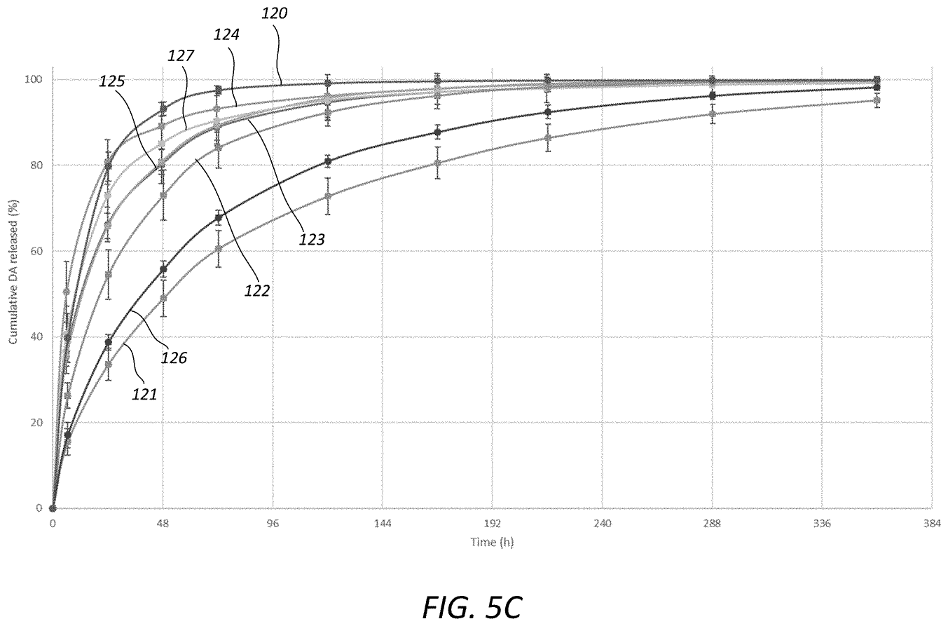

With reference to FIG. 5A, an exemplary in vitro bioactive release profiles for dexamethasone acetate is shown using exemplary bioactive releasing membrane 70. The percent cumulative release of dexamethasone acetate can be determined using HPLC, for example using a Phenomenex Kinetex 5μ EVO C18 100 Å, 50×3.0 mm column held at 25° C. with a 254 nm UV detector and an elution gradient of A: Water with 0.1% formic acid/B: Acetonitrile with 0.1% formic acid (vol/vol), where the gradient from time 0 to 2 minutes is 90% A/10% B; from 2-5 minutes is 10% A/90% B; and from 5 minutes is 90% A/10% B. Dexamethasone acetate and dexamethasone HPLC standards are prepared at concentrations of about 0.1-20 ug/mL.

FIG. 5A shows a correlation between in vitro 77 and in vivo 79 release of dexamethasone acetate in the presently disclosed bioactive releasing membrane 70 over a 15 day period that demonstrates the viability of in vitro data for approximating in vivo data of the presently disclosed system.

With reference to FIG. 5B, experimental data of a release rate of a bioactive agent (dexamethasone acetate) from a bioactive releasing membrane 70 initially or during the first time period being greater than the release rate of the bioactive agent from the bioactive releasing membrane initially or during the second time period and the release rate of the bioactive agent from the bioactive releasing membrane initially or during the second time period is greater than the release rate of the bioactive agent from the bioactive releasing membrane initially or during the third time period is shown.

Thus, FIG. 5B depicts the exemplary in vitro bioactive release profile of FIG. 5A is shown having a first release rate indicated as corresponding to a time period associated with sensor insertion and extending approximately 2 days or more (e.g., a bolus), followed by a second release rate indicated as corresponding to a second time period associated with a time approximately beginning at about 2 days and extending upwards of 15 days after sensor insertion e.g., (an amount within the therapeutic range, or a “sustained therapeutic amount”). A release of an amount of less than the therapeutic amount, e.g., a non-therapeutic amount, during a time approximately 18 days or more after sensor insertion and continuing until the end-of-life of the sensor results (data not shown). As can be seen by the graphical data of FIG. 5B, the first release rate corresponding to a bolus release of approximately 50% of the initial loading of dexamethasone acetate over approximately a two day period, followed by a second release rate corresponding to a release of approximately 40% of the initial loading of dexamethasone acetate over a time span of about 13 days. A third release rate corresponding to a release of the remaining amount of dexamethasone acetate (approximately 10%) over a time span of 16-35 days follows.

Thus, with an initial loading of 50-100 μg dexamethasone acetate (DexAc)/sensor, for example, where a therapeutically effective amount or more of release per day is targeted, the presently disclosed bioactive releasing membrane 70 can provide a bolus therapeutic release of an amount of DexAc immediately upon insertion (approximately 3-20 μg/sensor/day, 4-18 ag/sensor/day, 5-16 ag/sensor/day, 6-14 ag/sensor/day) and for a period thereafter, followed by an extended therapeutic release of an amount of DexAc (approximately 0.5-10 μg/sensor/day, 0.6-nine μg/sensor/day, 0.4-7 μg/sensor/day, 0.5-8 μg/sensor/day), followed by an extended non-therapeutic release of an amount of DexAc (approximately less than 0.5 μg/sensor/day) until end-of-life of the sensor.

With reference to FIG. 5C, initial and sustained bioactive release rates with different bioactive releasing membranes are presented. As shown, polyurethane polymer membrane examples with varying amounts of polysiloxane component in the soft segment, ranging from about 10 wt. % to about 40 wt. %, each of the examples having a hard segment of between 40-55 wt. % demonstrated unique release rates of dexamethasone acetate over an initial 10-48 hr time period as well as different total release amounts over an extended time period of up to 15 days as summarized as follows: Sample 120-10 wt. % polysiloxane: 50 wt. % hard segment Sample 121-22 wt. % polysiloxane: 55 wt. % hard segment; Sample 122-25 wt. % polysiloxane: 50 wt. % hard segment; Sample 123-27 wt. % polysiloxane: 45 wt. % hard segment; Sample 124-30 wt. % polysiloxane: 40 wt. % hard segment; Sample 125-30 wt. % polysiloxane: 45 wt. % hard segment; Sample 126-30 wt. % polysiloxane: 50 wt. % hard segment; Sample 127-40 wt. % polysiloxane: 40 wt. % hard segment. For example, a 10 wt. % polysiloxane-containing membrane provided a rapid initial release rate and a sustained high total release rate in contrast to a 35 wt. % polysiloxane-containing membrane providing a more linear-like release rate and a sustained low total release rate. The data of FIG. 5C further demonstrates the effect of the hard segment wt. % in combination with the polysiloxane-containing membrane to tailor the bioactive release rate initially and for sustained time durations. Thus, a desired or targeted bioactive release profile commensurate with a bioactive therapeutic regimen is obtainable with modification of the chemical make-up of the bioactive releasing membrane 70.

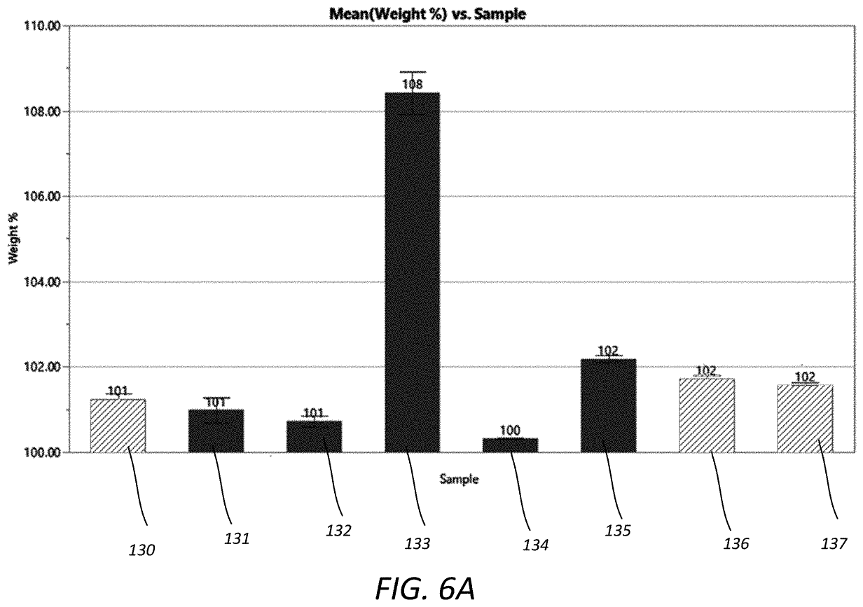

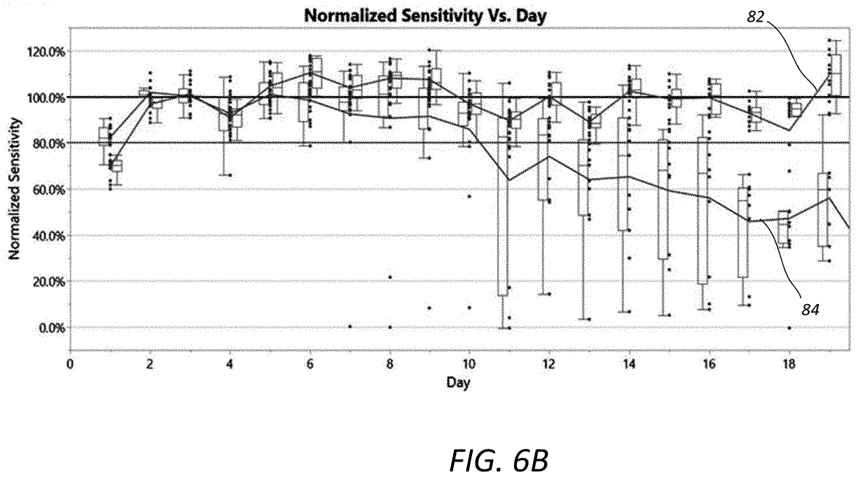

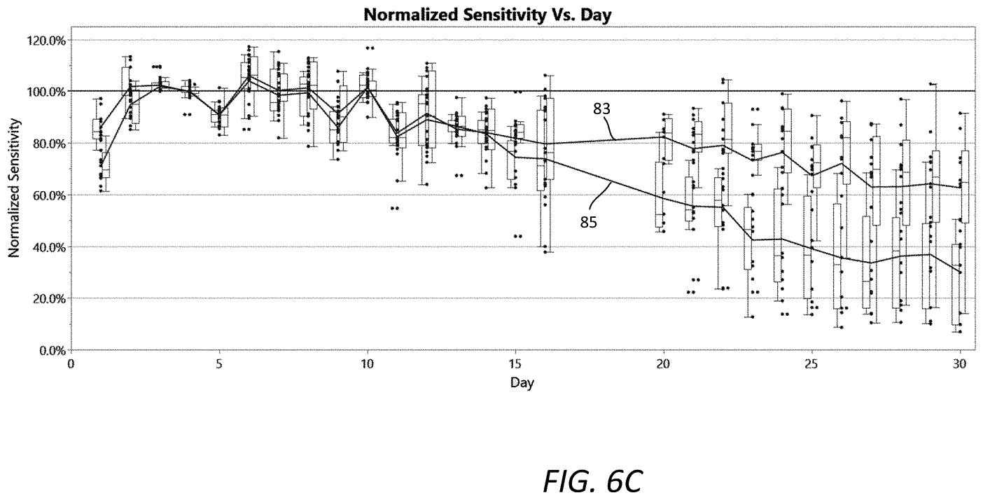

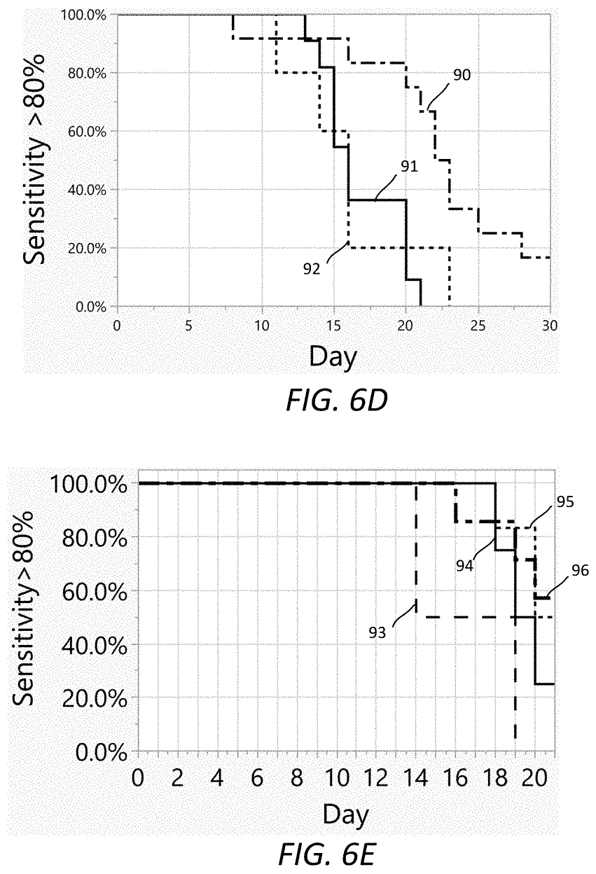

With reference to FIG. 6A the effect of bioactive releasing membrane 70 chemistry on bioactive release is correlated with water uptake of the membrane. In one example, at least a portion of the bioactive releasing membrane 70 (e.g., a hard segment) has an Hilderbrand solubility parameter closer to the releasable bioactive agent than another portion of the bioactive releasing membrane (e.g. a soft segment). For example, the bioactive releasing membrane 70 can comprise a hydrophobic soft segment, at least one hydrophilic soft segment, and a hard segment comprising urethane groups, urea groups, or a combination of urethane groups and urea groups, where the hard segment thereof has an Hilderbrand solubility parameter closer to the releasable bioactive agent than either of the soft segment portions. FIG. 6A shows various samples of bioactive releasing membrane 70 with different hard segment portions (and different wt. % ranges) and hydrophobic soft segment and hydrophilic soft segment portions with varying wt. % ranges. Thus, Samples 130, 136 and 137 comprising polyurethane block polymers with 40-60 wt. % hard segment (e.g., cyclic isophorone diisocynate (IPDI)), 10-30 wt. % hydrophobic soft segment portion (e.g., polysiloxane) and 20-50 wt. % hydrophilic soft segment portion (e.g., polyalkylether) displayed a desirable release rate for a selected bioactive (e.g., dexamethasone acetate). In contrast, Samples 131, 132, 133, 134 and 135 comprising polyurethane block polymers with 40-60 wt. % hard segment (e.g., linear 1,6-hexamethylene diisocynate (HDI)), 10-30 wt. % hydrophobic soft segment portion (e.g., polysiloxane) and 0-50 wt. % hydrophilic soft segment portion (e.g., polyalkylether) displayed a rapid release for a selected bioactive (e.g., dexamethasone acetate). This data demonstrates a correlation of the water uptake of a bioactive releasing layer, e.g., the hard segment solubility being similar to the releasable bioactive, to that of the bioactive can be employed to tailor the release rate/profile of a bioactive.

In examples, the bioactive releasing membrane 70 chemistry comprises a soft segment and a hard segment comprising urethane groups, urea groups, or a combination of urethane groups and urea groups. The soft segment is two or more different polymer segments. The soft segment comprises a hydrophobic block and a hydrophilic block. The soft segment comprises polysiloxane, polyalkylether, polyalkylester, polyalkylcarbonate, polycarbonate, or polysiloxane-polyalkylether segmented blocks.

In examples, the bioactive releasing membrane 70 chemistry further comprises a chain extender. The chain extender comprises a diol, a diamine, a silicon-hydride, or a multifunctional epoxide.

In examples, the bioactive releasing membrane 70 chemistry is a polyurethane urea.

In examples, the bioactive releasing membrane 70 chemistry comprises about 10-30 wt. % polysiloxane and about 10-30 wt. % polyalkylether, 40-60% wt. % hard segment comprising urethane groups, urea groups, or a combination of urethane groups and urea groups, and any remainder wt. % being chain extender, based on a total weight of the bioactive releasing membrane.

In examples, the bioactive releasing membrane 70 chemistry comprises about 20-30 wt. % polysiloxane, about 20-30 wt. % polyalkylether, and about 40-60 wt. % hard segment, and any remainder wt. % being chain extender, based on a total weight of the bioactive releasing membrane.

In examples, the bioactive releasing membrane 70 chemistry comprises a soft segment comprising the about 10-30 wt. % polysiloxane, the about 10-30 wt. % polyalkylether, and the about 0-10 wt. % chain extender, based on a total weight of the bioactive releasing membrane.

In examples, the polyalkylether is represented by repeating units of formula (I): —(R5-O)—; where R5 is a linear or branched alkyl group of 2 to 6 carbons.

With reference to FIG. 6B, a study in a living host sensitivity data is presented of an exemplary experimental sensor 82 comprising the presently disclosed bioactive releasing membrane 70 with an effective amount of dexamethasone acetate (DexAc) (e.g., approximately 40-50 weight percent loading: bioactive releasing membrane) compared with a control sensor 84 having membrane 70 without DexAc over 15 days. As shown, the experimental sensor 82 provided consistent normalized sensitivity sustainability over the 15 days post insertion while the control sensor 84 showed a decrease in normalized sensitivity after approximately 10 days post insertion.

With reference to FIG. 6C, a study in a living host sensitivity data is presented of an exemplary experimental sensor 83 comprising the presently disclosed bioactive releasing membrane 70 with an effective amount of dexamethasone acetate (DexAc) (e.g., approximately 40-50 weight percent loading: bioactive releasing membrane) compared with a control sensor 85 without DexAc at 30 days. As shown, the experimental sensor 83 provided improved normalized sensitivity sustainability above 60% over the 30 days post insertion while the control sensor 84 showed a decrease below 60% in normalized sensitivity after approximately 20 days post insertion.

In some embodiments, sensitivity loss may be indicative of end of life. Sensitivity loss may occur towards the sensor end of life due to physiological wound healing and foreign body mechanisms around the sensor or other mechanisms including reference electrode capacity, enzyme depletion, membrane changes, or the like.

In some embodiments, sensor sensitivity may be computed in using an analysis of uncalibrated sensor data (e.g., raw or filtered). In examples, a slow moving average or median of raw count starts showing negative trends, the sensor may be losing sensitivity. Loss of sensitivity may be computed by calculating a short term (e.g. ˜6-8 hours) average (or median) of the sensor output and normalizing it by the expected longer term (48 hours) average sensor sensitivity. If the ratio of short term to long term sensitivity is smaller than 70%, there may be a risk of sensor losing sensitivity. Loss of sensitivity may be translated into an end of life risk factor value, for example a value of about 1 until the ratio is about 70%, reducing to 0.5 at 50% and <0.1 at 25%.

In some embodiments, sensor sensitivity may be computed by comparing sensor data (e.g., calibrated sensor data) with reference blood glucose. For example, calibration algorithms adjust the glucose estimates based on the systematic bias between sensor and a reference blood glucose. End of life algorithms may use this bias, called error at calibration or downward drift, to quantify or qualify end of life symptoms. The error at calibration may be normalized to account for irregular calibration times and smoothed to give more weight to recent data (e.g., moving average or exponential smoothing). In some embodiments, end of life risk factor value is determined based on the resulting smoothed error at calibration. In such embodiments, end of life risk factor value is 1 for all values of error at calibration>−0.3, and reduces to 0.5 at error at calibration=−0.4, and to <0.1 for error at calibration=−0.6. In some examples, one more of a downward drift in sensor sensitivity over time, an amount of non-symmetrical, nonstationary noise, and a duration of noise can be employed, for example, as disclosed in co-assigned U.S. Pat. Pub. No. 2021/0209497, which is incorporated herein by reference.

In some embodiments, sensor sensitivity may be computed in using an analysis of uncalibrated sensor data (e.g., raw or filtered). In examples, a slow moving average or median of raw count starts showing negative trends, the sensor may be losing sensitivity. Loss of sensitivity may be computed by calculating a short term (e.g. ˜6-8 hours) average (or median) of the sensor output and normalizing it by the expected longer term (48 hours) average sensor sensitivity. If the ratio of short term to long term sensitivity is smaller than 70%, there may be a risk of sensor losing sensitivity. Loss of sensitivity may be translated into an end of life risk factor value, for example a value of about 1 until the ratio is about 70%, reducing to 0.5% at 50% and <0.1% at 25%.

With reference to FIG. 6C, a survival plot of continuous analyte sensors 90 (with the bioactive releasing membrane 70) verses controls 91 (no membrane) and sensors 92 (with membrane but no bioactive present). As shown, the sensors with the bioactive releasing membrane 70 outperformed, by at least 5 days, the controls and sensors with just the membrane, where sensitivity of less than 80% is indicative of eminent end of life (EOL).

With reference to survival plot FIG. 6D, improvement to retention of sensitivity was demonstrated with modification to the bioactive releasing membrane chemistry, for example, adjusting the weight percent of hard segment, soft segment, weight percent of hydrophobic portion of the soft segment, etc. so as to alter the release rate of the bioactive from the bioactive releasing membrane 70, generally characterized as a fast release, medium release, or slow release of bioactive, including any bolus release or absence of bolus release. Thus, FIG. 6D shows a slow release rate membrane 93 of bioactive (exemplified by dexamethasone acetate), having less than 80% of sensitivity retention after 14 days, control 94 (no membrane) having less than 80% of sensitivity retention after 18 days, medium release rate membrane 95 having less than 80% of sensitivity retention after 19 days, and fast release rate membrane 96 having less than 80% of sensitivity retention after 20 days.

With reference to FIG. 7A, a study in a living host of mean absolute noise data is presented of an exemplary experimental sensor 86 comprising the presently disclosed bioactive releasing membrane 70 with an effective amount of dexamethasone acetate (DexAc) (e.g., approximately 40-50 weight percent loading: bioactive releasing membrane) compared with a control sensor 84 without DexAc over 22 days and a comparative sensor 87 with bioactive releasing membrane 70 without dexamethasone acetate. As shown, the experimental sensor 86 provided relatively consistent mean absolute noise sustainability over the 22 days post insertion while the control sensor 88 and comparative sensor 87 showed an increase in mean absolute noise after approximately 8-10 days post insertion. This data exemplifies the ability of the presently disclosed bioactive releasing membrane with bioactive agent combination minimizes the increase of noise of an implantable sensor over an extended time period.

With reference to FIG. 7B, a survival plot of continuous analyte sensors 90 (with the bioactive releasing membrane 70) verses controls 91 (no membrane) and sensors 92 (with membrane but no bioactive present) are presented. As shown, the sensors with the bioactive releasing membrane 70 outperformed, by at least 10 days, the controls and sensors with just the membrane, where noise of less than 80% is indicative of eminent end of life (EOL).

With reference to survival plot FIG. 7C, improvement to minimizing increase in noise was demonstrated with modification to the bioactive releasing membrane chemistry, for example, adjusting the weight percent of hard segment, soft segment, weight percent of hydrophobic portion of the soft segment, etc. so as to alter the release rate of the bioactive from the bioactive releasing membrane 70, generally characterized as a fast release, medium release, or slow release of bioactive, including any bolus release or absence of bolus release as previously described in FIG. 6D. Thus, FIG. 7C shows a slow release rate membrane 93 of bioactive (exemplified by dexamethasone acetate), having more than 80% of noise increase after 5 days, control 94 (no membrane) having more than 80% of noise increase after 4 days, medium release rate membrane 95 having more than 80% of noise increase after 8 days, and fast release rate membrane 96 having more than 80% of noise increase after 8 days. This data exemplifies the ability of the presently disclosed bioactive releasing membrane with bioactive agent combination minimizes the increase of noise of an implantable sensor over an extended time period relative to a control or non-bioactive membrane.

Additional experiments were carried out using dexamethasone salts in different bioactive releasing membrane combinations. For example dexamethasone sodium phosphate in a water-soluble cellulosic based polymer provided a bolus release profile. Dexamethasone phosphate incorporated in a biointerface polymer membrane as disclosed herein provided about 2 days sustained release. Dexamethasone acetate in a hard-soft segmented polyurethane urea copolymer with zero weight percent of hydrophobic soft segment provided about 5 days sustained release. Dexamethasone acetate in a hard-soft segmented polyurethane urea copolymer with approximately equal weight percentages hydrophobic/hydrophilic segments, provided approximately 15 days sustained release. Dexamethasone acetate in a hard-soft segmented polyurethane urea copolymer with a weight percent of hydrophobic soft segment greater than the weight percent of hydrophilic soft segment provided more than 15 days of slow, sustained release. Dexamethasone acetate in a cellulose polymer, provided more than 15 days of slow, sustained (continuous or semicontinuous) release. Using combinations of the aforementioned bioactive releasing membranes the release rate and/or release profile of the bioactive agents can be specifically tailored to the specific sensor and its intended end-of-life while providing sustained sensitivity and low noise performance.

This data exemplifies the ability of the presently disclosed bioactive releasing membrane/bioactive agent combination minimize decay/decrease of sensitivity of an implantable sensor over an extended time period. The presently disclosed bioactive releasing membrane/bioactive agent combination can be configured for other sensor platforms besides electrochemical based sensor systems such as optical based sensor systems, as well as other medical devices intended for extended implantation that need to be subsequently removed from the subject.

As shown in FIG. 3H, the continuous analyte sensing device 100 includes an analyte sensor having an insertable portion 102 operably coupled to a non-insertable portion 104, with the continuous analyte sensing device 100 being configured to deploy the insertable portion 102. The insertable portion 102 has at least one of an insertable surface are and an insertable volume. At least one sensing domain 112 is at least partially positioned about the insertable portion 102 (and, as such, the insertable surface area and/or the insertable volume). The insertable portion 102 also includes a bioactive releasing membrane 70 formed over the insertable portion 102.

In examples, the insertable portion 102 has a length of about 1 mm to about 20 mm, including all ranges and subranges therebetween. In another example, the insertable portion 102 has a length of about 2 mm to about 14 mm. In a further example, the insertable portion 102 has a length of about 4 mm to about 12 mm. For example, the insertable portion 102 has a length of at least about any of the following: 1, 2, 3, 4, 5, 6, 7, 8, 9, 10, 11, 12, 13, 14, 15, 16, 17, 18, and 19 mm and/or at most about 20, 19, 18, 17, 16, 15, 4, 13, 12, 11, 10, 9, 8, 7, 6, 5, 4, 3, and 2 mm (e.g., about 1-15 mm, about 5-18 mm, etc.).

As shown in FIG. 3I, the insertable portion comprises a bioactive releasing membrane 70. In one example, bioactive releasing membrane 70 includes at least one polymer layer disposed over a portion of the insertable portion 102 (and, as a result, a portion of the insertable surface area and/or insertable volume). The bioactive releasing membrane 70 includes at least one bioactive agent 110 dispersed in the bioactive releasing membrane. In one example, the bioactive releasing membrane 70 is configured to associate with and/or release at least one bioactive agent 110. The at least one bioactive agent 110 can be configured to be non-releasable from the bioactive releasing membrane and modify tissue response of a subject. The at least one bioactive agent 110 can independently be configured to be non-releasable in some form as well as releasable from the bioactive releasing membrane and modify tissue response of a subject.

The at least one polymer layer of the bioactive releasing membrane 70 can include any suitable polymeric materials discussed previously herein. In examples, the at least one polymer layer of the bioactive releasing membrane 70 comprises one or more epoxides, polyolefins, polysiloxanes, polyamide, polystyrene, polyacrylate, polyethers, polyvinyl pyridines, polyvinyl-co-polystyrene, polyvinylimidazoles, polyesters, polyalkylesters, polyalkylcarbonates, polycarbonates, polyurethane, polyurethaneurea, polyethylene vinyl acetate (EVA), polyvinyl alcohol, and copolymers or blends thereof. In another example, the at least one polymer layer of the bioactive releasing membrane 70 comprises one or more zwitterionic repeating units associated with the at least one bioactive agent, the at least one bioactive agent configured to be released from the one or more zwitterionic repeating units to modify tissue response of a subject.

In one aspect, alone or in combination with any one of the previous aspects, the bioactive releasing membrane comprises a polyethylene oxide segment.

In one aspect, alone or in combination with any one of the previous aspects, the polyethylene oxide segment is from about 5 wt. % to about 60 wt. %, including all ranges and subranges therebetween, based on the total weight of the bioactive releasing membrane.

In one aspect, alone or in combination with any one of the previous aspects, a base polymer of the bioactive releasing membrane has an average molecular weight of from about 10 kDa to about 500 kDa, including all ranges and subranges therebetween.

In one aspect, alone or in combination with any one of the previous aspects, the bioactive releasing membrane has a polydispersity index of from 1 to about 10, including all ranges and subranges therebetween.

In one aspect, alone or in combination with any one of the previous aspects, the bioactive releasing membrane has a contact angle of from about 90° to about 160°, including all ranges and subranges therebetween.

The at least one bioactive agent 110 includes any suitable bioactive agent discussed previously herein. In examples, the at least one bioactive agent 110 comprises an anti-inflammatory compound or a tissue response modifier. For instance, In examples, the at least one bioactive agent 110 comprises at least one of dexamethasone, a dexamethasone salt, a dexamethasone derivative, dexamethasone acetate, or any combination thereof.

In examples, at least one bioactive agent 110 is dispersed in the at least one polymer layer of the bioactive releasing membrane 70 (and, as a result, the polymer layer volume) at a drug/polymer weight/weight ratio of from about 0.1 to about 2, including all ranges and subranges therebetween. In another example, at least one bioactive agent 110 is dispersed in the at least one polymer layer of the bioactive releasing membrane 70 (and, as a result, the polymer layer volume) at a drug/polymer weight/weight ratio of from about 0.1 to about 0.3. In a further example, at least one bioactive agent 110 is dispersed in the at least one polymer layer of the bioactive releasing membrane 70 (and, as a result, the polymer layer volume) at a drug/polymer weight/weight ratio of about 0.3 to about 0.5. For example, the at least one bioactive agent 110 is dispersed in the at least one polymer layer of the bioactive releasing membrane 70 at a drug/polymer weight/weight ratio from at least about any of the following: 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, and 0.49 and/or at most about 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.19, 0.18, 0.17, 0.16, 0.15, 0.14, 0.13, 0.12, 0.11, 0.1, 0.09, 0.08, 0.07, 0.06, 0.05, 0.04, 0.03, and 0.02 (e.g., about 0.19-0.49, about 0.01-0.3, etc.). In a further example, at least one bioactive agent is dispersed in the polymer layer volume at a drug/polymer weight/volume ratio of from about 0.1:2 μg/mm³ to about 0.2:1 μg/mm³, including all ranges and subranges therebetween.

In another example, at least one bioactive agent is dispersed in the bioactive releasing membrane 70 volume at a drug/polymerweight/volume ratio of from about 0.2 μg/mm³ to about 1 μg/mm³, including all ranges and subranges therebetween. In another example, at least one bioactive agent is dispersed in the polymer layer volume at a drug/polymer weight/volume ratio of about 1:10 μg/mm³ to about 2:1 μg/mm³. For example, the at least one bioactive agent dispersed in the polymer layer volume at a drug/polymer weight/volume ratio of from at least about any of the following: 1:10, 2:10 (i.e. 1:5), 3:10, 4:10 (i.e. 2:5), 5:10 (i.e. 1:2), 6:10 (i.e. 3:5), 7:10, 8:10 (i.e. 4:5), 9:10, 10:10 (i.e. 1:1), 11:10, 12:10 (i.e. 6:5), 13:10, 14:10 (i.e. 7:5), 15:10 (i.e. 3:2), 16:10 (i.e. 8:5), 17:10, 18:10 (i.e. 9:5), and 19:10 μg/mm³ and/or at most about 20:10 (i.e. 2:1), 19:10, 18:10 (i.e. 9:5), 17:10, 16:10 (i.e. 8:5), 15:10 (i.e. 3:2), 14:10 (i.e. 7:5), 13:10, 12:10 (6:5), 11:10, 10:10 (i.e. 1:1), 9:10, 8:10 (i.e. 4:5), 7:10, 6:10 (i.e. 3:5), 5:10 (i.e. 1:2), 4:10 (i.e. 2:5), 3:10, and 2:10 (i.e. 1:5) μg/mm³ (e.g., about 1:10-19:10, about 7:10-17:10, etc.), including all ranges and subranges therebetween. Loading of the at least one bioactive agent 110 has been discussed in more detail previously herein.

In another example, and as shown in FIG. 3J, the insertable portion coating is bioactive releasing membrane 70 that may include at least one polymer layer, and the at least one bioactive agent 110 is included in the membrane 70. In this example, the bioactive releasing membrane 70 may be adjacent the sensing membrane 32, and include being adjacent to any interferent membrane/domain, resistance membrane/domain, biointerface membrane/domain, and electrode membrane/domain. The various chemistries for bioactive releasing membranes 70, their structure, bioactive loading, among other features as contemplated in this disclosure have been discussed previously herein.

As discussed previously herein, while some figures (e.g., FIGS. 31 and 3J) herein illustrate sensors that may have a coaxial core and a circular or elliptical cross-section, in other examples of sensor systems including biointerface/bioactive release layer(s), the sensor may be a substantially planar sensor, as shown in the cross-section for illustration purposes in FIG. 2H. For example, as shown in FIG. 2H, the continuous analyte sensing device can include a substantially planar substrate 142, as well as an interference domain 144, an enzyme domain 146, a resistance domain 148, and a biointerface/bioprotective domain 168 and/or a bioactive releasing domain 170 arranged in a substantially planar fashion around the planar or substantially planar substrate 142.

As shown in FIGS. 3K and 3L, in some examples, the bioactive releasing membrane 70 is spatially separated from the at least one sensing domain 112 by a distance such that the bioactive releasing membrane 70 is not overlapping with the at least one sensing domain 112 or otherwise interfering with a diffusional path of the one or more analytes or other substances associated with the generation of a measurable signal corresponding to the amount or presence of the one or more analytes. In this way, the bioactive releasing membrane 70 does not interfere with the sensing capabilities of the at least one sensing domain 112. In examples, the distance is from about 1 μm or less to about 250 μm, including all ranges and subranges therebetween. In another example, the distance is from about 1 μm or less to about 50 μm. In yet another example, the distance is from about 15 μm to about 200 μm. In yet another example, the distance is from about 50 μm to about 100 μm. For example, the distance is from at least about any of the following: 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, and 245 μm and/or at most about 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, 10, and 5 μm (e.g., about 5-220 μm, about 45-250, etc.).

In examples, and as illustrated in FIG. 3K, the bioactive releasing membrane 70 is disposed on the insertable portion surface (and, as a result, the insertable portion surface area) and not in contact with the at least one sensing domain 112. In some examples, the bioactive releasing membrane 70 extends up to the entire length of the insertable portion 102 other than regions in contact with the at least one sensing domain 112. In another example, as illustrated in FIG. 3L, the bioactive releasing membrane 70 is only disposed at the distal end 109 of the insertable portion 102 (e.g., the insertable portion 102 is not disposed on or in contact with the at least one sensing domain 112, the proximal end 107 of the insertable portion 102, the remainder of the insertable portion 102, and/or the like). As further shown in FIG. 3L, the insertable portion 102 has a distal end 109, and, in one example as shown, the distal end 109 is spatially separated from the at least one sensing domain 112.

As also shown in FIG. 3K, in one example the insertable portion 102 is discontinuous or segmented, e.g., spanning the sensing domain 112. In another example, and as illustrated in FIG. 3L, the insertable portion 102 is continuous. Any shape suitable for coating the insertable portion, as understood by a person of ordinary skill in the art, may be used either continuously and/or discontinuously (e.g., the insertable portion coating can be continuous, discontinuous, or semi-continuous). Exemplary coating shapes include, but are not limited to, one or more cylinders, circles, ovals, squares, rectangles, triangles, diamonds, teardrops, helices, spirals, lobed shapes (e.g., clover, flower, butterfly, heart, etc.), and/or the like.

The insertable portion 102 has an insertable surface area and/or insertable volume, and the bioactive releasing membrane 70 has a surface are and/or a volume. In one example, the bioactive releasing membrane 70 surface area is less than or equal to the insertable surface area. In a further example, the bioactive releasing membrane 70 volume is the same or different from the insertable volume (e.g., less than or equal to the insertable volume).

With reference to FIGS. 3M, 3N, and 3O, sensors similar to those depicted in FIGS. 3K, 3L are presented showing the relationship of the bioactive releasing membrane 70 proximal to the distal tip of the sensor substrate, without covering the sensing membrane 32 (e.g., including the electrode, interference, resistance, biointerface membrane/domains) of the sensing domain 112. FIG. 3M shows the bioactive releasing membrane 70 about the distal tip without covering the singulation 29 of the wire or planar or substantially planar substrate. FIG. 3N shows an alternative construction with the bioactive releasing membrane 70 covering an end-cap 40 directly adjacent the singulation 29. FIG. 3O depicts another configuration where end-cap 40 is directly adjacent the singulation 29 and bioactive releasing membrane 70 is positioned about the distal tip of the insertable portion 102 without covering end-cap 40.

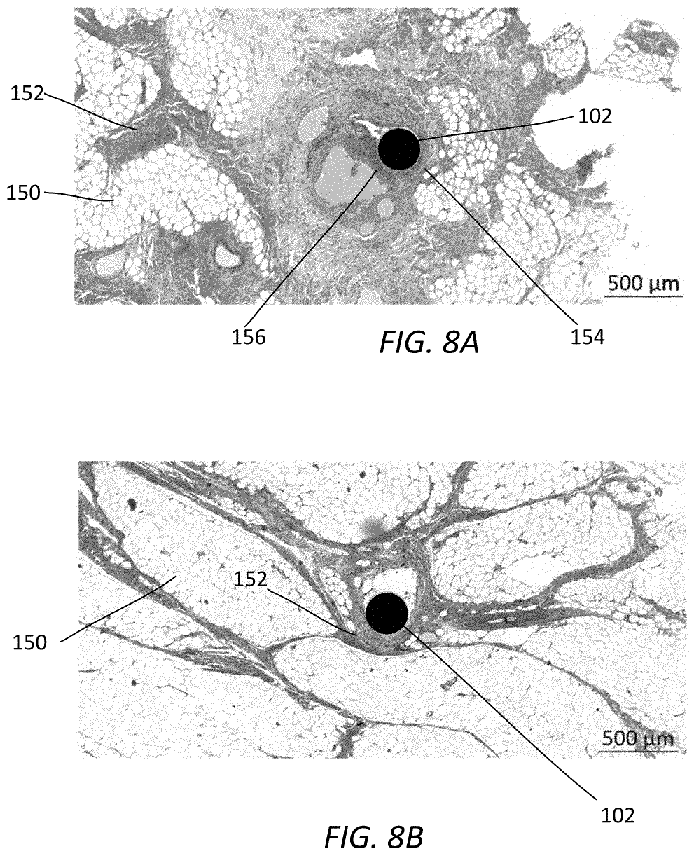

With reference to FIGS. 3P and 3Q, bioactive release of sensors with bioactive releasing membrane 70 located at the distal tip (e.g., “tip-coated” bioactive releasing membrane sensors) provide bioactive release profiles and equivalent improvement in sensitivity retention and noise reduction over extended use, e.g., 14 days, 21 days, 30 days, or more as other presently disclosed constructs described herein. Sensors with tip-coated bioactive releasing membranes with bioactive provide, among other advantages, ease of manufacturing, a reduction in total bioactive required, and targeted delivery of bioactive at the point of trauma, e.g., essentially immediate presentation of bioactive upon insertion of the sensor at the initial insertion site, delivery of API closest to where is need, i.e. sensing region, further physical protection from tip breaching, and/or delivery of API closest to the wound volume created by the space between the needle tip and the sensor tip. The natural surface tension of the tip coating forms a rounded surface that leads to protection of the tissues from puncture damage and the overall curved geometry is believed to help with biocompatibility. The tip-coated bioactive releasing membranes can be added to the sensors without vastly changing mechanical properties of the whole sensor. Manufacturing the sensors with tip-coated bioactive releasing membranes can be accomplished by dip coating, which can be a rapid and inexpensive manufacturing step. Examples of histology pictures of the insertable portion 102 of a sensor are shown in FIGS. 8A and 8B, where in FIG. 8A a microtome section of a stained histography depicting a subcutaneous section of tissue of a host after the insertable portion 102 of the sensor after an extended duration has resulted in a foreign body response of the immune system. Adipose tissue 150 and fibrous tissue 152 are depicted along with fibrotic encapsulation 154 and possibly cellular ingress 156. In contrast, FIG. 8B, depicts a microtome section of a stained histography of the insertable portion 102 of a sensor inserted in tissue of the host for an extended duration that included a bioactive releasing layer with a bioactive of the present disclosure. Adipose tissue 150 and fibrous tissue 152 are depicted without similar observable signs of fibrotic encapsulation or cellular ingress.

While not wishing to be bound by theory, it is believed that the tissue located at the end of the sensor suffers from the greatest degree of tissue trauma due to the sensor insertion. By providing the bioactive agent at the tip of the sensor, the foreign body response in the tissue proximate to the sensor tip reduced since the localized concentration of the bioactive agent is greatest in this surrounding environment. This also allows for less bioactive agent to be used within the bioactive releasing membrane 70. Any lag in time due to the transport of the bioactive agent through the tissue that would occur if the bioactive releasing membrane were placed proximally from the tip is minimized. Also, by coating the tip of the sensor with the bioactive releasing membrane 70, the overall insertion depth of the sensor and bioactive agent is reduced as compared to placing a mass of the bioactive agent near the sensor tip. The mass of the bioactive agent near the sensor tip could be inadvertently separated from the sensor tip pre-insertion or post-insertion resulting in a loss of therapeutic effect. In one example the bioactive releasing membrane 70 with bioactive is directly contacting and coating the sensing membrane present on the outer surface of the insertable tip portion of the sensor device. In one example the bioactive releasing membrane 70 with bioactive agent is only present on the outside surface of the sensor of the insertable portion. The outside surface can include any and all of the electrode domain, interference membrane, resistance membrane, and enzyme membranes previously disclosed and the bioactive releasing membrane 70 with bioactive agent can be more distal from the electrode surface than any of these domains or membranes. In one example the bioactive releasing membrane 70 with bioactive agent is most distal from the electrode surface than the electrode domain, interference membrane, resistance membrane, and enzyme membranes. In one example the diffusion adjustment membrane 73 is more distal from the diffusion bioactive releasing membrane 70 with bioactive agent. In one example the bioactive releasing membrane 70 with bioactive agent is present only on the outside surface of the sensor of the insertable portion directly adjacent the distal end 109 of the insertable portion 102, e.g., a tip-coated sensor, as shown in FIGS. 2E, 3H-3O.

In examples, the insertable surface area is from about 2 mm² to about 200 mm², including all ranges and subranges therebetween. In another example, the insertable surface area is from about 5 mm² to about 150 mm². In a further example, the insertable surface area is from about 25 mm² to about 100 mm². For example, the insertable surface area is from at least about any of the following: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, and 198 mm² and/or at most about 200, 198, 196, 194, 192, 190, 188, 186, 184, 182, 180, 178, 176, 174, 172, 170, 168, 166, 164, 162, 160, 158, 156, 154, 152, 150, 148, 146, 144, 142, 140, 138, 136, 134, 132, 130, 128, 126, 124, 122, 120, 118, 116, 114, 112, 110, 108, 106, 104, 102, 100, 98, 96, 94, 92, 90, 88, 86, 84, 82, 80, 78, 76, 74, 72, 70, 68, 66, 64, 62, 60, 58, 56, 54, 52, 50, 48, 46, 44, 42, 40, 38, 36, 34, 32, 30, 28, 26, 24, 22, 20, 18, 16, 14, 12, 10, 8, 6, and 4 mm² (e.g., about 30-172 mm², about 2-198 mm², etc.).

In examples, the insertable volume is from about 5 mm³ to about 500 mm³, including all ranges and subranges therebetween. In another example, the insertable volume is from about 10 mm³ to about 250 mm³. In a further example, the insertable volume is from about 25 mm³ to about 150 mm³. For example, the insertable volume is from at least about any of the following: 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, and 495 mm³ and/or at most about 500, 495, 490, 485, 480, 475, 470, 465, 460, 455, 450, 445, 440, 435, 430, 425, 420, 415, 410, 405, 400, 395, 390, 385, 380, 375, 370, 365, 360, 355, 350, 345, 340, 335, 330, 325, 320, 315, 310, 305, 300, 295, 290, 285, 280, 275, 270, 265, 260, 255, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, 30, 25, 20, 15, and 10 mm³ (e.g., about 20-420 mm³, about 10-500 mm³, etc.). The above ranges assume a maximum insertable portion coating thickness of about 5 mm.

In examples, a ratio of the polymer layer surface area to the insertable surface area is from about 0.1 to about 1, including all ranges and subranges therebetween. In another example, a ratio of the polymer layer surface area to the insertable surface area is from about 0.2 to about 0.8. In a further example, a ratio of the polymer layer surface area to the insertable surface area is from about 0.3 to about 0.7. For example, the ratio of the polymer layer surface area to the insertable surface area is from at least about any of the following: 0.1, 0.2, 0.3, 0.4, 0.5, 0.6, 0.7, 0.8, and 0.9 and/or at most about 1, 0.9, 0.8, 0.7, 0.6, 0.5, 0.4, 0.3, and 0.2 (e.g., about 0.2-0.9, about 0.5-0.8, etc.). The above ranges assume an active area of the sensor being at least 10% of the insertable portion.

In examples, a ratio of the polymer layer volume of the bioactive releasing membrane 70 to the insertable volume is from about 1:100 to about 20:100 (i.e. 1:5), including all ranges and subranges therebetween. In another example, a ratio of the polymer layer volume of the bioactive releasing membrane 70 to the insertable volume is from about 1:50 to about 15:75 (i.e. 1:5). In a further example, a ratio of the polymer layer volume to the insertable volume is from about 5:50 (i.e. 1:10) to about 10:80 (i.e. 1:8). For example, the ratio of the polymer layer volume to the insertable volume is from at least about any of the following: 1:100, 2:100 (i.e. 1:50), 3:100, 4:100 (i.e. 1:25), 5:100 (i.e. 1:20), 6:100 (i.e. 3:50), 7:100, 8:100 (i.e. 2:25), 9:100, 10:100 (i.e. 1:10), 11:100, 12:100 (i.e. 3:25), 13:100, 14:100 (i.e. 7:50), 15:100 (i.e. 3:20), 16:100 (i.e. 4:25), 17:100, 18:100 (i.e. 9:50), and 19:100 and/or at most about 20:100 (i.e. 1:5), 19:100, 18:100 (i.e. 9:50), 17:100, 16:100 (i.e. 4:25), 15:100 (i.e. 3:20), 14:100 (i.e. 7:50), 13:100, 12:100 (i.e. 3:25), 11:100, 10:100 (i.e. 1:10), 9:100, 8:100 (i.e. 2:25), 7:100, 6:100 (i.e. 3:50), 5:100 (i.e. 1:20), 4:100 (i.e. 1:25), 3:100, and 2:100 (i.e. 1:50) (e.g., about 7:100-19:100, about 3:100-17:100, etc.).

The continuous analyte sensing device described above is formed by providing a continuous analyte sensing device, and applying an insertable portion coating composition, which includes at least one polymer and at least one bioactive agent, to the insertable portion in order to provide the insertable portion coating described herein. In examples, the insertable portion coating composition as applied to the insertable portion has a viscosity of from about 10 cP to about 350 cP with or without bioactive loading. In another example, the insertable portion coating composition as applied to the insertable portion has a viscosity from about 20 cP to about 200 cP with or without bioactive loading. In still another example, the insertable portion coating composition as applied to the insertable portion has a viscosity from about 30 cP to about 300 cP with or without bioactive loading.

As discussed previously herein, in examples, the dynamic contact angles, i.e., the contact angles which occurs in the course of wetting (advancing angle) or de-wetting (receding angle) of a surface for the biointerface/bioactive releasing membrane 70 has an advancing contact angle of about 1050 to about 130°, or 110° to about 120°. In yet another example, the dynamic contact angles, i.e., the contact angles which occurs in the course of wetting (advancing angle) or de-wetting (receding angle) of a surface for the biointerface/bioactive releasing membrane has a receding contact angle of about 40° to about 80°. In another example, the dynamic contact angles, i.e., the contact angles which occurs in the course of wetting (advancing angle) or de-wetting (receding angle) of a surface for the biointerface/bioactive releasing membrane 70 has a receding contact angle of about 450 to about 75°. In yet another example, the dynamic contact angles, i.e., the contact angles which occurs in the course of wetting (advancing angle) or de-wetting (receding angle) of a surface for the biointerface/bioactive releasing membrane 70 has a receding contact angle of about 50° to about 70°. In some examples, dynamic contact angle measurements and surface roughness on the bioactive releasing membrane 70 after placement on the analyte sensor and after sterilization can be carried out using a Sigma 701 force tensiometer and performing one or more of advancing contact angle measurements, receding contact angle measurements, hysteresis measurements, and combinations thereof.

In addition, in examples, the resulting bioactive releasing membrane 70 has a thickness of from about 20 μm to about 40 μm, including all ranges and subranges therebetween. In certain examples, the thickness of the insertable portion coating of the bioactive releasing membrane 70 can be from about 0.1, about 0.5, about 1, about 2, about 4, about 6, about 8 μm or less to about 10, about 15, about 20, about 30, about 40, about 50, about 75, about 100, about 125, about 150, about 175, about 200 or about 250 μm or more. In some of these examples, the thickness of the insertable portion coating can be sometimes from about 1 to about 5 μm, and sometimes from about 2 to about 7 μm. In other examples, the insertable portion coating can be from about 20 or about 25 μm to about 50, about 55, or about 60 μm thick.

In another example, the resulting insertable portion coating of the bioactive releasing membrane 70 has a length of about 1 mm to about 20 mm, including all ranges and subranges therebetween. In another example, the insertable portion coating has a length of about 2 mm to about 14 mm. In a further example, the insertable portion coating has a length of about 4 mm to about 12 mm. For example, the insertable portion coating has a length of at least about any of the following: 1, 2, 3, 4, 5, 6, 7, 8, 9, 10, 11, 12, 13, 14, 15, 16, 17, 18, and 19 mm and/or at most about 20, 19, 18, 17, 16, 15, 4, 13, 12, 11, 10, 9, 8, 7, 6, 5, 4, 3, and 2 mm (e.g., about 1-15 mm, about 5-18 mm, etc.).

The bioactive releasing membrane 70 is applied using at least one of spray coating, pad printing, fountain coating, bubble film coating, droplet coating, dip and wash, inverted dip and wash, or any combination thereof. For example, when dip coating combined with washing is used, a cross-section of the resulting insertable portion and insertable portion coating includes a plurality of concentric circles with defined edges or borders, as illustrated in FIGS. 2C, 31, and 3J. In contrast, when the resulting device is not dipped, the cross-section does not include distinct, defined edges or border but instead shows a gradual transition among the layers.

The bioactive releasing membrane 70 provides therapeutic benefits including reducing or delaying a subject's immune response in the tissue in which the insertable portion is inserted (e.g., the local tissue response).

Similarly, reducing or delaying the immune response improves sensitivity of the continuous analyte sensing devices discloses herein. For example, as shown in FIGS. 6B-6E, control sensors demonstrated significantly more sensitivity decline compared to bioactive-loaded sensors, and the sensor of the continuous analyte sensing devices described herein as including the insertable portion coating on the distal end of the insertable portion demonstrated the latest onset of sensitivity decline. Indeed, in the test results illustrated in FIG. 6D, at 15 days post-insertion, the control sensor had a sensitivity survival rate of 53%, the bioactive-loaded sensor had a sensitivity survival rate of 78%, and the bioactive-loaded sensor that only included the insertable portion coating on the distal end of the insertable portion had a sensitivity survival rate of 94%.

Methods for reducing or delaying the immune response include (i) providing the continuous analyte sensing device 100 described above, which is configured to deploy the insertable portion 102; (ii) causing formation (e.g., development or creation) of a tissue insertion volume in a subject at deployment of the insertable portion 102; (iii) releasing the at least one bioactive agent 110 from the bioactive releasing membrane 70 into the tissue insertion volume; and, as a result, (iv) reducing or delaying, in response to (iii) releasing the at least one bioactive agent, an immune response about the tissue insertion volume. As discussed previously herein, the tissue insertion volume includes subcutaneous or intradermal adipose or muscle tissue, and the composition of the tissue insertion volume varies based on the insertion site. In examples, the tissue insertion volume is greater than or equal to the insertable volume. In a further example, the at least one bioactive agent 110 is released from the bioactive releasing membrane 70 at an average release rate from about 0.1 μg to about 5 μg per day, including all ranges and subranges therebetween.

As a result of the methods described herein, in examples, the immune response is reduced or delayed for at least 7 days. In another example, the immune response is reduced or delayed for at least 10 days. In a further example, the immune response is reduced or delayed for at least 14 days. In yet another example, the immune response is reduced or delayed for at least 21 days. As such, the methods described herein reduce the immune response for at least 7, 8, 9, 10, 11, 12, 13, 14, 15, or 21 days, if not longer. As a result, the continuous analyte sensing devices disclosed herein are capable of bioactive release for at least 15 days post-insertion.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the disclosure disclosed herein. Consequently, it is not intended that this disclosure be limited to the specific examples disclosed herein, but that it cover all modifications and alternatives coming within the true scope and spirit of the disclosure.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures, but may also encompass combinations of elements of the various illustrated examples and aspects thereof.

1-126. (canceled)

127. A device for measurement of a concentration an analyte, the device comprising:
a sensor substrate comprising a distal end separated from a proximal end, and at least one sensor portion positioned between the distal end and the proximal end, the sensor portion configured to generate a signal associated with the concentration of the analyte; and
a bioactive releasing membrane adjacent the sensor substrate, the bioactive releasing membrane comprising at least one first bioactive agent capable of modifying a tissue response of a subject; the bioactive releasing membrane directly adjacent: a resistance membrane; an electrode membrane, or an interference membrane, the bioactive releasing membrane comprises at least one polymer segment, wherein the at least one polymer segment is selected from the group consisting of epoxides, polyolefins, polysiloxanes, polyamides, polystyrenes, polyacrylates, polyethers, polypyridines, polyesters, polyalkylesters, polyalkylcarbonates, polycarbonates, polyethylene vinyl acetate, polyvinyl alcohol, repeating zwitterionic groups, and copolymers thereof.

128. The device of claim 127, wherein the bioactive releasing membrane is positioned only at the distal end.

129. The device of claim 127, wherein the bioactive releasing membrane comprises at least one polymer segment, wherein the at least one polymer segment selected from the group consisting of epoxides, polyolefins, polysiloxanes, polyamides, polystyrenes, polyacrylates, polyethers, polyurethanes, polyurethane ureas, polypyridines, polyesters, polyalkylesters, polyalkylcarbonates, polycarbonates, polyethylene vinyl acetate, polyvinyl alcohol, and copolymers thereof.

130. The device of claim 127, wherein the bioactive releasing membrane comprises a soft segment and a hard segment comprising urethane groups, urea groups, or a combination of urethane groups and urea groups.

131. The device of claim 130, wherein the bioactive releasing membrane comprises a multicomponent soft segment comprising two or more different polymer segments.

132. The device of claim 130, wherein the multicomponent soft segment comprises a hydrophobic block and a hydrophilic block of a combination of at least one of a polysiloxane, a polyalkylcarbonate, and a polycarbonate with a polyalkylether, a polyalkylester.

133. The device of claim 130, wherein the soft segment comprises a combination of one or more of polysiloxane, polyalkylether, polyalkylester, polyalkylcarbonate, polycarbonate, and polysiloxane-polyalkylether segmented blocks and wherein the hard segment comprises at least one of norbornane diisocyanate (NBDI), isophorone diisocynate (IPDI), tolylene diisocynate (TDI), 1,3-phenylene diisocyanate (MPDI), trans-1,3-bis(isocynatomethyl) cyclohexane (1,3-H6XDI), bicyclohexylmethane-4,4′-diisocynate(HMDI), 4,4′-Diphenylmethane diisocynate (MDI), trans-1,4-bis(isocynatomethyl) cyclohexane (1,4-H6XDI), 1,4-cyclohexyl diisocynate (CHDI), 1,4-phenylene diisocynate (PPDI), 3,3′-Dimethyl-4,4′-biphenyldiisocyanate (TODI), and 1,6-hexamethylene diisocyanate (HDI).

134. The device of claim 127, wherein the weight/weight ratio of the at least one first bioactive agent to the bioactive releasing membrane is from about 0.1 to about 2.

135. The device of claim 127, wherein the at least one first bioactive agent comprises dexamethasone, a dexamethasone salt, a dexamethasone derivative, dexamethasone acetate, or a combination of a dexamethasone salt, a dexamethasone derivative, or a combination of dexamethasone acetate and dexamethasone.

136. The device of claim 127, further comprising a dissolvable coating adjacent the bioactive releasing membrane comprising a second releasable bioactive agent, wherein the first releasable bioactive agent is the same or different from the second releasable bioactive agent.

137. The device of claim 127, further comprising a diffusion adjustment membrane adjacent the bioactive releasing membrane, wherein diffusion adjustment membrane is different from the bioactive releasing membrane.

138. The device of claim 127, further comprising an electrically insulating end-cap adjacent the distal end.

139. The device of claim 138, wherein the electrically insulating end-cap is non-permeable to electrochemically active species or to the analyte.

140. The device of claim 138, wherein the electrically insulating end-cap extends longitudinally or circumferentially from the distal end.

141. The device of claim 138, wherein the electrically insulating end-cap extends from the distal end up to the sensor portion.

142. A device for measurement of a concentration of an analyte, the device comprising:
a sensor substrate comprising a distal end separated from a proximal end, and at least one sensor portion positioned between the distal end and the proximal end, the sensor portion configured to generate a signal associated with the concentration of the analyte; and
an electrically insulating end-cap adjacent the distal end and distal from the sensor portion;
a bioactive releasing membrane encapsulating the electrically insulating end-cap and distal from the sensor portion, the bioactive releasing membrane comprising at least one bioactive agent, the at least one bioactive agent configured to be released from the bioactive releasing membrane to modify a tissue response of a subject.

143. The device of claim 142, wherein the at least one bioactive agent comprises dexamethasone, a dexamethasone salt, a dexamethasone derivative, dexamethasone acetate, or a combination of a dexamethasone salt, a dexamethasone derivative or dexamethasone acetate with dexamethasone.

144. The device of claim 142, wherein the bioactive releasing membrane comprises at least one polymer segment, wherein the at least one polymer segment selected from the group consisting of epoxides, polyolefins, polyurethanes, polyurethaneureas, polysiloxanes, polyamides, polystyrenes, polyacrylates, polyethers, polypyridines, polyesters, polyalkylesters, polyalkylcarbonates, polycarbonates, polyethylene vinyl acetate, polyvinyl alcohol, repeating zwitterionic groups, and copolymers thereof.

145. The device of claim 142, wherein the electrically insulating end-cap extends longitudinally or circumferentially from the distal end.

146. The device of claim 142, wherein the end-cap is a thermoplastic silicone polycarbonate polyurethane.

147. The device of claim 142, wherein the weight/weight ratio of the at least one bioactive agent to the bioactive releasing membrane is from about 0.1 to about 2.
